(19) United States
(12) Patent Application Publication
(10) Pub. No.: US 2025/0382615 A1
(43) Pub. Date: Dec. 18, 2025

(54) NOVEL RNA THERAPEUTICS AND USES THEREOF

(71) Applicant: ELI LILLY AND COMPANY, Indianapolis, IN (US)

(72) Inventors: Marijean EGGEN, Brownsburg, IN (US); Patrick Joseph ANTONELLIS, Norwell, MA (US); Seth Andrew BAWEL, Brownsburg, IN (US); Thomas Patrick BEYER, Zionsville, IN (US); Christine Chih-Tao CHENG, Carmel, IN (US); Gregory Lawrence LACKNER, Indianapolis, IN (US); Rebecca Ruth MILES, Pendelton, IN (US); Jibo WANG, Carmel, IN (US); Takako WILSON, San Diego, CA (US)

(21) Appl. No.: 19/141,610

(22) PCT Filed: Dec. 22, 2023
(86) PCT No.: PCT/US2023/085647

§ 371 Date: Jun. 20, 2025

Related U.S. Application Data
(60) Provisional application No. 63/435,069, filed on Dec. 23, 2022.

(51) Int. Cl. C12N 15/113; A61P 9/06

(52) U.S. Cl. CPC C12N 15/113; A61P 9/06; C12N 2310/11; C12N 2310/14; C12N 2310/315; C12N 2310/321; C12N 2310/322; C12N 2310/351; C12N 2320/32

(57) ABSTRACT

The present invention relates to novel therapeutic compounds, known as RNAi agents, that decrease expression of the ANGPTL8 receptor (expressed by the ANGPTL8 gene), thereby decreasing expression of mRNA and protein expression. Such RNAi agents are useful in the treatment of diseases involving the regulation of ANGPTL8 expression and function, such as dyslipidemia, a cardiovascular disorder, or a cardiometabolic disorder.

### BACKGROUND

The present disclosure relates to novel RNAi agents designed to decrease the expression of ANGPTL8 in the liver, where the RNAi agents comprise delivery moieties conjugated to oligonucleotides optionally via a linker. The RNAi agents are useful in the treatment of diseases involving the regulation of ANGPTL8 expression.

Angiopoietin-like protein 8 (ANGPTL8) is mainly expressed in liver and adipose tissue and it plays an important role in triglyceride metabolism. ANGPTL8, together with ANGPTL3 or ANGPTL4, is thought to regulate triglyceride levels by inhibiting the enzymatic activity of lipoprotein lipase (LPL), which, when active, hydrolyzes triglycerides 10 and decreases circulating plasma triglycerides. Increased levels of ANGPTL8 are observed or associated with cardiovascular disease, diabetes, dyslipidemia (including high triglyceride levels), aberrant renal function, hypertension, nonalcoholic fatty liver disease such as nonalcoholic steatohepatitis (NASH), and obesity.

ANGPTL8 siRNAs and ASOs have been described, such as those disclosed in WO2020/104649 A2, but none have progressed for treatment in patients. Using the ANGPTL8 RNAi agents herein to decrease expression of ANGPTL8 can be employed, e.g., to treat cardiometabolic and related disorders such as dyslipidemia, in patients in need thereof.

### SUMMARY OF INVENTION

In one aspect, provided herein are RNAi agents for reducing ANGPTL8 gene expression, wherein the RNAi agent comprises a delivery moiety of Formula I conjugated to R, wherein R is a double stranded RNA (dsRNA) comprising an antisense strand and a sense strand:

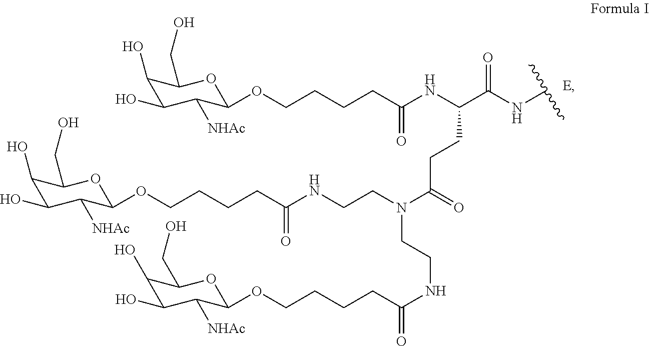

wherein R is conjugated to connection point E of Formula I, optionally via a linker, wherein the sense strand and the antisense strand form a duplex region, and wherein the antisense strand comprises a region of complementarity to an ANGPTL8 mRNA target sequence of SEQ ID NO: 511, and wherein the sense and antisense strand each optionally comprise one or more modified nucleotides and optionally one or more modified internucleotide linkages. In some embodiments, Formula I is conjugated to the sense strand, optionally via a linker. In some embodiments, Formula I is conjugated to the 3′ terminal nucleotide of the sense strand, optionally via a linker.

In an embodiment, provided herein are RNAi agents for reducing ANGPTL8 gene expression, wherein the RNAi agent comprises a delivery moiety of Formula I conjugated to R, wherein R is a double stranded RNA (dsRNA) comprising an antisense strand and a sense strand:

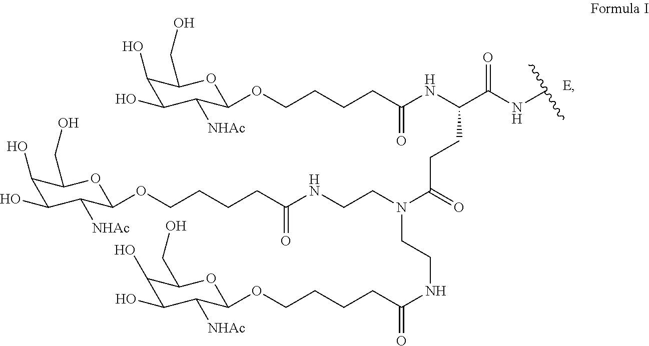

wherein R is conjugated to connection point E of Formula I, optionally via a linker, wherein the sense strand and the antisense strand form a duplex region, and wherein the antisense strand comprises any one of SEQ ID NOs: 6, 7, 8, 9, 10, 11, 12, 13, 14, or 15, or a sequence having 90% sequence identity thereto, or an antisense strand sequence as set forth in Tables 3A, 3B, and 4 or a sequence having 90% sequence identity thereto, and wherein the sense and antisense strand each optionally comprise one or more modified nucleotides and optionally one or more modified internucleotide linkages. In some embodiments, Formula I is conjugated to the sense strand, optionally via a linker. In some embodiments, Formula I is conjugated to the 3′ terminal nucleotide of the sense strand, optionally via a linker.

In some embodiments, the antisense strand is 15 to 50 nucleotides in length. In some embodiments, the sense strand is 15 to 50 nucleotides in length. In some embodiments, the antisense strand is between 18 and 23 nucleotides in length. In some embodiments, the sense strand is between 18 and 21 nucleotides in length. In some embodiments, the antisense strand is 23 nucleotides in length and the sense strand is 21 nucleotides in length.

In some embodiments, the sense strand or the antisense strand comprises a sequence selected from Table 2A, Table 2B, Table 3A, Table 3B, or Table 4 disclosed herein. In some embodiments, the sense strand and the antisense strand comprise a sequence selected from Table 2A, Table 2B, Table 3A, Table 3B, or Table 4 disclosed herein.

In some embodiments, R is conjugated to Formula I via a linker. In further embodiments, the linker comprises a linker of Formula II having connection points A and B or the linker comprises Formula III having connection points C and D, and wherein:

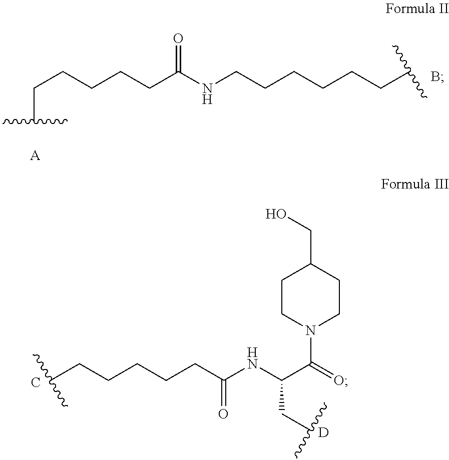

a. Formula I, at connection point E, conjugated to Formula II at connection point A and Formula II is conjugated to a phosphate group at connection point B, and the phosphate group is further conjugated to R; or
b. Formula I, at connection point E, conjugated to Formula III at connection point C and Formula III is conjugated to a phosphate group at connection point D, and the phosphate group is further conjugated to R.

In another aspect, provided herein are pharmaceutical composition comprising the ANGPTL8 RNAi agent described herein and one or more pharmaceutically acceptable excipients.

In another aspect, provided herein are methods of treating cardiovascular disease, diabetes, dyslipidemia (including high triglyceride levels), aberrant renal function, hypertension, nonalcoholic fatty liver disease such as nonalcoholic steatohepatitis (NASH), or obesity in a patient in need thereof, comprising administering to the patient a ANGPTL8 RNAi agent or pharmaceutical composition thereof described herein.

In another aspect, provided herein are ANGPTL8 RNAi agent for use in a therapy. Also provided herein are uses of ANGPTL8 RNAi agent in the manufacture of a medicament for the treatment of cardiovascular disease, diabetes, dyslipidemia (including high triglyceride levels), aberrant renal function, hypertension, nonalcoholic fatty liver disease such as nonalcoholic steatohepatitis (NASH), or obesity.

### DETAILED DESCRIPTION

Such siRNAs may exhibit one or more of, e.g., as compared to other liver targeted siRNAs such as ANGPTL8 siRNAs comprising a different delivery ligand, a different sequence, a differently modified sequence, or as compared to treatment with a vehicle control: improved knockdown in the liver; improved tissue exposure, improved exposure in liver hepatocytes; an improved durable response; an improved pharmacokinetic profile; fewer off target effects, and/or an improved toxicity profile. Other embodiments of the ANGPTL8 RNAi agents herein may include one or more of fewer side effects as compared to statins or other standard of care; an improved toxicity profile; an improved safety profile; improved tolerability or compliance; and/or improved liver function tests. Still other siRNAs herein may have other benefits, e.g., in combination with any of the preceding or as a stand-alone benefit, including improved and/or simplified synthesis, synthetic processes with fewer degradation products; or any combination thereof.

The RNAi agents herein comprise a sense strand and an antisense strand, wherein each is an oligonucleotide. In some embodiments, the RNAi agent described herein also comprises a delivery moiety. As used herein, “nucleotide” means an organic compound having a nucleoside (a nucleobase such as, for example, adenine, cytosine, guanine, thymine, or uracil; and a pentose sugar such as, for example, ribose or 2′-deoxyribose) and a phosphate group. A “nucleotide” can serve as a monomeric unit of nucleic acid polymers such as deoxyribonucleic acid (DNA) and ribonucleic acid (RNA).

As used herein, “oligonucleotide” means a short nucleic acid compound (e.g., less than about 100 nucleotides in length). An oligonucleotide may be single-stranded (ss) or double stranded (ds). An oligonucleotide may or may not have duplex regions. As a set of non-limiting examples, an oligonucleotide may be, but is not limited to, a small interfering RNA (siRNA), microRNA (miRNA), short hairpin RNA (shRNA), Dicer substrate interfering RNA (DsiRNA), or antisense oligonucleotide (ASO).

As used herein, “ribonucleotide” means a nucleotide having a ribose as its pentose sugar, which contains a hydroxyl group at its 2′ position. A modified ribonucleotide is a ribonucleotide having one or more modifications or substitutions of atoms other than hydrogen at the 2′ position, including modifications or substitutions in or of the nucleobase, sugar, or phosphate group.

As used herein, “modified internucleotide linkage” means an internucleotide linkage having one or more chemical modifications when compared with a reference internucleotide linkage having a phosphodiester bond. A modified internucleotide linkage can be a non-naturally occurring linkage.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide. A modified nucleotide can be a non-naturally occurring nucleotide. A modified nucleotide can have, for example, one or more chemical modification in its sugar, nucleobase, and/or phosphate group. Additionally, or alternatively, a modified nucleotide can have one or more chemical moieties conjugated to a corresponding reference nucleotide.

The term “percentage sequence identity” with respect to a reference nucleic acid sequence is defined as the percentage of nucleotides, nucleosides, or nucleobases in a candidate sequence that are identical with the nucleotides, nucleosides, or nucleobases in the reference nucleic acid sequence, after optimally aligning the sequences and introducing gaps or overhangs, if necessary, to achieve the maximum percent sequence identity. Alignment for purposes of determining percent nucleic acid sequence identity can be achieved in various ways that are within the skill in the art, for instance, using publicly available computer software programs, for example, those described in Current Protocols in Molecular Biology (Ausubel et al., eds., 1987, Supp. 30, section 7.7.18, Table 7.7.1), and including BLAST, BLAST-2, ALIGN, Clustal W2.0 or Clustal X2.0 or Megalign (DNASTAR) software. In one embodiment herein, sequence identity is calculated use Clustal W2.0 or Clustal X2.0. In another embodiment, sequence identity is calculated using Clustal W2.0. In another embodiment, sequence identity is calculated using Clustal X2.0. Those skilled in the art can determine appropriate parameters for measuring alignment, including any algorithms needed to achieve maximal alignment over the full length of the sequences being compared. Percentage of “sequence identity” can be determined by comparing two optimally aligned sequences over a comparison window, where the fragment of the nucleic acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical nucleotide, nucleoside, or nucleobase occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence. In some embodiments, percent sequence identity is the percent of nucleotide residues that are identical between two strands using the PID3 calculation, which is the number of identical nucleotide residues divided by the total number of nucleotides of the shortest of the two sequences, multiplied by 100. See, e.g., Raghava, G., Barton, G. J. Quantification of the variation in percentage identity for protein sequence alignments. BMC Bioinformatics 7, 415 (2006).

As used herein, “phosphate analog” means a chemical moiety that mimics the electrostatic and/or steric properties of a phosphate group. In some embodiments, a phosphate analog is positioned at the 5′ terminal nucleotide of an oligonucleotide in place of a 5′-phosphate. A 5′ phosphate analog can include a phosphatase-resistant linkage. Examples of phosphate analogs include, but are not limited to, 5′ phosphonates, such as 5′ methylene phosphonate (5′-MP) and 5′-(E)-vinylphosphonate (5′-VP). An oligonucleotide can have a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e g., Intl. Patent Application Publication No. WO 2018/045317. Other modifications have been developed for the 5′ end of oligonucleotides (see, e.g., Intl. Patent Application No. WO 2011/133871; U.S. Pat. No. 8,927,513; and Prakash et al. (2015) Nuc. Acids Res. 43:2993-3011).

As used herein, “region of complementarity” means a nucleotide sequence of a nucleic acid (e.g., a double stranded oligonucleotide) that is sufficiently complementary to an antiparallel nucleotide sequence to permit hybridization between the two sequences of nucleotides under appropriate hybridization conditions (e.g., in a phosphate buffer, in a cell, etc.). In some embodiments, an oligonucleotide herein includes a targeting sequence having a region of complementary to a mRNA target sequence.

As used herein, “duplex,” in reference to nucleic acids or oligonucleotides, such as a sense strand or an antisense strand means a structure formed through hydrogen bonds of complementary base pairing of two antiparallel sequences of nucleotides under suitable conditions to promote such a structure. A duplex may form despite not having full complementarity between the two strands, or when an abasic nucleotide is present. A Duplex No:, as shown herein, e.g., in Table 2A, Table 2B, Table 3A Table 3B, or Table 4 corresponds to a specific sense and antisense strand that comprise a given RNAi agent.

RNA interference is a specialized cellular process that utilizes RISC for degrading RNA in a sequence dependent manner. As used herein, “RNAi agent” comprises either (a) a double stranded oligonucleotide having a sense strand (passenger) and antisense strand (guide), in which the antisense strand or part of the antisense strand is used by the Argonaute 2 (Ago2) endonuclease in the cleavage of a target mRNA or (b) a single stranded oligonucleotide having a single antisense strand, where that antisense strand (or part of that antisense strand) is used by the Ago2 endonuclease in the cleavage of a target mRNA. In some embodiments, RNAi agent comprises a delivery moiety.

As used herein, a bond illustrated as [symbol] indicates a connection point as described therein. For example, if a generic variable, e.g., X, is stated to be attached at the connection point E as shown below, this is intended to show X is bonded to the atom of the connection point (see the scheme below).

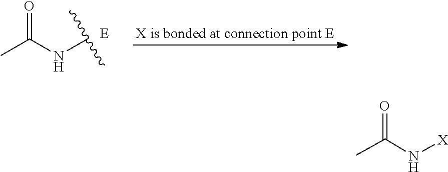

As used herein, “treatment” or “treating” refers to all processes wherein there may be a slowing, controlling, delaying, or stopping of the progression of the disorders or disease disclosed herein, or ameliorating disorder or disease symptoms, and need not indicate a total elimination of all disorder or disease symptoms. Treatment includes administration of an RNAi agent or pharmaceutical composition thereof for treatment of a disease or condition in a mammal including a human.

An “effective amount” refers to an amount necessary (for periods of time and for the means of administration) to achieve the desired therapeutic result. An effective amount of a RNAi agent may vary according to factors such as the disease state, age, sex, and weight of the individual, and the ability of the RNAi agent to elicit a desired response in the individual. An effective amount is also one in which any toxic or detrimental effects of the RNAi agent are outweighed by the therapeutically beneficial effects.

Provided herein are RNAi agents for reducing ANGPTL8 gene expression, wherein the RNAi agent comprises a delivery moiety of Formula I conjugated to R, wherein R is a double stranded RNA (dsRNA) comprising an antisense strand and a sense strand:

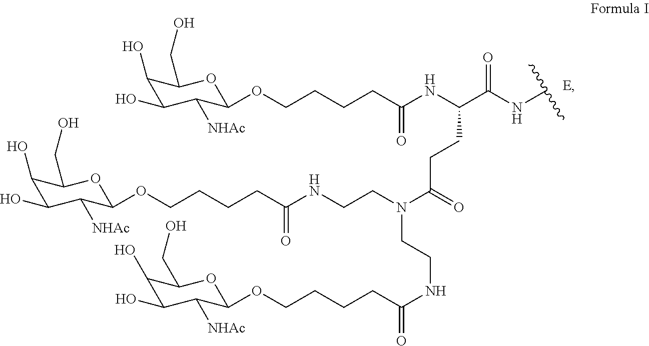

wherein R is conjugated to connection point E of Formula I, optionally via a linker, wherein the sense strand and the antisense strand form a duplex region, and wherein the antisense strand comprises a region of complementarity to a ANGPTL8 mRNA target sequence of SEQ ID NO: 511, and wherein the sense and antisense strand each optionally comprise one or more modified nucleotides and one or more modified internucleotide linkages.

Also provided here are RNAi agents for reducing ANGPTL8 gene expression, wherein the RNAi agent comprises a delivery moiety of Formula Ia conjugated to R, wherein R comprises an antisense strand and a sense strand:

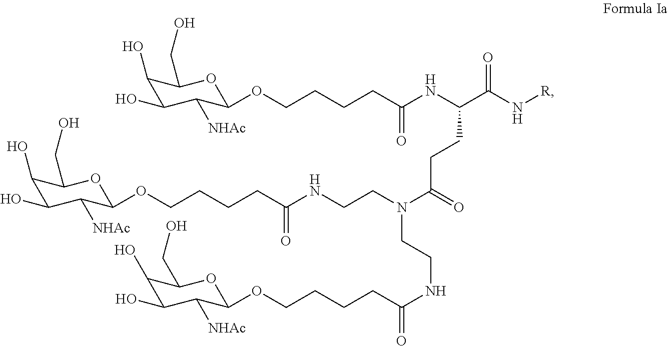

wherein R is optionally conjugated to Formula Ia via a linker, wherein the sense strand and the antisense strand form a duplex region, and wherein the antisense strand comprises a region of complementarity to a ANGPTL8 mRNA target sequence of SEQ ID NO: 511, and wherein the sense and antisense strand each optionally comprise one or more modified nucleotides and one or more modified internucleotide linkages. Disclosed herein are RNAi agents for reducing ANGPTL8 gene expression, wherein the RNAi agents comprise a sense strand and an antisense strand, wherein the sense strand and the antisense strand form a duplex region, and wherein the antisense strand comprises at least 15 nucleotides as set forth in an antisense strand sequence disclosed herein, and wherein the sense strand and/or the antisense strand each optionally comprise one or more modified nucleotides and/or modified internucleotide linkages. In further embodiments, the antisense strand comprises at least 15 nucleotides of an antisense strand sequence in Table 2A, Table 2B, Table 3A, Table 3B, or Table 4. In further embodiments, the RNAi agent reduces ANGPTL8 gene expression by about 50% or greater in a cell expressing ANGPTL8, as compared to a control. In further embodiments, the RNAi agent reduces ANGPTL8 gene expression by reducing the level of ANGPTL8 mRNA transcript, the level of ANGPTL8 protein, or both.

In further embodiments, the antisense strand is 15 to 25 nucleotides in length, and/or the sense strand is 15 to 25 nucleotides in length. In further embodiments, the antisense strand is between 18 and 23 nucleotides in length. In further embodiments, the sense strand is between 18 and 21 nucleotides in length.

In further embodiments, the RNAi agent comprises at least 18 contiguous nucleotides of an antisense strand sequence set forth in Table 2A, Table 2B, Table 3A, Table 3B, or Table 4.

In further embodiments, the antisense strand of the RNAi agent is 23 nucleotides in length. In still further embodiments, the sense strand is 21 nucleotides in length. In another embodiment, the sense and antisense strand comprise a sequence selected from the sequences set forth in Table 2A, Table 2B, Table 3A, Table 3B, or Table 4.

The sense strand and the antisense strand of the RNAi agents disclosed herein do not require full complementarity. Accordingly, in the RNAi agents disclosed herein, the duplex region between the sense strand and the antisense strand comprises 0, 1, 2, or 3 mismatches between the sense strand and the antisense strand. In further embodiments, the duplex region between the sense strand and the antisense strand consists of 0, 1, 2, or 3 mismatches between the sense strand and the antisense strand.

In further embodiments, the sense strand and the antisense strand each independently comprise one or more modified nucleotides, such as 2′ fluoro modified nucleotides or 2′-O-methyl modified nucleotides. In still further embodiments of the RNAi agents disclosed herein, each nucleotide of the sense strand and each nucleotide of the antisense strand is a modified nucleotide. In further embodiments, each nucleotide is a 2′ fluoro modified nucleotide or a 2′-O-methyl modified nucleotide.

In further embodiments, the antisense strand has a sequence as set forth in an antisense strand sequence in Table 2A, Table 2B, or Table 4, or a sequence having at least 90% sequence identity thereto, or an antisense strand sequence in Table 3A or Table 3B, or a sequence having at least 90% sequence identity thereto. In other embodiments, the antisense strand sequence or the sense strand sequence in Table 2A or Table 2B or Table 3A or Table 3B or Table 4 is independently a sequence that is at least 80, 81, 82, 83, 84, 85, 86, 87, 88, 89, 90, 91, 92, 93, 94, 95, 96, 97, 98, or 99 percent identical thereto.

In further embodiments of the RNAi agents disclosed herein, the antisense strand is 23 nucleotides in length, each nucleotide of the antisense strand is a modified nucleotide, and the 2′ fluoro modified nucleotides may appear at different positions than is shown in the sequences in Table 2A, Table 2B, Table 3A, Table 3B, or Table 4. In an embodiment, the 2′ Fluoro modified nucleotides are present at
a. Positions 2, 3, 7, 14, and 16 from the 5′ end of the antisense strand; or
b. Positions 2, 5, 7, 14, and 16 from the 5′ end of the antisense strand; or
c. Positions 2, 3, 8, 14, and 16 from the 5′ end of the antisense strand; or
d. Positions 2, 5, 8, 14, and 16 from the 5′ end of the antisense strand; or
e. Positions 2, 6, 14, and 16 from the 5′ end of the antisense strand.
   In further embodiments, the nucleotides that are not 2′ fluoro modified nucleotides are 2′-O-methyl modified nucleotides.

In further embodiments of the RNAi agents disclosed herein, the sense strand and antisense strand each independently comprise one or more modified internucleotide linkages, and each modified internucleotide linkage is a phosphorothioate linkage. In further embodiments, the sense strand and antisense strand each independently comprise four phosphorothioate linkages. In still further embodiments, the two terminal nucleotides at each of the 5′ and 3′ ends of each of the sense and antisense strand are phosphorothioate linkages.

In other embodiments, the 5′ nucleotide of the antisense strand comprises a naturally occurring OH group, or is modified to contain a phosphate group or a phosphate analog. As used herein, “phosphate analog” means a chemical moiety that mimics the electrostatic and/or steric properties of a phosphate group. In some embodiments, a phosphate analog is positioned at the 5′ terminal nucleotide of an oligonucleotide in place of a 5′-phosphate. A 5′ phosphate analog can include a phosphatase-resistant linkage. Examples of phosphate analogs include, but are not limited to, 5′ phosphonates, such as 5′ methylene phosphonate (5′-MP) and 5′-(E)-vinylphosphonate (5′-VP). An oligonucleotide can have a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e g., Intl. Patent Application Publication No. WO 2018/045317. Other modifications have been developed for the 5′ end of oligonucleotides (see, e.g., Intl. Patent Application No. WO 2011/133871; U.S. Pat. No. 8,927,513; and Prakash et al. (2015) Nuc. Acids Res. 43:2993-3011).

In further embodiments, the 5′ terminal nucleotide of the antisense strand may comprise a further modification, wherein the 5′ terminal nucleotide contains as a 5′ a vinyl phosphonate, a phosphate, or a hydroxyl group. In other embodiments, the phosphate group listed at the 5′ end of the recited SEQ ID NO: is removed and replaced with an OH. In other embodiments, the phosphate group listed at the 5′ end of the recited SEQ ID NO: is replaced with a 5′ vinylphosphonate.

In further embodiments, 1, 2, or 3 mismatches are introduced into the sense strand sequence of Table 2A Table 2B, Table 3A, Table 3B. In further embodiments, 1, 2, or both terminal nucleotides of 5′ end of the antisense strand are changed.

In some embodiments of the RNAi agents herein, the antisense strand comprises a first nucleic acid sequence that has at least 90% sequence identity to an antisense sequence corresponding to a Duplex NO: in Table 2A, Table 2B, Table 3A, Table 3B, or Table 4 and the sense strand comprises a second nucleic acid sequence that has at least 90% sequence identity to a sense sequence corresponding to the same Duplex No: in Table 2A, Table 2B, Table 3A, Table 3B, or Table 4. For example, in one embodiment, the antisense strand comprises a first nucleic acid sequence that has at least 90% sequence identity to an antisense sequence corresponding to a Duplex NO: 1 in Table 2A, that is, a first nucleic acid sequence that has at least 90% sequence identity to SEQ ID NO:6, and the sense strand comprises a second nucleic acid sequence that has at least 90% sequence identity to a sense sequence corresponding to Duplex No: 1 in Table 2A, that is, SEQ ID NO: 1. In further embodiments, the 5′ phosphate of the antisense strand is further modified/replaced, and is a 5′ vinylphosphonate or an OH group.

In further embodiments, the 5′ terminal nucleotide of the antisense strand is substituted such that the final sequence contains a vinylphosphonate, a phosphate group, or an OH group.

In other embodiments disclosed herein are RNAi agents having a delivery moiety of Formula I conjugated to R:

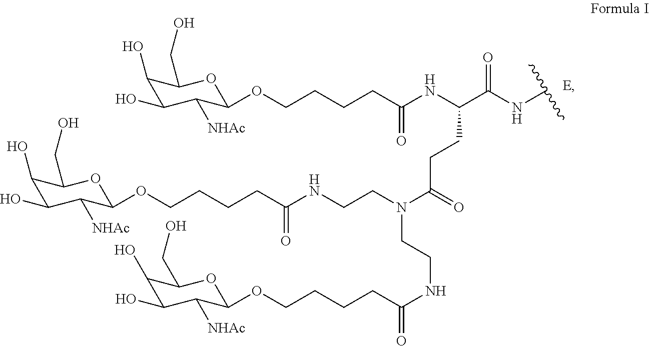

wherein R is a dsRNA comprises a sense strand and an antisense strand, wherein the antisense strand comprises at least 15 contiguous nucleotides that have complementarity to ANGPTL8 mRNA target sequence of SEQ ID NO: 511, and wherein the sense strand and the antisense strand form a region of complementarity of at least 15 nucleotides, and wherein the sense strand and antisense strand are each independently 18 to 23 nucleotides in length, and optionally wherein the sense strand and antisense strand each independently comprise one or more modified nucleotides, and optionally wherein the sense strand and the antisense strand each independently comprise one or more modified internucleotide linkages, and wherein R is optionally conjugated to Formula I via a linker. In further embodiments, the sense or the antisense strand is selected from a Table 2A, Table 2B, Table 3A, Table 3B, or Table 4 disclosed herein. In other embodiments, the antisense or antisense strand of the RNAi agent has an antisense strand sequence and/or a sense strand sequence of at least 80%, 81%, 82%, 83%, 84%, 85%, 86%, 87%, 88%, 89%, 90%, 91%, 92%, 93%, 94%, 95%, 96%, 97%, 98%, or 99% sequence identity to the corresponding sequence selected from a Table 2A, Table 2B, Table 3A, Table 3B, or Table 4 herein.

In other embodiments, the RNAi agent disclosed herein comprises a linker. In other further embodiments R is conjugated to Formula I via a linker. In further embodiments, the linker comprises a linker of Formula II having connection points A and B or the linker comprises Formula III having connection points C and D, and wherein:

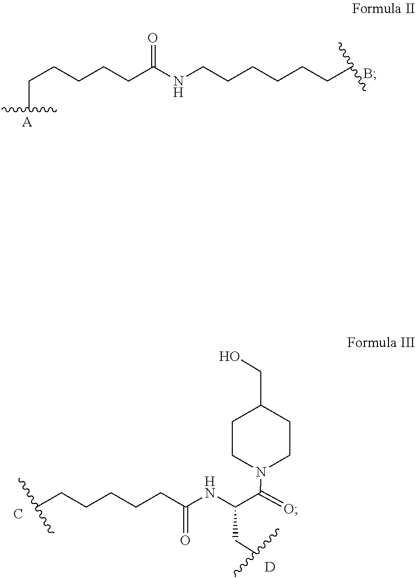

a. the RNAi agent comprises Formula I, at connection point E, conjugated to Formula II at connection point A and Formula II is conjugated to a phosphate group at connection point B, and the phosphate group is conjugated to R; or
b. the RNAi agent comprises Formula I, at connection point E, conjugated to Formula III at connection point C and Formula III is conjugated to a phosphate group at connection point D, and the phosphate group is further conjugated to R.

In other embodiments wherein the RNAi agent comprises a linker, R is conjugated to Formula I via a linker, and the linker is a linker comprising Formula III having connection points C and D:

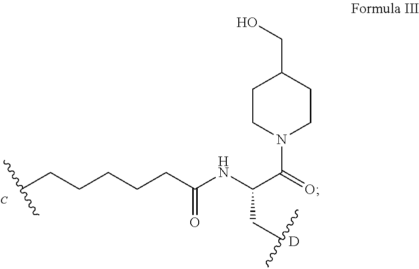

and wherein the RNAi agent comprises Formula I conjugated to Formula III at connection point C and Formula III is conjugated to a phosphate group at connection point D, and the phosphate group is further conjugated to R.

The sense strand and antisense strand of RNAi agent can be synthesized using any nucleic acid polymerization methods known in the art, for example, solid-phase synthesis by employing phosphoramidite chemistry methodology (e.g., Current Protocols in Nucleic Acid Chemistry, Beaucage, S. L. et al. (Edrs.), John Wiley & Sons, Inc., New York, NY, USA), H-phosphonate, phosphotriester chemistry, or enzymatic synthesis. Automated commercial synthesizers can be used, for example, MerMade™ 12 from LGC Biosearch Technologies, or other synthesizers from BioAutomation or Applied Biosystems. Phosphorothioate linkages can be introduced using a sulfurizing reagent such as phenylacetyl disulfide or DDTT (((dimethylaminomethylidene) amino)-3H-1,2,4-dithiazaoline-3-thione). It is well known to use similar techniques and commercially available modified amidites and controlled-pore glass (CPG) products to synthesize modified oligonucleotides.

In still other embodiments, the RNAi agent is capable of decreasing expression of the ANGPTL8 gene in a liver cell. In other embodiments, the RNAi agents disclosed herein are for use in therapy. In further embodiments, the use is for the treatment of dyslipidemia such as high plasma triglyceride levels. In other embodiments, the RNAi agents disclosed herein are for use in the treatment of cardiovascular disease. In other embodiments herein, the RNAi agents are for use in preventing a cardiovascular event. In further embodiments, the cardiovascular event is myocardial infarction. In other embodiments, the use is for decreasing hospitalizations related to cardiovascular disease or events. In other embodiments, the use is for treating non-alcoholic fatty liver disease (NAFLD). In further embodiments, wherein the NAFLD is non-alcoholic steatohepatitis (NASH). In other embodiments, the use is for decreasing inhibition of lipoprotein lipase (LPL). In further embodiments, the use is for increasing catabolism of triglyceride rich lipoproteins. In other embodiments, the RNAi agents are for use in treating a liver disease in a patient that would benefit from decreasing expression levels of ANGPTL8. In other embodiments, the use is for treatment of any of the preceding, after statin use failed to control one or more symptoms, e.g. failed to reduce one or more of elevated total-C, LDL-C, apo B, and/or failed to increase HDLC. In other embodiments, the use is for the treatment of any of the preceding, in patients that are statin intolerant; in further embodiments, the use is for lowering LDL-C in patients that are statin intolerant. In further embodiments, the use is for any of the preceding uses, after alteration of diet failed to control one or more symptoms. In other further embodiments, the use is for any of the preceding uses, as an adjunct therapy to diet.

The RNAi agents may be formulated into pharmaceutical compositions. Accordingly, disclosed herein are pharmaceutical compositions comprising the RNAi agent disclosed herein, and one or more pharmaceutically acceptable excipients. Pharmaceutical compositions can be prepared by methods well known in the art (e.g., Remington: The Science and Practice of Pharmacy, 23rd edition (2020), A. Loyd et al., Academic Press).

In other embodiment are uses of the RNAi agents herein for the manufacture of a medicament for the treatment of dyslipidemia or any of the uses recited in the preceding paragraph.

In other embodiments are methods of treating dyslipidemia, in patients in need thereof, comprising administering an RNAi agent disclosed herein, or a pharmaceutical composition thereof, to the patient. In other embodiments are methods of treating dyslipidemia, or any of the uses recited above, in patients in need thereof, or a pharmaceutical composition thereof. In other embodiments are methods of treating a patient unable to achieve lipid levels following statin and/or diet therapy, comprising administering to the patient an RNAi agent disclosed herein, or a pharmaceutical composition thereof.

The RNAi agent can be administered to the patient intravenously or subcutaneously.

RNAi dosage regimens may be adjusted to provide the optimum desired response (e.g., a therapeutic response). For example, a single bolus may be administered, several divided doses may be administered over time, or the dose may be proportionally reduced or increased as indicated by the exigencies of the therapeutic situation.

Dosage values may vary with the type and severity of the condition to be alleviated. It is further understood that for any particular subject, specific dosage regimens should be adjusted over time according to the individual need and the professional judgment of the person administering or supervising the administration of the compositions.

In other embodiments are methods of decreasing ANGPTL8 expression in a cell, comprising contacting the cell with an RNAi agent disclosed herein, and incubating the cell for a time sufficient for decreasing the level of ANGPTL8 mRNA by at least 50% as compared to an untreated or control treated cell.

Certain abbreviations are defined as follows: “1,2-DCE” refers to 1,2-dichloroethane; “DCM” refers to dichloromethane; “DIEA” refers to N,N-diisopropylethylamine; “DMF” refers to N,N-dimethylformamide; “DMAP” refers to 4-dimethylaminopyridine; “DMTCl” refers to 4,4′-dimethoxytrityl chloride; “DPP4” refers to dipeptidyl peptidase; “EDC” refers to 1-ethyl-3-(3-dimethylaminopropyl) carbodiimide; “EtOAc” refers to ethyl acetate; “GalNAc” refers to N-acetylgalactosamine; “HATU” refers to 1-[bis(dimethylamino)methylene]-1H-1,2,3-triazolo[4,5-b]pyridinium 3-oxide hexafluorophosphate; “HBTU” refers to O-(benzotriazol-1-yl)-N,N,N′,N′-tetramethyluronium hexafluorophosphate; “HOBt” refers to 1-hydroxybenzotriazole hydrate; “HPRT” refers to hypoxanthine-guanine phosphoribosyltransferase; “IPA” refers to isopropanol and isopropyl alcohol; “LDHA” refers to lactate dehydrogenase-A; “MeCN” refers to acetonitrile; “MeOH” refers to methanol and methyl alcohol; “MWCO” refers to molecular weight cut-off; “NHS” refers to N-hydroxysuccinimide; “OD” refers to optical density; “PBS” refers to phosphate-buffered saline; “PhSiH₃” refers to phenylsilane; “PTS” refers to portable endotoxin testing system; “siRNA” refers to small interfering ribonucleic acid; “TEA” refers to triethylamine; “TFA” refers to trifluoroacetic acid; “THF” refers to tetrahydrofuran; “TLC” refers to thin line chromatography; and “TMP” refers to 2,2,6,6-tetramethylpiperidine.

A delivery moiety comprising Formula I may be made by the following nonlimiting synthetic steps and schemes.

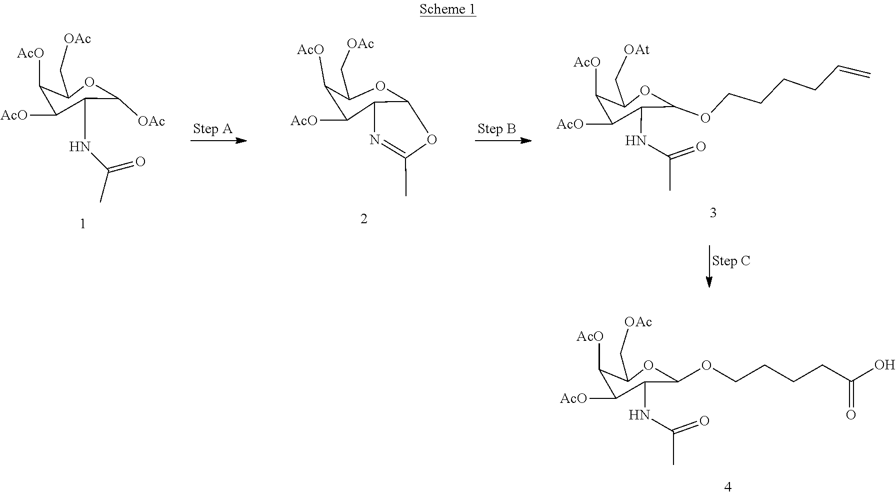

Scheme 1, step A, depicts the cyclization of compound (1) using trimethyl trifluoromethanesulfonate in a solvent such as 1,2-DCE to give compound (2). Step B shows the addition of hex-5-en-1-ol to compound (2) using trimethylsilyl trifluoromethanesulfonate in a solvent such as 1,2-DCE to give compound (3). The oxidation of compound (3) using an appropriate oxidizing agent such as sodium periodate with a catalyst such as ruthenium (III) chloride to give compound (4) is shown in step C.

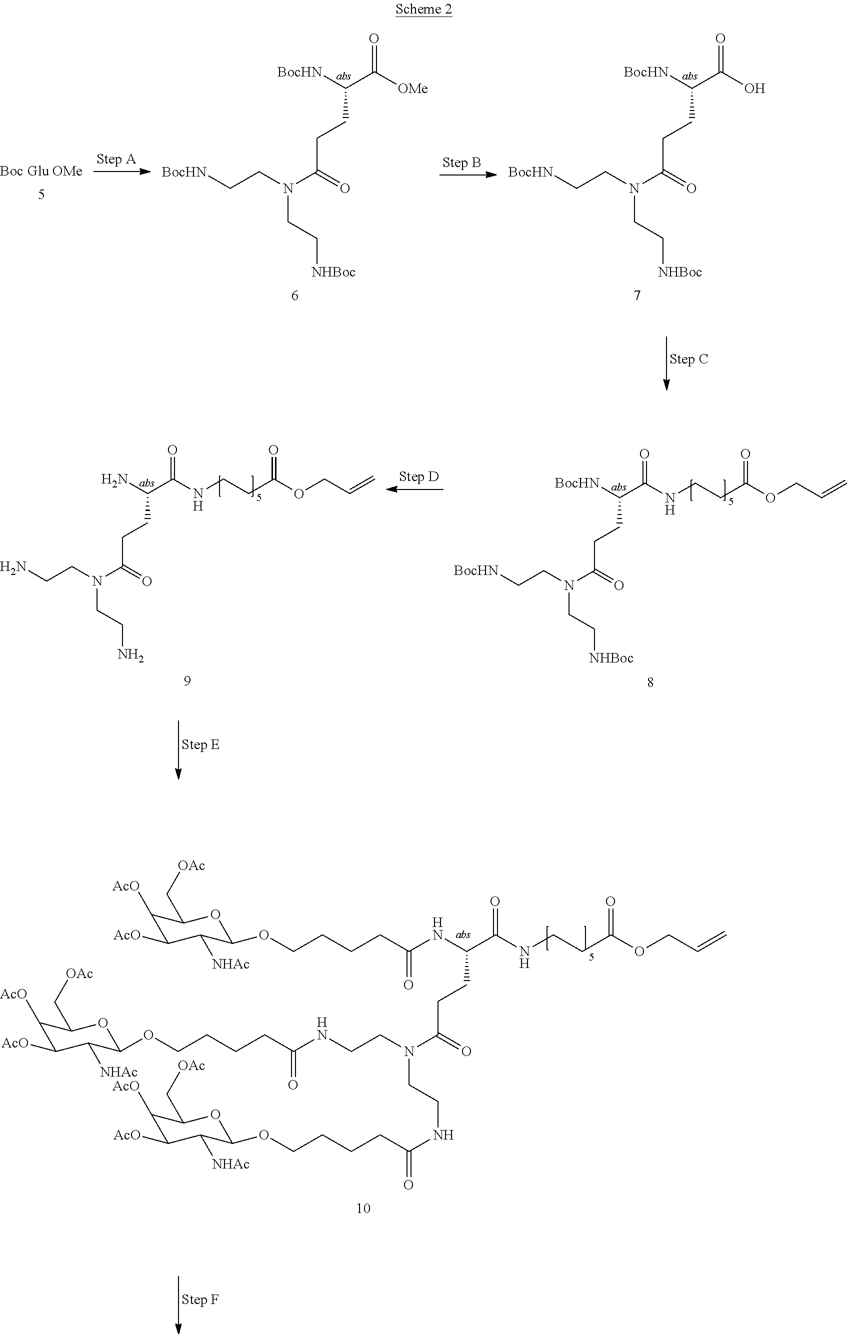

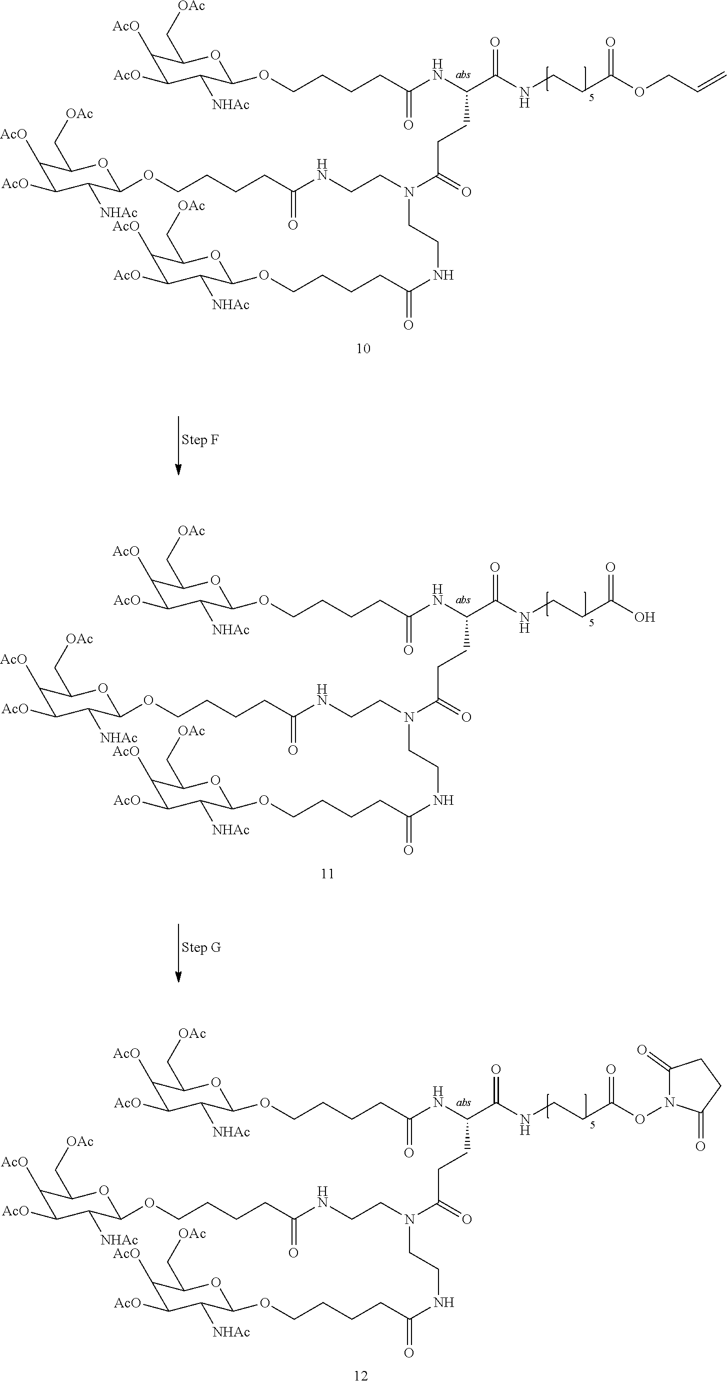

Scheme 2, step A, shows an amide coupling between compound (5) and tert-butyl N-[2-[2-(tert-butoxycarbonylamino)ethylamino]ethyl]carbamate using HBTU and HOBt with an appropriate base such as DIEA in a solvent such as DMF to give compound (6). Step B depicts a basic hydrolysis of compound (6) using a base such as aqueous NaOH in a THF and MeOH solvent system to give compound (7). Step C shows an amide coupling between compound (7) and allyl 11-aminoundecanoate hydrochloride using HATU with an appropriate base such as DIEA in a solvent such as DMF to give compound (8). Step D shows the acidic deprotection of compound (8) with TFA in a solvent such as DCM to give compound (9). The amide coupling between compound (9) and compound (4) using EDC and HOBt in a solvent such as DCM to give compound (10) is shown in step E. Step F shows the deprotection of compound (10) with tetrakis(triphenylphosphine) palladium and PhSiH₃ in a solvent such as DCM to give compound (11). Step F depicts the coupling of compound (11) with NHS using EDC in a solvent such as DCM to give compound (12).

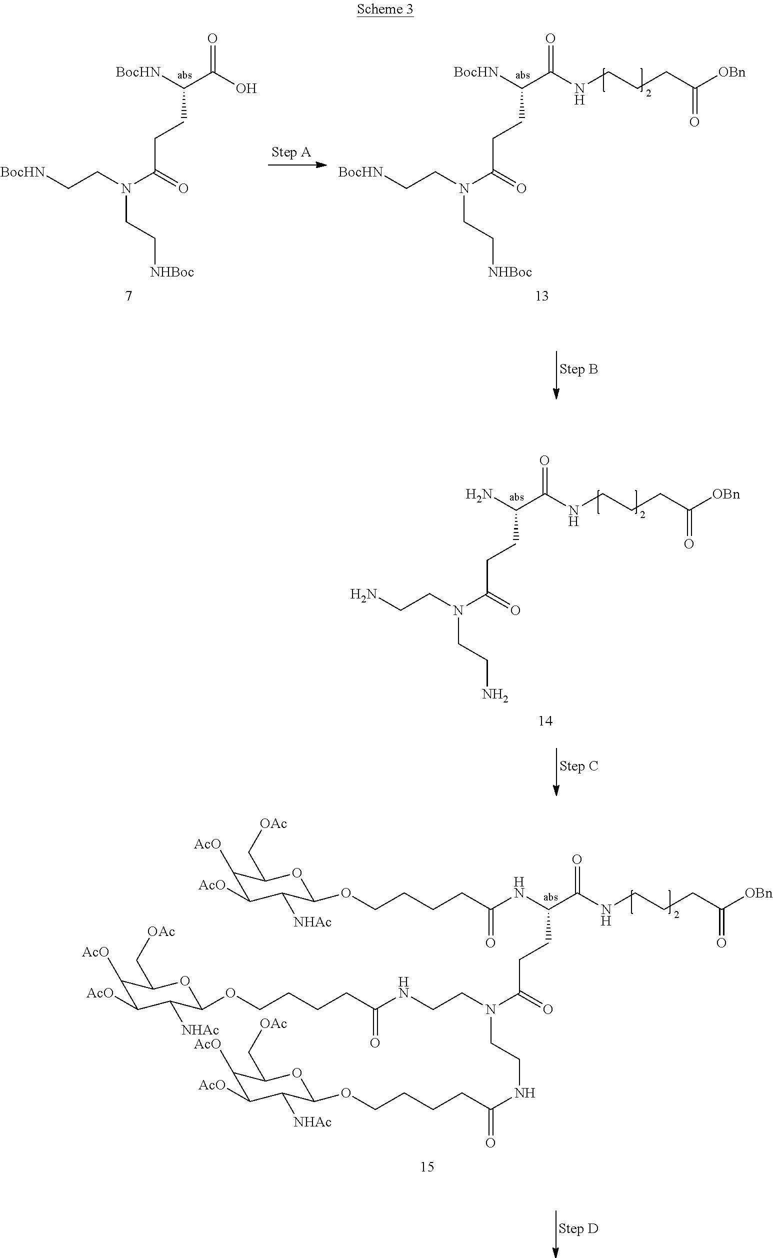

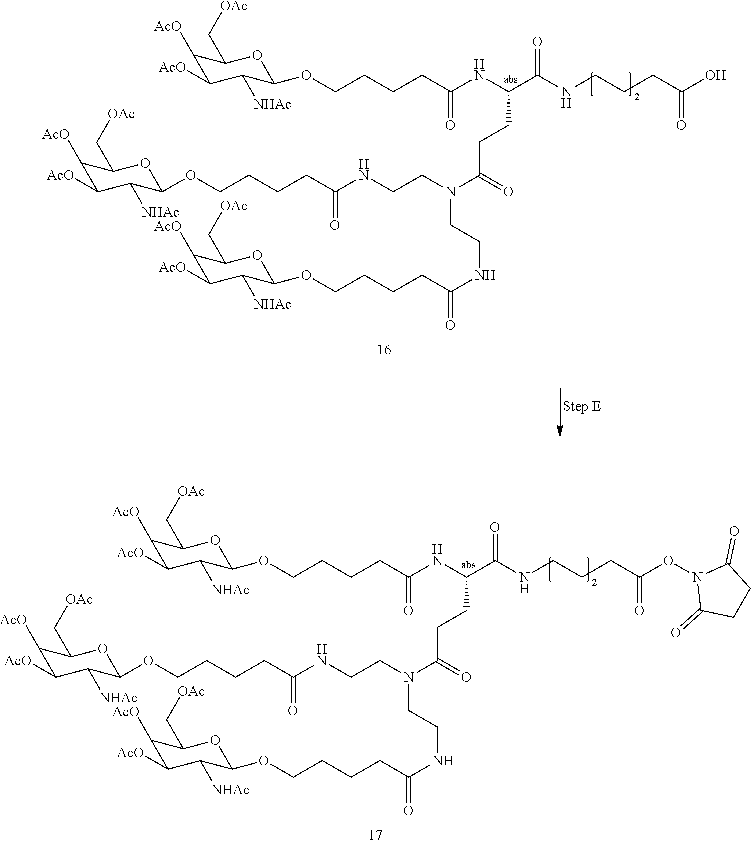

Scheme 3, steps A-C are essentially analogous to those of scheme 2, steps C-E beginning with compound (7) to give compounds (13), (14), and (15). Step D depicts the hydrogenation of compound (15) using palladium on carbon in a solvent such as MeOH to give compound (16). Step E is essentially analogous to the preparation of scheme 2, step G to give compound (17).

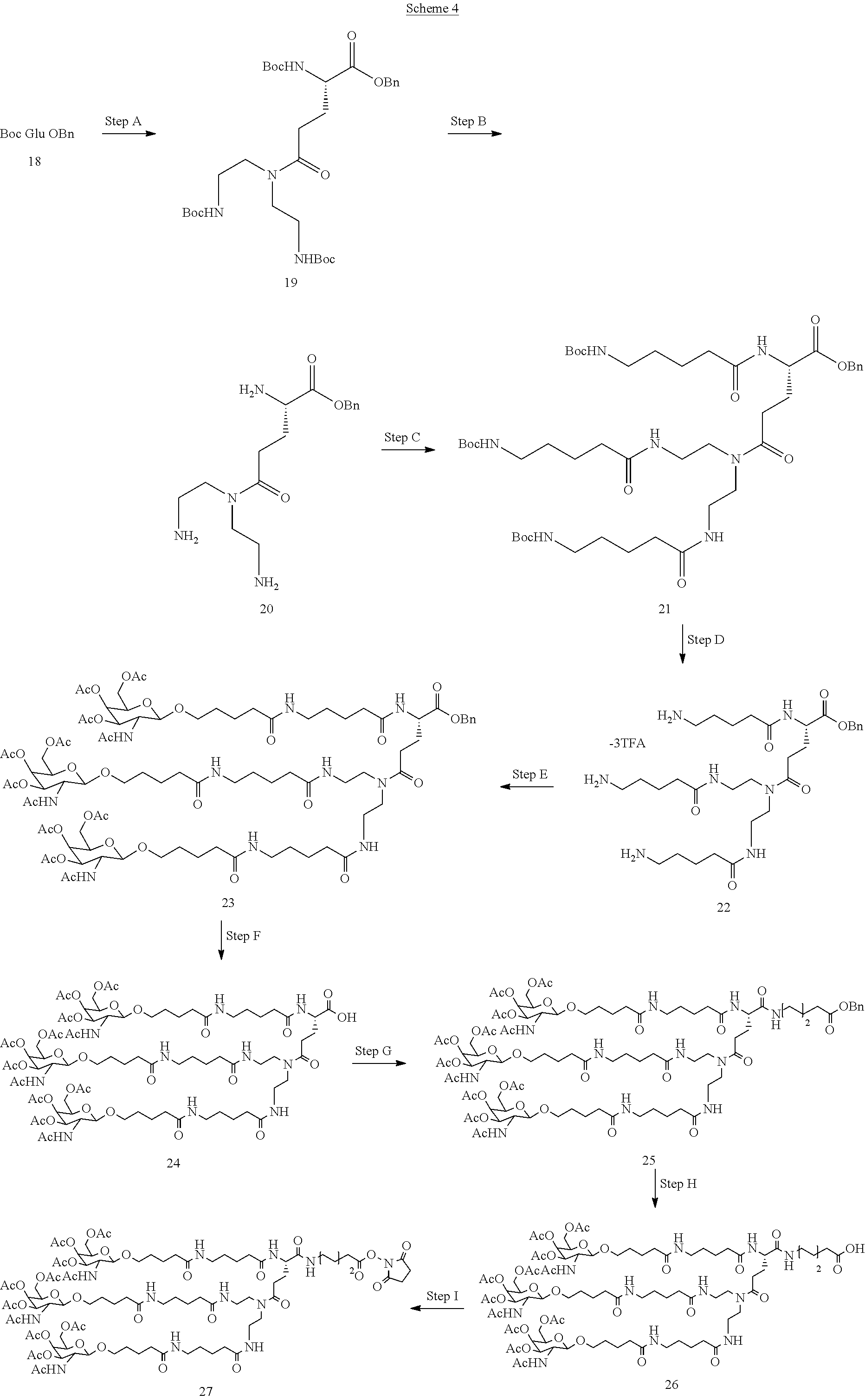

Scheme 4, steps A-I, are composed of a series of amide couplings and deprotections using methods essentially analogous to those found in schemes 2 and 3 beginning with compound (18) to give compound (27).

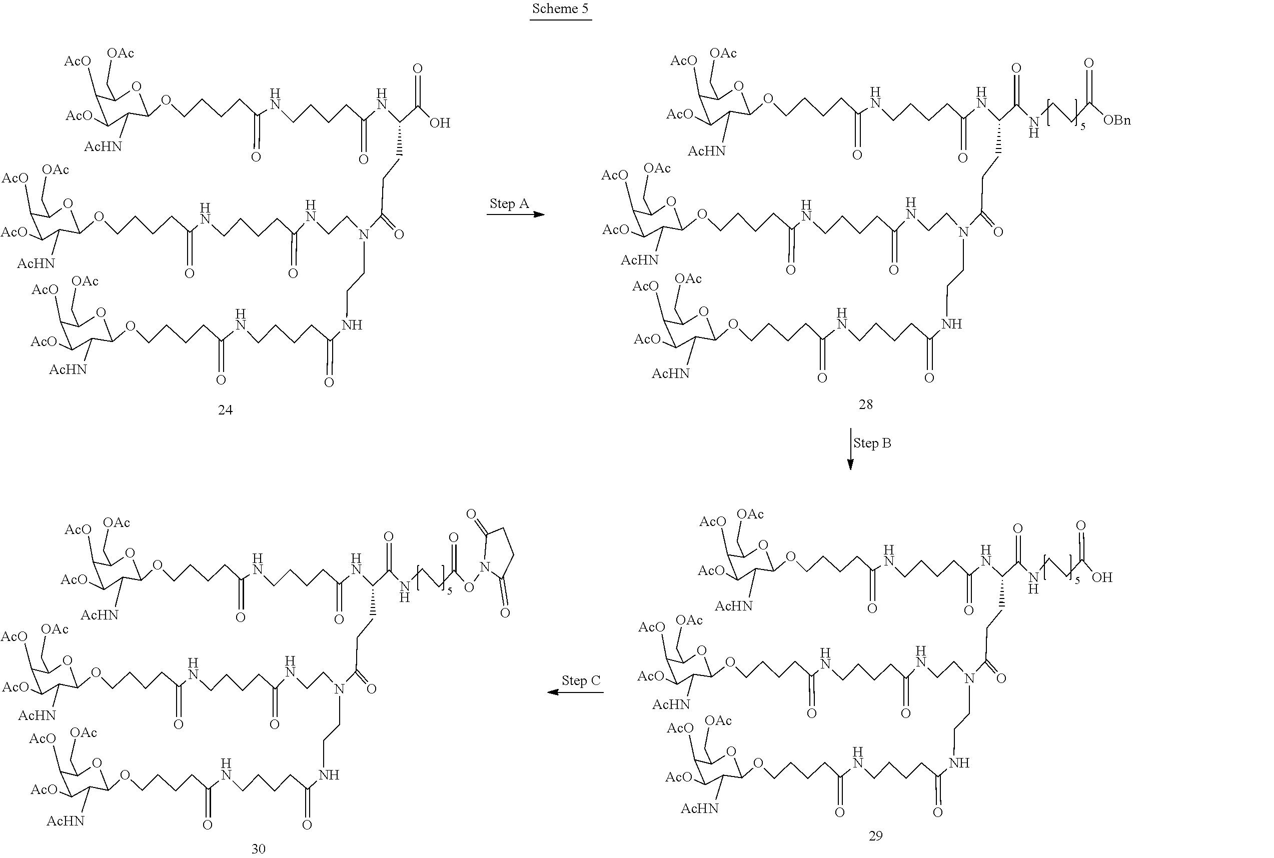

Scheme 5, steps A-C depict methods essentially analogous to those found in scheme 4, steps G-I beginning with compound (24) to give compound (30).

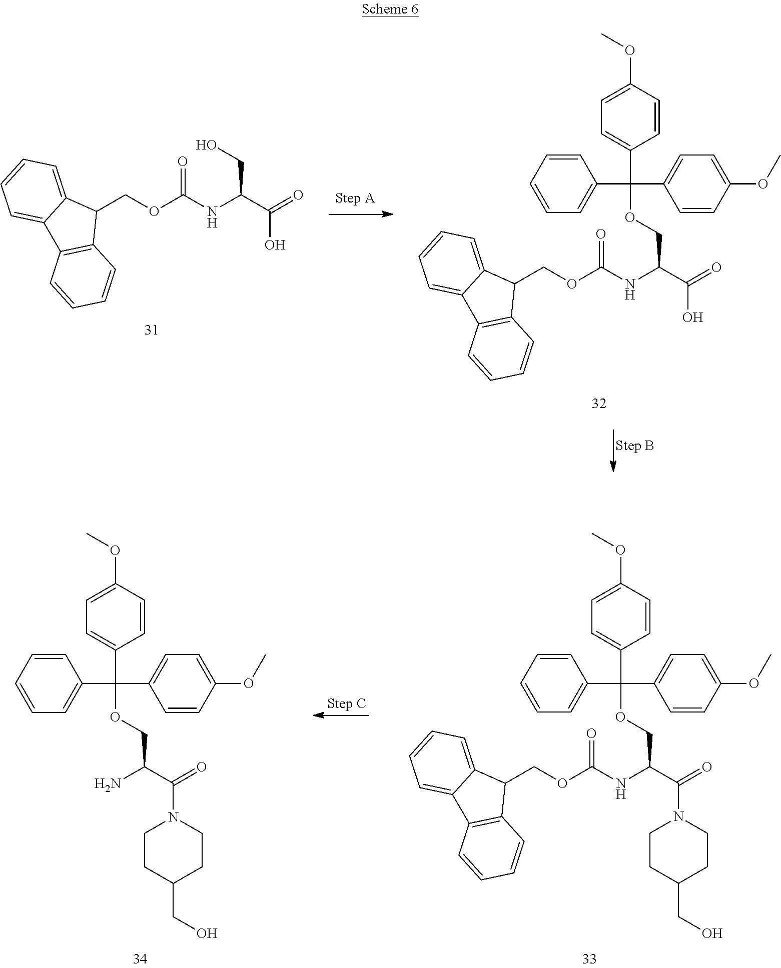 [SMILES: O=C(N[C@@H](CO)C(=O)O)OCC1c2ccccc2-c2ccccc21 ; COc1ccc(C(OC[C@H](NC(=O)OCC2c3ccccc3-c3ccccc32)C(=O)O)(c2ccccc2)c2ccc(OC)cc2)cc1 ; COc1ccc(C(OC[C@H](N)C(=O)N2CCC(CO)CC2)(c2ccccc2)c2ccc(OC)cc2)cc1 ; COc1ccc(C(OC[C@H](NC(=O)OCC2c3ccccc3-c3ccccc32)C(=O)N2CCC(CO)CC2)(c2ccccc2)c2ccc(OC)cc2)cc1]

Scheme 6, step A depicts the protection of compound (31) using DMTCl with a suitable base such as DIEA in a solvent such as DCM to give compound (32). Step B shows an amide coupling between compound (32) and piperidin-4-yl methanol using HBTU and HOBt with TMP in a solvent such as DCM to give compound (33). The deprotection of compound (33) with 20% piperidine in DMF to give compound (34) is shown in step C.

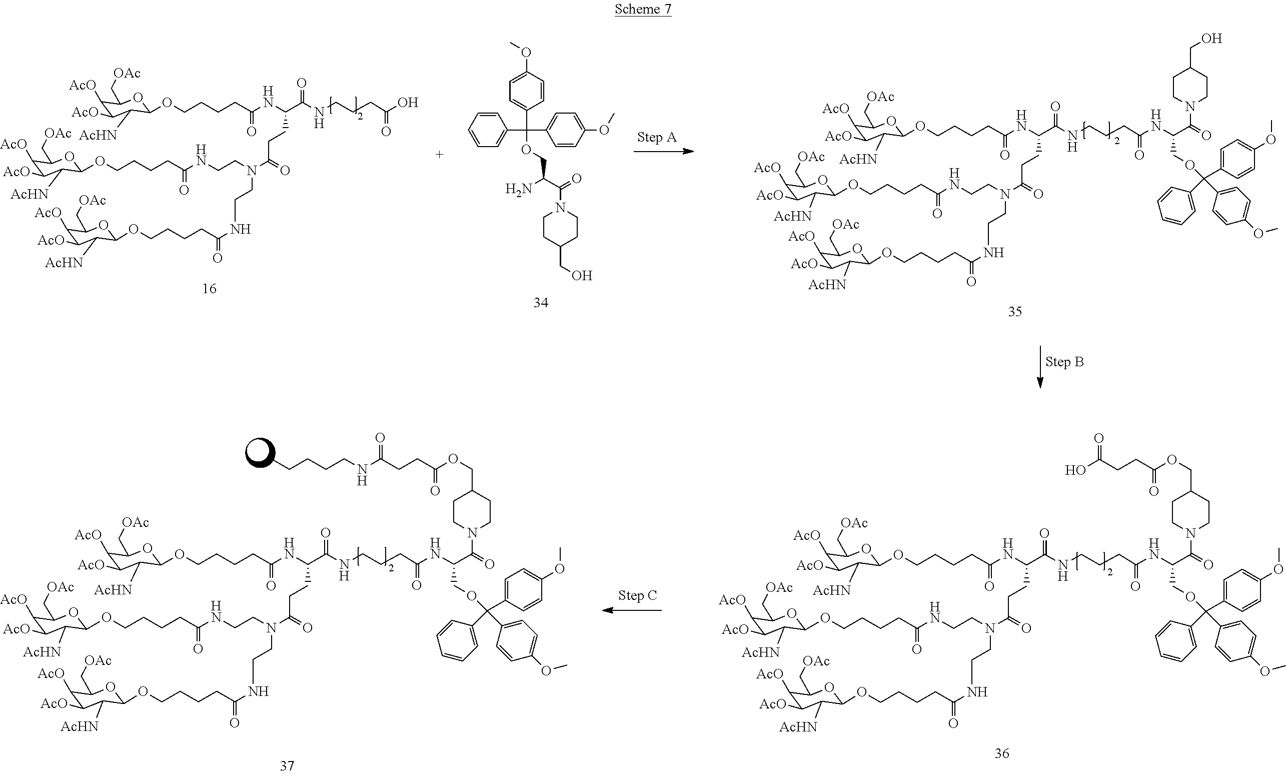

Scheme 7, step A is essentially analogous to scheme 2, step A to give compound (35) from the coupling of compounds (16) and (34). Step B shows the formation of compound (36) by adding succinic anhydride to compound (35) in an appropriate solvent such as DCM with a base system of TEA and DMAP. Step C depicts the loading of compound (36) onto resin with 2-(1H-benzotriazol-1-yl)-1,1,3,3-tetramethyluronium hexafluorophosphate and a base such as DIEA in a solvent system such as MeCN and DCM to give compound (37).

### Preparation 1

### (6,7-Diacetoxy-2-methyl-5,6,7,7a-tetrahydro-3aH-pyrano[3,2-d]oxazol-5-yl)methyl acetate

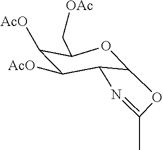 [SMILES: CC(=O)OCC1OC2OC(C)=NC2C(OC(C)=O)C1OC(C)=O]

To a solution of (5-acetamido-3,4,6-triacetoxy-tetrahydropyran-2-yl)methyl acetate (9.00 g, 23.1 mmol) in 1,2-DCE (46 mL) is added trimethylsilyl trifluoromethanesulfonate (6.5 mL, 35 mmol). The mixture is heated to 50° C. and stirred for 18 hours. After this time, the mixture is diluted with DCM (200 mL), washed with saturated NaHCO₃ (200 mL), and saturated aqueous sodium chloride solution (200 mL), dried over sodium sulfate, filtered, and concentrated in vacuo. The resulting residue is purified by silica gel flash chromatography eluting with 0-10% MeOH/DCM to give the title compound (6.434 g, 84%). ES/MS m/z 330 (M+H).

### Preparation 2

### (5-Acetamido-3,4-diacetoxy-6-hex-5-enoxy-tetrahydropyran-2-yl)methyl Acetate

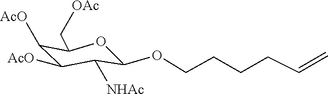 [SMILES: C=CCCCCOC1OC(COC(C)=O)C(OC(C)=O)C(OC(C)=O)C1NC(C)=O]

To a solution of (6,7-diacetoxy-2-methyl-5,6,7,7a-tetrahydro-3aH-pyrano[3,2-d]oxazol-5-yl)methyl acetate (30.43 g, 92.42 mmol) in 1,2-DCE (231 mL) is added hex-5-en-1-ol (22.2 mL, 185 mmol) followed by activated powdered 4 Å molecular sieves (15.6 g). The suspension is stirred at ambient temperature for 30 minutes and trimethylsilyl trifluoromethanesulfonate (19 mL, 101.9 mmol) is then added. The mixture is stirred at ambient temperature for 18 hours. After this time, the solution is filtered through diatomaceous earth and concentrated in vacuo. The resulting residue is purified by silica gel flash chromatography eluting with 30-100% EtOAc/hexanes to give the title compound (34.76 g, 86%). ES/MS m/z 430.4 (M+H).

### Preparation 3

### 5-[3-Acetamido-4,5-diacetoxy-6-(acetoxymethyl)tetrahydropyran-2-yl]oxypentanoic Acid

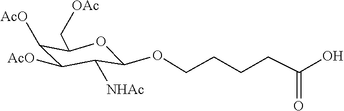 [SMILES: CC(=O)NC1C(OCCCCC(=O)O)OC(COC(C)=O)C(OC(C)=O)C1OC(C)=O]

A solution of (5-acetamido-3,4-diacetoxy-6-hex-5-enoxy-tetrahydropyran-2-yl)methyl acetate (34.76 g, 80.93 mmol) in MeCN (174 mL) and DCM (174 mL) is cooled to 0° C. A solution of sodium periodate (22.4 g, 104.7 mmol) is added and stirring is continued at 0° C. for 10 minutes. After this time, ruthenium (III) chloride (270 mg, 1.3 mmol) is added and the mixture is stirred while warming to ambient temperature. After stirring for 2 hours, additional sodium periodate (66 g, 308.4 mmol) is added and stirring is continued for 18 hours. After this time, the mixture is extracted with 3:1 CH₃Cl:IPA (2×500 mL), washed with saturated aqueous sodium chloride solution (1 L), dried over sodium sulfate, filtered, and concentrated in vacuo. The resulting residue is purified by silica gel flash chromatography eluting with 0-40% MeOH/DCM to give the title compound (29.75 g, 82%). ES/MS m/z 448.4 (M+H).

### Preparation 4

### Benzyl 6-aminohexanoate Hydrochloride

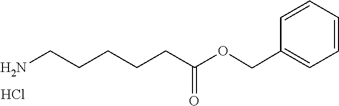 [SMILES: Cl ; NCCCCCC(=O)OCc1ccccc1]

To a suspension of 6-aminohexanoic acid (5.00 g, 38.1 mmol) in THF (38 mL) is added benzyl alcohol (47 mL, 453.7 mmol) and the mixture is cooled to 0° C. Thionyl chloride (8.6 mL, 120 mmol) is added dropwise and the mixture is stirred for 18 hours while warming to ambient temperature. After this time, ether (166 mL) is added and the reaction vessel is transferred to a freezer at −20° C. for 1 hour. After this time, the solid precipitate is collected by filtration to give the title compound (8.57 g, 81%). ES/MS m/z 222 (M+H).

### Preparation 5

### Benzyl 11-aminoundecanoate Hydrochloride

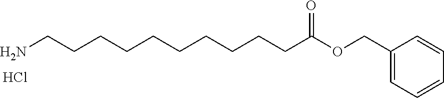 [SMILES: Cl ; NCCCCCCCCCCC(=O)OCc1ccccc1]

The title compound is prepared from 11-aminoundecanoic acid in a manner essentially analogous to the method of preparation 4. ES/MS m/z 292.2 (M+H).

### Preparation 6

### Allyl 11-aminoundecanoate Hydrochloride

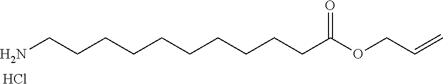 [SMILES: C=CCOC(=O)CCCCCCCCCCN ; Cl]

A vessel is charged with 11-aminoundecanoic acid (9.00 g, 44.7 mmol) in allyl alcohol (42 mL) and the mixture is cooled to 0° C. Thionyl chloride (6.5 mL, 89.4 mmol) is added and the mixture is stirred for 18 hours while warming to ambient temperature. After this time, the mixture is concentrated in vacuo and ether (200 mL) is added to the residue to obtain a white suspension. The mixture is stirred at ambient temperature for 10 minutes and the solid precipitate is collected by filtration to obtain the product (12.0 g, 97%). ES/MS m/z 242.2 (M+H).

### Preparation 7

### (2S)-3-[Bis(4-methoxyphenyl)-phenyl-methoxy]-2-(9H-fluoren-9ylmethoxycarbonylamino) propanoic Acid

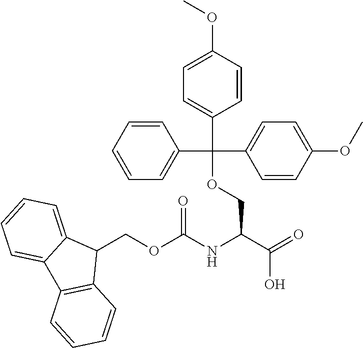 [SMILES: COc1ccc(C(OC[C@H](NC(=O)OCC2c3ccccc3-c3ccccc32)C(=O)O)(c2ccccc2)c2ccc(OC)cc2)cc1]

To a stirring solution of (2S)-2-(9H-fluoren-9-ylmethoxycarbonylamino)-3-hydroxy-propanoic acid (40 g, 0.122 mol) in dry DCM (400 mL) is added DIEA (64 mL, 0.366 mol) at 0° C. under inert atmosphere. To this, a solution of DMTCl (49.6 g, 0.146 mol) in DCM (200 mL) is added slowly. The resulting reaction mixture is brought to ambient temperature and stirred for 16 hours. After this time, the reaction mixture is diluted with water (12.5 vol) and extracted with DCM (25 vol). The organic layer is dried over anhydrous sodium sulphate, filtered, and concentrated in vacuo. The crude obtained is washed with 10% EtOAc/hexane (12.5 vol) and dried under vacuum to give the title compound as a pale brown solid (62 g, crude). This material was taken to next step without any further purification.

TLC: 5% MeOH/CH₂Cl₂ (R_{f}: 0.5) UV, 254 nM.

### Preparation 8

### 9H-Fluoren-9-ylmethyl N-[(1S)-1-[[bis(4-methoxyphenyl)-phenyl-methoxy]methyl]-2-[4-(hydroxymethyl)-1-piperidyl]-2-oxo-ethyl]carbamate

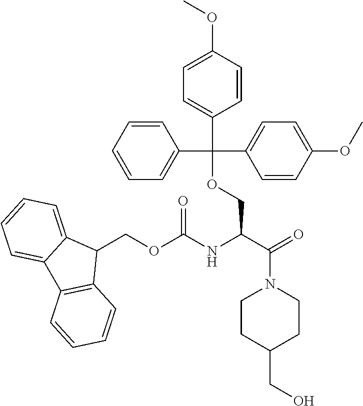 [SMILES: COc1ccc(C(OC[C@H](NC(=O)OCC2c3ccccc3-c3ccccc32)C(=O)N2CCC(CO)CC2)(c2ccccc2)c2ccc(OC)cc2)cc1]

To a stirring solution of (2S)-3-[bis(4-methoxyphenyl)-phenyl-methoxy]-2-(9H-fluoren-9ylmethoxycarbonylamino) propanoic acid (62 g, 0.103 mol) in DCM (750 mL) are added slowly HBTU (78.3 g, 0.206 mol), HOBt (27.9 g, 0.206 mol), and piperidin-4-yl methanol (15.4 g, 0.134 mol) followed by TMP (15 mL, 0.113 mol) at 0° C. under inert atmosphere. The resulting reaction mixture is brought to ambient temperature and stirred for 4 hours. After this time, the reaction mixture is diluted with water (8 vol) and extracted with DCM (15 vol). The organic layer is dried over anhydrous sodium sulphate, filtered, and concentrated in vacuo. The resulting residue is purified by silica gel flash chromatography eluting with 20-40% EtOAc/hexane and 1% MeOH/DCM to give the title compound (40 g, 52% over two steps). ¹H NMR (DMSO-d₆) δ 7.88 (br d, J=7.5 Hz, 2H), 7.79-7.59 (m, 3H), 7.45-7.12 (m, 13H), 6.92-6.76 (m, 4H), 4.79-4.44 (m, 2H), 4.32 (br d, J=11.4 Hz, 2H), 4.20 (br s, 2H), 3.71 (s, 6H), 3.21 (br s, 4H), 2.99-2.79 (m, 1H), 2.69 (br s, 2H), 1.81-1.43 (m, 3H), 1.08-0.73 (m, 2H).

### Preparation 9

### (2S)-2-Amino-3-[bis(4-methoxyphenyl)-phenyl-methoxy]-1-[4-(hydroxymethyl)-1-piperidyl]propan-1-one

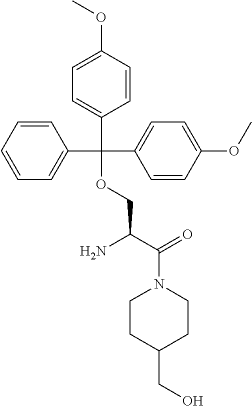 [SMILES: COc1ccc(C(OC[C@H](N)C(=O)N2CCC(CO)CC2)(c2ccccc2)c2ccc(OC)cc2)cc1]

A solution of 20% piperidine in DMF (400 mL) is added slowly to 9H-fluoren-9-ylmethyl N-[(1S)-1-[[bis(4-methoxyphenyl)-phenyl-methoxy]methyl]-2-[4-(hydroxymethyl)-1-piperidyl]-2-oxo-ethyl]carbamate (40 g, 0.055 mol) at 0° C. under inert atmosphere. The resulting reaction mixture is stirred at ambient temperature for 1 hour. After this time, the mixture is diluted with water (15 vol) and extracted with EtOAc (30 vol). The organic layer is dried over anhydrous sodium sulphate, filtered, and concentrated in vacuo. The resulting residue is purified by silica gel flash chromatography eluting with 1-8% MeOH/DCM to give the title compound as an off white solid (13 g, 47%). ES/MS m/z 1009.5 (2M+H).

### Preparation 10

### Methyl (2S)-5-[bis[2-(tert-butoxycarbonylamino)ethyl]amino]-2-(tert-butoxycarbonylamino)-5-oxo-pentanoate

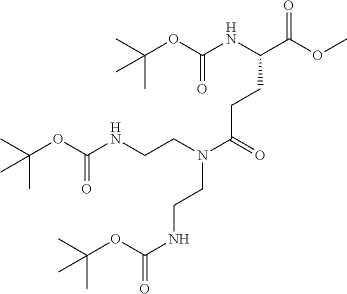 [SMILES: COC(=O)[C@H](CCC(=O)N(CCNC(=O)OC(C)(C)C)CCNC(=O)OC(C)(C)C)NC(=O)OC(C)(C)C]

To a flask containing(S)-4-((tert-butoxycarbonyl)amino)-5-methoxy-5-oxopentanoic acid (7.00 g, 26.8 mmol) and HOBt (4.16 g, 30.8 mmol) are added DMF (179 mL) and (2-(1H-benzotriazol-1-yl)-1,1,3,3-tetramethyluronium hexafluorophosphate (11.7 g, 30.9 mmol). DIEA (14 mL, 80.3 mmol) is added and the mixture is stirred at ambient temperature for 5 minutes. After this time, tert-butyl N-[2-[2-(tert-butoxycarbonylamino)ethylamino]ethyl]carbamate (8.94 g, 29.5 mmol) is added in one portion and stirring is continued at ambient temperature. After stirring for 18 hours, the mixture is diluted with EtOAc (400 mL), washed with water (2×400 mL) and saturated aqueous sodium chloride solution (400 mL), dried over sodium sulfate, filtered, and concentrated in vacuo. The resulting residue is purified by silica gel flash chromatography eluting with 40-100% EtOAc/hexanes to give the title compound (13.01 g, 89%). ES/MS m/z 547.40 (M+H).

### Preparation 11

### (2S)-5-[Bis[2-(tert-butoxycarbonylamino)ethyl]amino]-2-(tert-butoxycarbonylamino)-5-oxo-pentanoic Acid

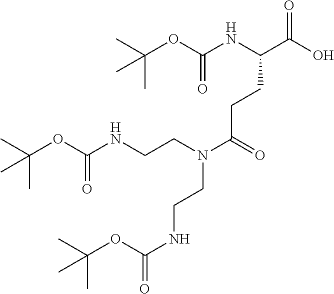 [SMILES: CC(C)(C)OC(=O)NCCN(CCNC(=O)OC(C)(C)C)C(=O)CC[C@H](NC(=O)OC(C)(C)C)C(=O)O]

A flask is charged with methyl (2S)-5-[bis[2-(tert-butoxycarbonylamino)ethyl]amino]-2-(tert-butoxycarbonylamino)-5-oxo-pentanoate (13.01 g, 23.8 mmol), THF (120 mL), and MeOH (120 mL). IN NaOH (71 mL, 71 mmol) is added and the mixture is stirred at ambient temperature. After 1 hour, the mixture is concentrated in vacuo and redissolved in water (300 mL). 5N HCl (12 mL) is added to bring the pH to 4. The mixture is extracted with DCM (3×300 mL) and the combined organic layers are washed with saturated aqueous sodium chloride solution (1 L), dried over sodium sulfate, filtered, and concentrated to give the title compound (12.41 g, 98%). ES/MS m/z 531.60 (M−H).

### Preparation 12

### Allyl 11-[[(2S)-5-[bis[2-(tert-butoxycarbonylamino)ethyl]amino]-2-(tert-butoxycarbonylamino)-5-oxo-pentanoyl]amino]undecanoate

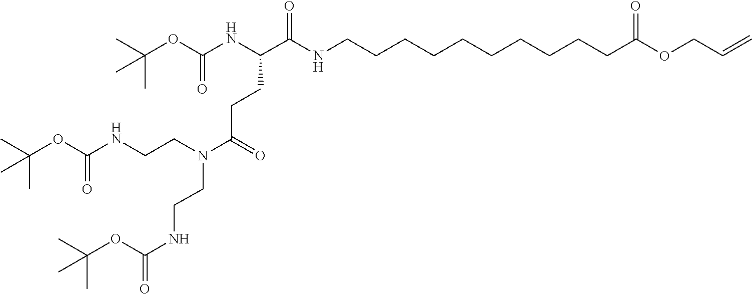 [SMILES: C=CCOC(=O)CCCCCCCCCCNC(=O)[C@H](CCC(=O)N(CCNC(=O)OC(C)(C)C)CCNC(=O)OC(C)(C)C)NC(=O)OC(C)(C)C]

To a flask containing (2S)-5-[bis[2-(tert-butoxycarbonylamino)ethyl]amino]-2-(tert-butoxycarbonylamino)-5-oxo-pentanoic acid (500 mg, 0.94 mmol) and allyl 11-aminoundecanoate hydrochloride (313 mg, 1.13 mmol) is added DMF (6.25 mL) and (1-[bis(dimethylamino)methylene]-1H-1,2,3-triazolo[4,5-b]pyridinium 3-oxide hexafluorophosphate (428 mg, 1.12 mmol). Following addition of DIEA (0.5 mL, 3 mmol) the mixture is stirred at ambient temperature for 18 hours. After this time, the mixture is diluted with EtOAc (200 mL), washed with water (3×200 mL) and saturated aqueous sodium chloride solution (200 mL), dried over sodium sulfate, filtered, and concentrated in vacuo. The resulting residue is purified by silica gel flash chromatography eluting with 40-100% EtOAc/hexanes to give the title compound (687 mg, 97%). ¹H NMR (DMSO-d6) δ 7.78-7.64 (m, 1H), 6.98-6.7 (m, 2H), 5.96-5.84 (m, 1H), 5.31-5.25 (m, 1H), 5.23-5.17 (m, 1H), 4.56-4.50 (m, 2H), 3.88-3.67 (m, 1H), 3.30-3.19 (m, 4H), 3.11-2.91 (m, 6H), 2.35-2.12 (m, 4H), 1.88-1.65 (m, 2H), 1.58-1.47 (m, 2H), 1.46-1.30 (m, 30H), 1.30-1.18 (m, 12H).

### Preparation 13

### Allyl (S)-11-(2-amino-5-(bis(2-aminoethyl)amino)-5-oxopentanamido)undecanoate

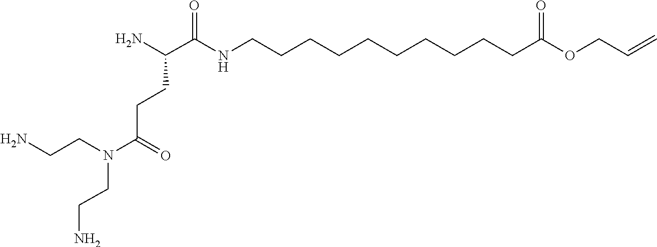 [SMILES: C=CCOC(=O)CCCCCCCCCCNC(=O)[C@@H](N)CCC(=O)N(CCN)CCN]

To a solution of allyl 11-[[(2S)-5-[bis[2-(tert-butoxycarbonylamino)ethyl]amino]-2-(tert-butoxycarbonylamino)-5-oxo-pentanoyl]amino]undecanoate (687 mg, 0.91 mmol) in DCM (15 mL) is added TFA (15 mL). The mixture is stirred at ambient temperature. After 1.5 hours, the mixture is concentrated in vacuo. The residue is taken up in MeOH and applied to an ion exchange cartridge. The cartridge is eluted with MeOH (150 mL) followed by 7N NH₃/MeOH (150 mL). The basic fraction is concentrated in vacuo to give the title compound (410 mg, 99%). ES/MS m/z 456.4 (M+H).

### Preparation 14

### Allyl 11-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]undecanoate

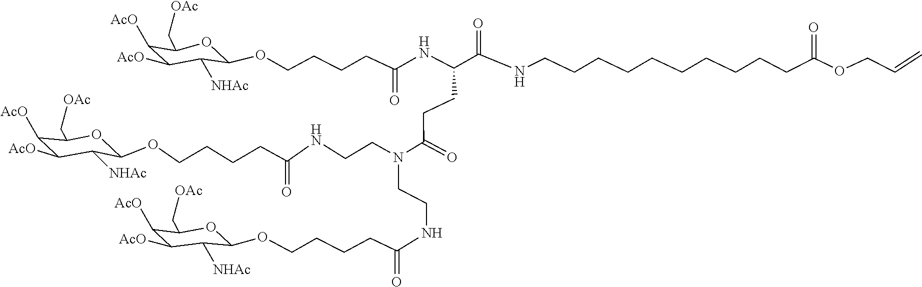

A flask is charged with 5-[3-acetamido-4,5-diacetoxy-6-(acetoxymethyl)tetrahydropyran-2-yl]oxypentanoic acid (489 mg, 1.09 mmol) and allyl (S)-11-(2-amino-5-(bis(2-aminoethyl)amino)-5-oxopentanamido)undecanoate (150 mg, 0.33 mmol). DCM (3.35 mL) is added followed by 1-hydroxybenzotriazole monohydrate (164 mg, 1.07 mmol) and 1-(3-dimethylaminopropyl)-3-ethylcarbodiimide hydrochloride (206 mg, 1.07 mmol). The mixture is stirred at ambient temperature for 18 hours. After this time, the solution is diluted with EtOAc (100 mL), washed with saturated NaHCO₃ (2×100 mL), saturated aqueous NH₄Cl (100 mL), and saturated aqueous sodium chloride solution (100 mL). The organic layer is dried over sodium sulfate, filtered, and concentrated in vacuo. The resulting residue is purified by silica gel flash chromatography eluting with 0-10% MeOH/DCM to give the title compound (424 mg, 74%). ES/MS m/z 872.80 (M+2H)/2.

### Preparation 15

### 11-[[(2S)-2-[5-[3-Acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]undecanoic Acid

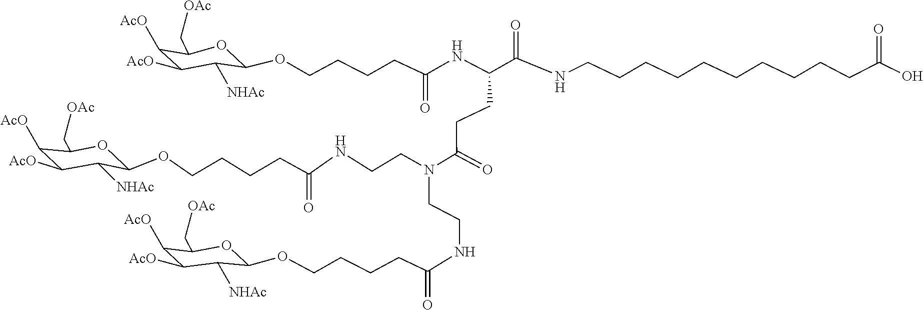

To a solution of allyl 11-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]undecanoate (354 mg, 0.20 mmol) in DCM (2 mL) is added tetrakis(triphenylphosphine) palladium (29 mg, 0.02 mmol) followed by PhSiH₃ (51 uL, 0.41 mmol). The mixture is stirred at ambient temperature for 2 hours, after which it is diluted with saturated aqueous NaHCO₃ (100 mL). IN NaOH (15 mL) is added to bring the pH to about 10. The aqueous solution is washed with DCM (3×100 mL) and then acidified with concentrated HCl (5 mL) and then aqueous 5N HCl (15 mL). The aqueous layer is extracted with DCM (100 mL) and the organic layer is dried over sodium sulfate, filtered, and concentrated in vacuo. The resulting residue is purified by silica gel flash chromatography eluting with 0-20% MeOH/DCM to give the title compound (151 mg, 44%). ES/MS m/z 852.60 (M+2H)/2.

### Preparation 16

### (2,5-Dioxopyrrolidin-1-yl) 11-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]undecanoate

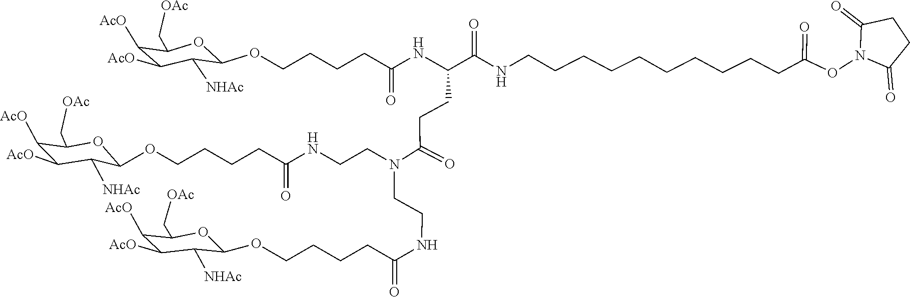 [SMILES: CC(=O)NC1C(OCCCCC(=O)NCCN(CCNC(=O)CCCCOC2OC(COC(C)=O)C(OC(C)=O)C(OC(C)=O)C2NC(C)=O)C(=O)CC[C@H](NC(=O)CCCCOC2OC(COC(C)=O)C(OC(C)=O)C(OC(C)=O)C2NC(C)=O)C(=O)NCCCCCCCCCCC(=O)ON2C(=O)CCC2=O)OC(COC(C)=O)C(OC(C)=O)C1OC(C)=O]

To a reaction vial are added 11-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]undecanoic acid (50 mg, 0.03 mmol), N-hydroxysuccinimide (5 mg, 0.04 mmol), and 1-(3-dimethylaminopropyl)-3-ethylcarbodiimide hydrochloride (8 mg, 0.04 mmol). DCM (0.3 mL) is added and the mixture is stirred at ambient temperature. After 18 hours, the mixture is loaded directly onto a silica gel cartridge and the crude mixture is purified by silica gel flash chromatography eluting with 0-10% MeOH/DCM to give the title compound (49 mg, 93%). ES/MS m/z 901.40 (M+2H)/2.

### Preparation 17

### Benzyl 6-[[(2S)-5-[bis[2-(tert-butoxycarbonylamino)ethyl]amino]-2-(tert-butoxycarbonylamino)-5-oxo-pentanoyl]amino]hexanoate

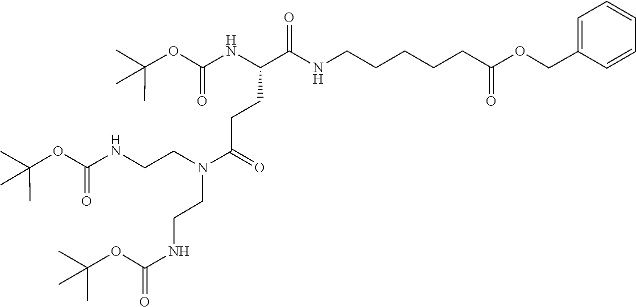 [SMILES: CC(C)(C)OC(=O)NCCN(CCNC(=O)OC(C)(C)C)C(=O)CC[C@H](NC(=O)OC(C)(C)C)C(=O)NCCCCCC(=O)OCc1ccccc1]

The title compound is prepared from (2S)-5-[bis[2-(tert-butoxycarbonylamino)ethyl]amino]-2-(tert-butoxycarbonylamino)-5-oxo-pentanoic acid and benzyl 6-aminohexanoate hydrochloride in a manner essentially analogous to the method of preparation 10. ES/MS m/z 736.40 (M+H).

### Preparation 18

### Benzyl 6-[[(2S)-2-amino-5-[bis(2-aminoethyl)amino]-5-oxo-pentanoyl]amino]hexanoate tris(trifluoroacetic Acid)

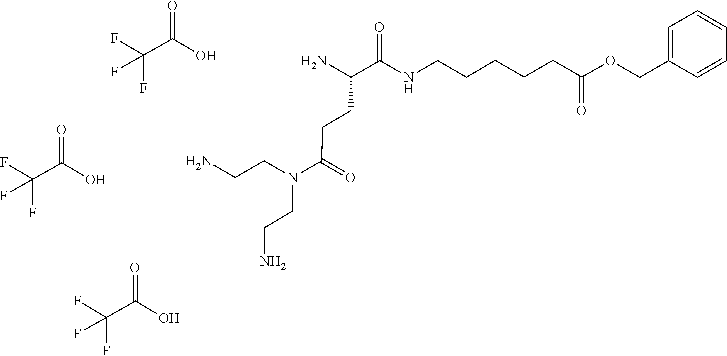 [SMILES: O=C(O)C(F)(F)F ; O=C(O)C(F)(F)F ; NCCN(CCN)C(=O)CC[C@H](N)C(=O)NCCCCCC(=O)OCc1ccccc1 ; O=C(O)C(F)(F)F]

To a solution of benzyl 6-[[(2S)-5-[bis[2-(tert-butoxycarbonylamino)ethyl]amino]-2-(tert-butoxycarbonylamino)-5-oxo-pentanoyl]amino]hexanoate (15.47 g, 21.02 mmol) in DCM (105 mL) is added TFA (16 mL, 210.2 mmol). The mixture is stirred at ambient temperature for 24 hours. After this time, additional TFA (16 mL, 210.2 mmol) is added and stirring is continued for an additional 2 hours. After this time, the mixture is concentrated in vacuo. The resulting residue is azeotroped with toluene (2×30 mL). The resulting oil is further dried in a vacuum oven at 40° C. for 4 hours to give the title compound (28.08 g, 58% purity accounting for residual toluene, 99+%). ES/MS m/z 436.40 (M+H). The compound is dissolved in 70 mL DMF to make a 0.3M solution that is used in the next step.

### Preparation 19

### Benzyl 6-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoate

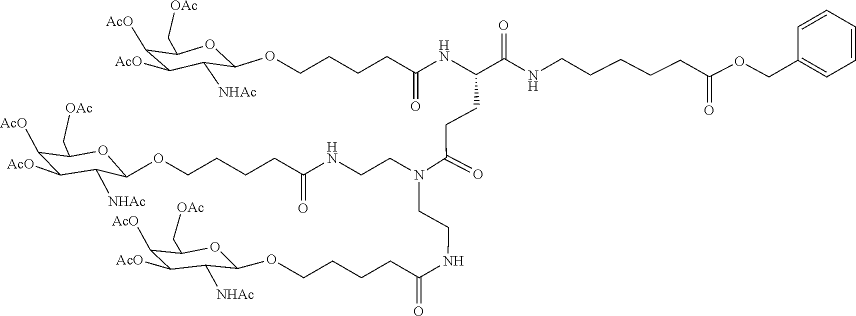 [SMILES: CC(=O)NC1C(OCCCCC(=O)NCCN(CCNC(=O)CCCCOC2OC(COC(C)=O)C(OC(C)=O)C(OC(C)=O)C2NC(C)=O)C(=O)CC[C@H](NC(=O)CCCCOC2OC(COC(C)=O)C(OC(C)=O)C(OC(C)=O)C2NC(C)=O)C(=O)NCCCCCC(=O)OCc2ccccc2)OC(COC(C)=O)C(OC(C)=O)C1OC(C)=O]

The title compound is prepared from 5-[3-acetamido-4,5-diacetoxy-6-(acetoxymethyl)tetrahydropyran-2-yl]oxypentanoic acid and benzyl 6-[[(2S)-2-amino-5-[bis(2-aminoethyl)amino]-5-oxo-pentanoyl]amino]hexanoate tris trifluoroacetic acid and in a manner essentially analogous to the method of preparation 10. ES/MS m/z 862 (M+2H)/2.

### Preparation 20

### 6-[[(2S)-2-[5-[3-Acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoic Acid

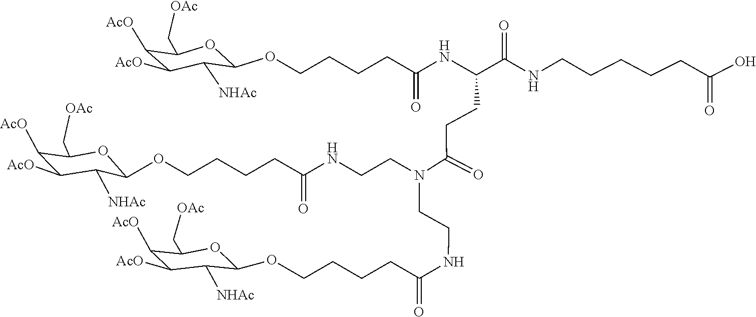 [SMILES: CC(=O)NC1C(OCCCCC(=O)NCCN(CCNC(=O)CCCCOC2OC(COC(C)=O)C(OC(C)=O)C(OC(C)=O)C2NC(C)=O)C(=O)CC[C@H](NC(=O)CCCCOC2OC(COC(C)=O)C(OC(C)=O)C(OC(C)=O)C2NC(C)=O)C(=O)NCCCCCC(=O)O)OC(COC(C)=O)C(OC(C)=O)C1OC(C)=O]

Palladium on carbon (1.90 g, 0.89 mmol, 5 mass %, 50% wet) is placed in a round-bottom flask and the vessel is evacuated and backfilled with nitrogen three times. A solution of benzyl 6-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoate (15.41 g, 8.94 mmol) in MeOH (178 mL) is added via syringe. The flask is evacuated and backfilled with 1 atm hydrogen and the mixture is stirred at ambient temperature under 1 atm hydrogen for 18 hours. After this time, the mixture is filtered through diatomaceous earth and the filtrate is concentrated in vacuo to give the title compound (13.85 g, 95%). ES/MS m/z 817.2 (M+2H)/2.

### Preparation 21

### (2,5-Dioxopyrrolidin-1-yl) 6-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoate

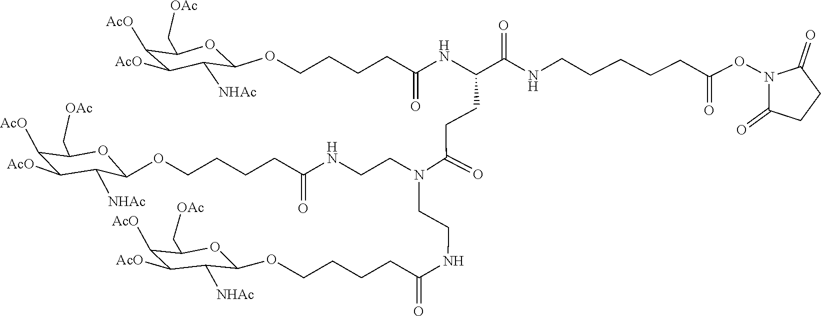 [SMILES: CC(=O)NC1C(OCCCCC(=O)NCCN(CCNC(=O)CCCCOC2OC(COC(C)=O)C(OC(C)=O)C(OC(C)=O)C2NC(C)=O)C(=O)CC[C@H](NC(=O)CCCCOC2OC(COC(C)=O)C(OC(C)=O)C(OC(C)=O)C2NC(C)=O)C(=O)NCCCCCC(=O)ON2C(=O)CCC2=O)OC(COC(C)=O)C(OC(C)=O)C1OC(C)=O]

The title compound is prepared from 6-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoic acid in a manner essentially analogous to the method of preparation 16. ES/MS m/z 866.20 (M+2H)/2.

### Preparation 22

### Benzyl (2S)-5-[bis[2-(tert-butoxycarbonylamino)ethyl]amino]-2-(tert-butoxycarbonylamino)-5-oxo-pentanoate

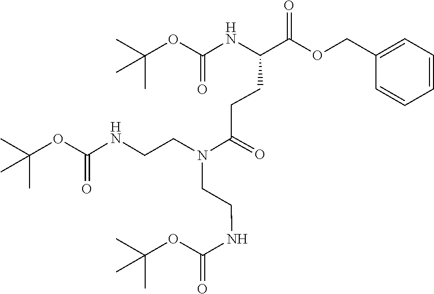 [SMILES: CC(C)(C)OC(=O)NCCN(CCNC(=O)OC(C)(C)C)C(=O)CC[C@H](NC(=O)OC(C)(C)C)C(=O)OCc1ccccc1]

The title compound is prepared from tert-butyl N-[2-[2-(tert-butoxycarbonylamino)ethylamino]ethyl]carbamate and (4S)-5-benzyloxy-4-(tert-butoxycarbonylamino)-5-oxo-pentanoic acid in a manner essentially analogous to the method of preparation 12. ES/MS m/z 623.6 (M+H).

### Preparation 23

### Benzyl (2S)-2-amino-5-[bis(2-aminoethyl)amino]-5-oxo-pentanoate tris(trifluoroacetic Acid) Salt

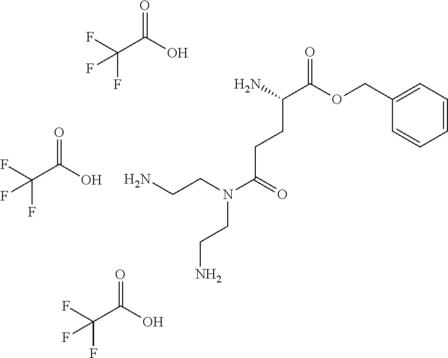 [SMILES: O=C(O)C(F)(F)F ; O=C(O)C(F)(F)F ; NCCN(CCN)C(=O)CC[C@H](N)C(=O)OCc1ccccc1 ; O=C(O)C(F)(F)F]

The title compound is prepared from benzyl (2S)-5-[bis[2-(tert-butoxycarbonylamino)ethyl]amino]-2-(tert-butoxycarbonylamino)-5-oxo-pentanoate in a manner essentially analogous to the method of preparation 18. ES/MS m/z 323.2 (M+H).

### Preparation 24

### Benzyl (2S)-5-[bis[2-[5-(tert-butoxycarbonylamino)pentanoylamino]ethyl]amino]-2-[5-(tert-butoxycarbonylamino)pentanoylamino]-5-oxo-pentanoate

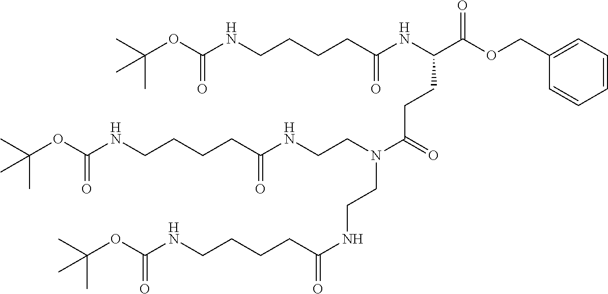 [SMILES: CC(C)(C)OC(=O)NCCCCC(=O)NCCN(CCNC(=O)CCCCNC(=O)OC(C)(C)C)C(=O)CC[C@H](NC(=O)CCCCNC(=O)OC(C)(C)C)C(=O)OCc1ccccc1]

The title compound is prepared from 5-(tert-butoxycarbonylamino)pentanoic acid and benzyl (2S)-2-amino-5-[bis(2-aminoethyl)amino]-5-oxo-pentanoate tris(trifluoroacetic acid) salt in a manner essentially analogous to the method of preparation 10. ES/MS m/z 920.6 (M+H).

### Preparation 25

### Benzyl (2S)-2-(5-aminopentanoylamino)-5-[bis[2-(5-aminopentanoylamino)ethyl]amino]-5-oxo-pentanoate tris(trifluoroacetic Acid) Salt

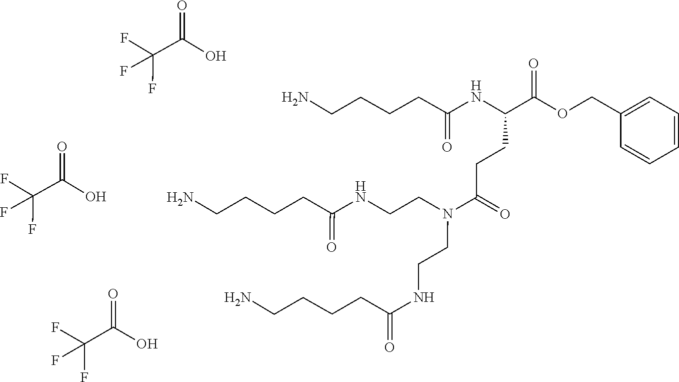 [SMILES: O=C(O)C(F)(F)F ; O=C(O)C(F)(F)F ; O=C(O)C(F)(F)F ; NCCCCC(=O)NCCN(CCNC(=O)CCCCN)C(=O)CC[C@H](NC(=O)CCCCN)C(=O)OCc1ccccc1]

The title compound is prepared from benzyl (2S)-5-[bis[2-[5-(tert-butoxycarbonylamino)pentanoylamino]ethyl]amino]-2-[5-(tert-butoxycarbonylamino)pentanoylamino]-5-oxo-pentanoate in a manner essentially analogous to the method of preparation 18. ES/MS m/z 620.4 (M+H).

### Preparation 26

### Benzyl (2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoate

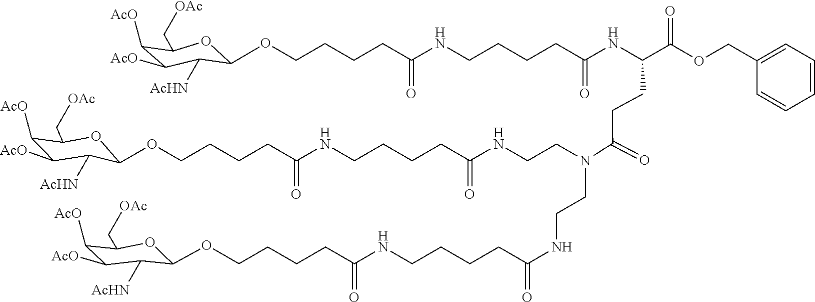 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)OCc2ccccc2)OC(COC(C)=O)C(OC(C)=O)C1OC(C)=O]

The title compound is prepared from 5-[3-acetamido-4,5-diacetoxy-6-(acetoxymethyl)tetrahydropyran-2-yl]oxypentanoic acid and benzyl (2S)-2-(5-aminopentanoylamino)-5-[bis[2-(5-aminopentanoylamino)ethyl]amino]-5-oxo-pentanoate tris(trifluoroacetic acid) salt and in a manner essentially analogous to the method of preparation 10. ES/MS m/z 954.80 (M+2H)/2.

### Preparation 27

### (2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoic Acid

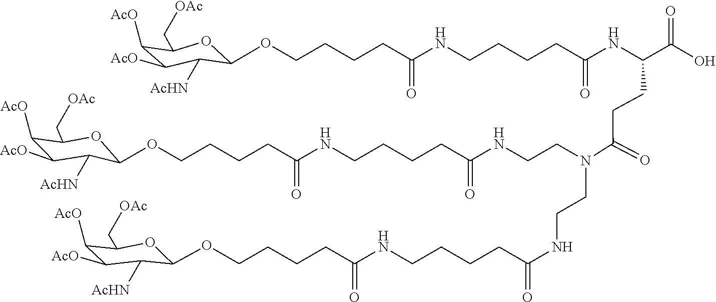 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)O)OC(COC(C)=O)C(OC(C)=O)C1OC(C)=O]

A round-bottom flask is charged with palladium on carbon (467 mg, 0.22 mmol, 5 mass %, 50% wet) and the flask is evacuated and backfilled with nitrogen three times. A solution of benzyl (2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoate (4.19 g, 2.20 mmol) in MeOH (44 mL) is added via syringe followed by three drops of acetic acid. The flask is evacuated and backfilled with 1 atm hydrogen and the mixture is stirred at ambient temperature under 1 atm hydrogen. After 2 hours, the mixture is filtered through diatomaceous earth and the filtrate is concentrated in vacuo to give the title compound (3.99 g, 99+%). ES/MS m/z 909.6 (M+2H)/2.

### Preparation 28

### Benzyl 6-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]hexanoate

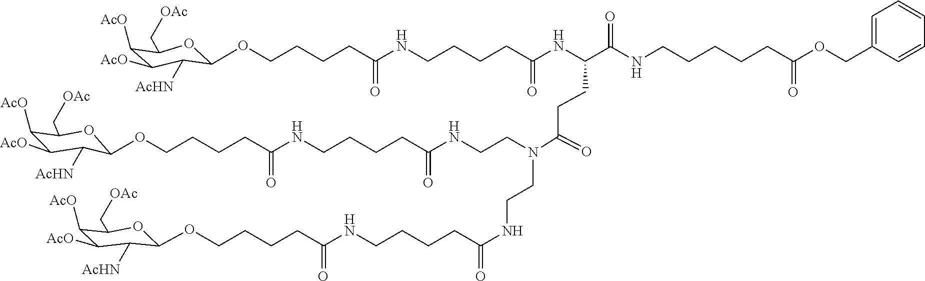 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCCCCC(=O)OCc2ccccc2)OC(COC(C)=O)C(OC(C)=O)C1OC(C)=O]

The title compound is prepared from (2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoic acid and benzyl 6-aminohexanoate hydrochloride and in a manner essentially analogous to the method of preparation 10. ES/MS m/z 1011.6 (M+2H)/2.

### Preparation 29

### 6-[[(2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]hexanoic Acid

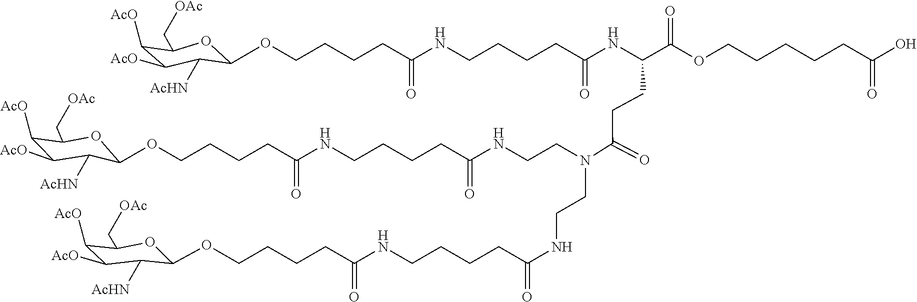 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)OCCCCCC(=O)O)OC(COC(C)=O)C(OC(C)=O)C1OC(C)=O]

A round-bottom flask is charged with palladium on carbon (24 mg, 0.01 mmol, 5% by mass, 50% wet) and the flask is evacuated and backfilled with nitrogen. A solution of benzyl 6-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]hexanoate (222 mg, 0.11 mmol) in MeOH (2.2 mL) is added via syringe followed by three drops of acetic acid. The flask is evacuated and backfilled with 1 atm hydrogen and the mixture is stirred under 1 atm hydrogen at ambient temperature. After 5 hours, the flask is purged with nitrogen and the mixture is filtered through diatomaceous earth. The filtrate is concentrated in vacuo to give the title compound (180 mg, 85%). ES/MS m/z 966.2 (M+2H)/2.

### Preparation 30

### (2,5-Dioxopyrrolidin-1-yl) 6-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]hexanoate

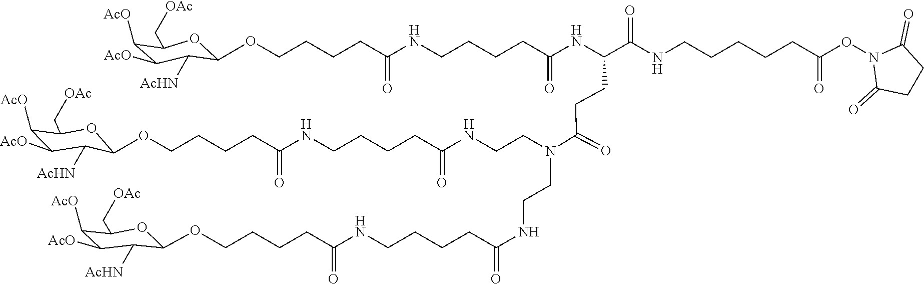 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCCCCC(=O)ON2C(=O)CCC2=O)OC(COC(C)=O)C(OC(C)=O)C1OC(C)=O]

The title compound is prepared from 6-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]hexanoic acid in a manner essentially analogous to the method of preparation 16. ES/MS m/z 1014.6 (M+2H)/2.

### Preparation 31

### Benzyl 11-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]undecanoate

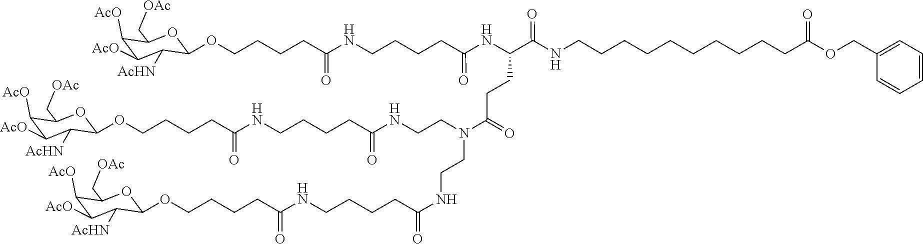 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCCCCCCCCCC(=O)OCc2ccccc2)OC(COC(C)=O)C(OC(C)=O)C1OC(C)=O]

The title compound is prepared from (2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoic acid and benzyl 11-aminoundecanoate hydrochloride in a manner essentially analogous to the method of preparation 10. ES/MS m/z 1046.6 (M+2H)/2.

### Preparation 32

### 11-[[(2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]undecanoic Acid

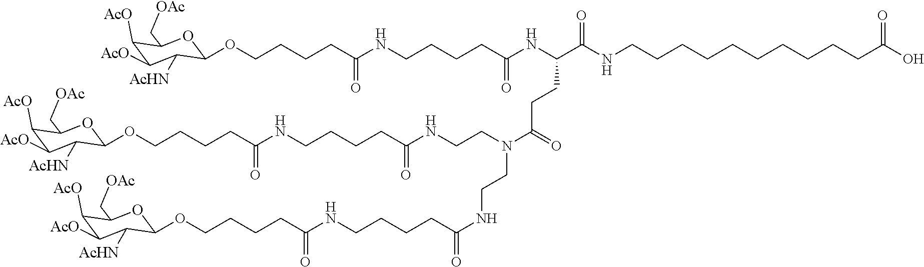 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCCCCCCCCCC(=O)O)OC(COC(C)=O)C(OC(C)=O)C1OC(C)=O]

To a round-bottom flask is added palladium on carbon (35 mg, 0.02 mmol, 5 mass %, 50% wet) and the flask is evacuated and backfilled with nitrogen three times. A solution of benzyl 11-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]undecanoate (285 mg, 80% purity, 0.11 mmol) is added via syringe. The vessel is evacuated and backfilled with 1 atm hydrogen and the mixture is then stirred at ambient temperature under 1 atm hydrogen. After stirring for 3 hours, the flask is purged with nitrogen and the mixture is filtered through diatomaceous earth. The filtrate is concentrated to give the title compound (213 mg, 79% purity, 77%). ES/MS m/z 1001.20 (M+2H)/2.

### Preparation 33

### (2,5-Dioxopyrrolidin-1-yl) 11-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]undecanoate

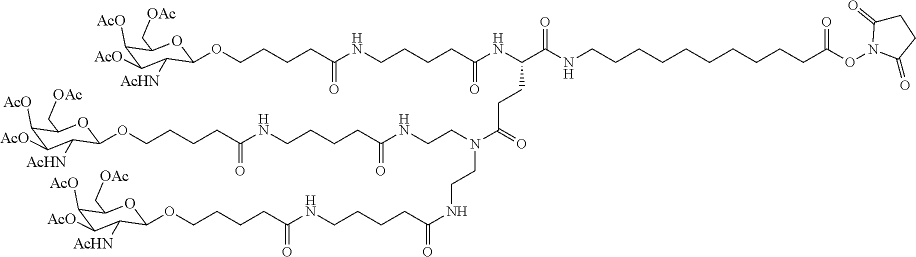 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCCCCCCCCCC(=O)ON2C(=O)CCC2=O)OC(COC(C)=O)C(OC(C)=O)C1OC(C)=O]

The title compound is prepared from 11-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]undecanoic acid in a manner essentially analogous to the method of preparation 16. ES/MS m/z 1050 (M+2H)/2

### Preparation 34

### [5-Acetamido-6-[5-[2-[[(4S)-4-[5-[3-acetamido-4,5-diacetoxy-6-(acetoxymethyl)tetrahydropyran-2-yl]oxypentanoylamino]-5-[[6-[[(1S)-1-[[bis(4-methoxyphenyl)-phenyl-methoxy]methyl]-2-[4-(hydroxymethyl)-1-piperidyl]-2-oxo-ethyl]amino]-6-oxo-hexyl]amino]-5-oxo-pentanoyl]-[2-[5-[3-acetamido-4,5-diacetoxy-6-(acetoxymethyl)tetrahydropyran-2-yl]oxypentanoylamino]ethyl]amino]ethylamino]-5-oxo-pentoxy]-3,4-diacetoxy-tetrahydropyran-2-yl]methyl Acetate

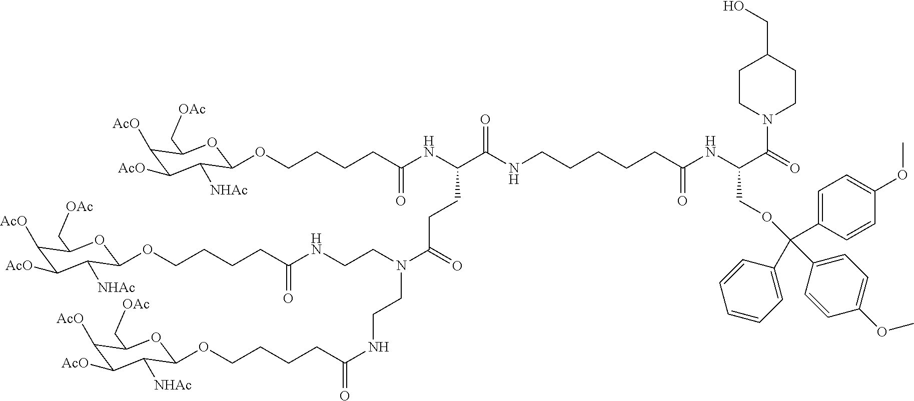 [SMILES: COc1ccc(C(OC[C@H](NC(=O)CCCCCNC(=O)[C@H](CCC(=O)N(CCNC(=O)CCCCOC2OC(COC(C)=O)C(OC(C)=O)C(OC(C)=O)C2NC(C)=O)CCNC(=O)CCCCOC2OC(COC(C)=O)C(OC(C)=O)C(OC(C)=O)C2NC(C)=O)NC(=O)CCCCOC2OC(COC(C)=O)C(OC(C)=O)C(OC(C)=O)C2NC(C)=O)C(=O)N2CCC(CO)CC2)(c2ccccc2)c2ccc(OC)cc2)cc1]

The title compound is prepared from 6-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoic acid and (2S)-2-amino-3-[bis(4-methoxyphenyl)-phenyl-methoxy]-1-[4-(hydroxymethyl)-1-piperidyl]propan-1-one in a manner essentially analogous to the method of preparation 10. ES/MS m/z 1059.2 (M−2H)/2.

### Preparation 35

### 4-[1-[[(2S)-2-[6-[[(2S)-2-[5-[3-Acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoylamino]-3-[bis(4-methoxyphenyl)-phenyl-methoxy]propanoyl]-4-piperidyl]methoxy]-4-oxo-butanoic Acid

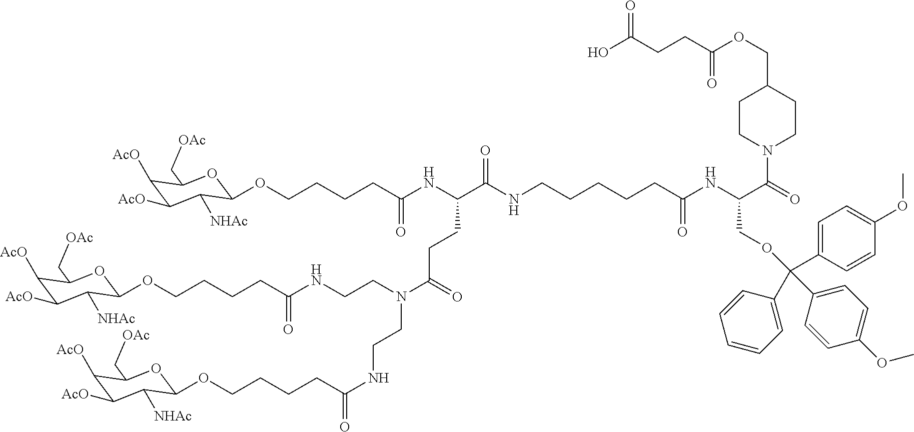 [SMILES: COc1ccc(C(OC[C@H](NC(=O)CCCCCNC(=O)[C@H](CCC(=O)N(CCNC(=O)CCCCOC2OC(COC(C)=O)C(OC(C)=O)C(OC(C)=O)C2NC(C)=O)CCNC(=O)CCCCOC2OC(COC(C)=O)C(OC(C)=O)C(OC(C)=O)C2NC(C)=O)NC(=O)CCCCOC2OC(COC(C)=O)C(OC(C)=O)C(OC(C)=O)C2NC(C)=O)C(=O)N2CCC(COC(=O)CCC(=O)O)CC2)(c2ccccc2)c2ccc(OC)cc2)cc1]

To a solution of [5-acetamido-6-[5-[2-[[(4S)-4-[5-[3-acetamido-4,5-diacetoxy-6-(acetoxymethyl)tetrahydropyran-2-yl]oxypentanoylamino]-5-[[6-[[(1S)-1-[[bis(4-methoxyphenyl)-phenyl-methoxy]methyl]-2-[4-(hydroxymethyl)-1-piperidyl]-2-oxo-ethyl]amino]-6-oxo-hexyl]amino]-5-oxo-pentanoyl]-[2-[5-[3-acetamido-4,5-diacetoxy-6-(acetoxymethyl)tetrahydropyran-2-yl]oxypentanoylamino]ethyl]amino]ethylamino]-5-oxo-pentoxy]-3,4-diacetoxy-tetrahydropyran-2-yl]methyl acetate (1.194 g, 0.56 mmol) in DCM (11 mL) is added succinic anhydride (113 mg, 1.13 mmol), TEA (0.4 mL, 3 mmol) and DMAP (213 mg, 1.69 mmol). The mixture is stirred at ambient temperature for 1 hour. After this time, the mixture is diluted with saturated NH₄Cl (200 mL) and extracted with DCM (3× 200 mL) and 3:1 CHCl₃:IPA (200 mL). The organic layers are combined, dried over sodium sulfate, filtered, and concentrated in vacuo. The resulting residue is purified by silica gel flash chromatography eluting with 0-40% MeOH/DCM and the resulting product is dried in a vacuum oven at 40° C. for 3 hours to give the title compound (1.081 g, 86%). ES/MS m/z 1109.60 (M−2H)/2.

### Preparation 36

### Resin Loading

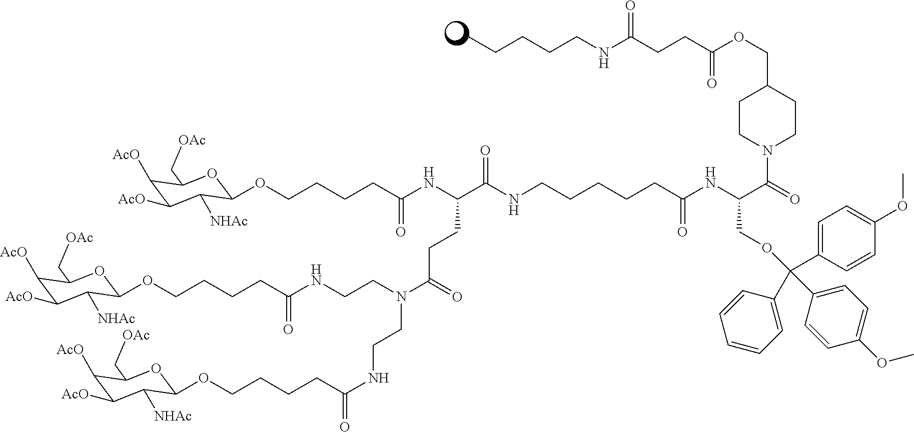 [SMILES: CCCCCNC(=O)CCC(=O)OCC1CCN(C(=O)[C@H](COC(c2ccccc2)(c2ccc(OC)cc2)c2ccc(OC)cc2)NC(=O)CCCCCNC(=O)[C@H](CCC(=O)N(CCNC(=O)CCCCOC2OC(COC(C)=O)C(OC(C)=O)C(OC(C)=O)C2NC(C)=O)CCNC(=O)CCCCOC2OC(COC(C)=O)C(OC(C)=O)C(OC(C)=O)C2NC(C)=O)NC(=O)CCCCOC2OC(COC(C)=O)C(OC(C)=O)C(OC(C)=O)C2NC(C)=O)CC1]

A solution of 4-[1-[[(2S)-2-[6-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoylamino]-3-[bis(4-methoxyphenyl)-phenyl-methoxy]propanoyl]-4-piperidyl]methoxy]-4-oxo-butanoic acid (1.00 g, 0.61 mmol) in MeCN (6 mL) and DCM (1 mL) is transferred to a resin loading cartridge. To the vessel are added 2-(1H-benzotriazol-1-yl)-1,1,3,3-tetramethyluronium hexafluorophosphate (386 mg, 0.97 mmol) and DIEA (0.25 mL, 0.48 mmol) and the cartridge is shaken at ambient temperature for 5 minutes. After this time, 1000 Å LCAA controlled-pore glass resin (5.39 g, 90 μmol/g loading, purchased from ChemGenes) is added and the mixture is shaken at ambient temperature for 18 hours. After this time, the cartridge is drained by suction and the resin is washed by shaking with DCM (10 mL) for 10 minutes. The cartridge is drained and the washing and draining procedure is repeated with 10% MeOH/DCM (10 mL) and Et₂O (10 mL). After draining, a solution of acetic anhydride (6.4 mL), pyridine (20 mL) and TEA (0.22 mL) is added and the cartridge is shaken for 2 hours. After this time, the cartridge is drained and the washing and draining procedure above is repeated using DCM (10 mL), 10% MeOH/DCM (10 mL) and diethyl ether (10 mL). After draining, the resin is dried under vacuum for 30 minutes. The resin loading is determined using a standard trityl assay. The resin loading was calculated to be 34.7 μmol/g.

### Preparation 37

### Benzyl 2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]acetate

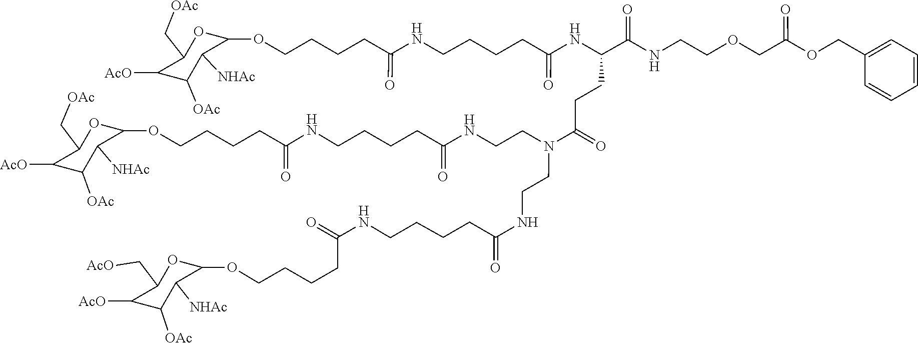 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCOCC(=O)OCc2ccccc2)OC(COC(C)=O)C(OC(C)=O)C1OC(C)=O]

The title compound is prepared from (2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoic acid and benzyl 2-(2-aminoethoxy)acetate hydrochloride in a manner essentially analogous to the method of preparation 10. ES/MS m/z 1005.2 (M+2H/2).

### Preparation 38

### 2-[2-[[(2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]acetic Acid

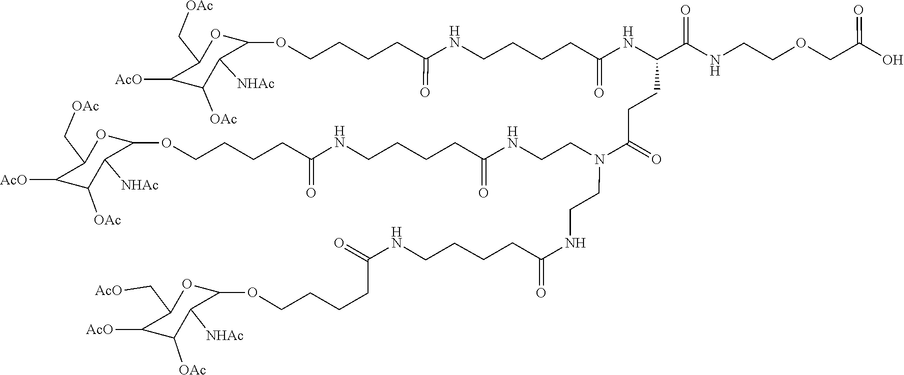 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCOCC(=O)O)OC(COC(C)=O)C(OC(C)=O)C1OC(C)=O]

Benzyl 2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]acetate (0.120 mmol, 240 mg) is combined with 5% Pd/C (1.17 mmol, 124 mg) in MeOH (12.0 ml). The mixture is hydrogenated on a Parr shaker (ambient temperature, 10 psi) for 48 minutes, filtered through diatomaceous earth, and concentrated in vacuo to give the title compound as a gray solid (187 mg, 82%). ES/MS m/z 960.0 (M+2H/2).

### Preparation 39

### (2,3,5,6-Tetrafluorophenyl) 2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]acetate

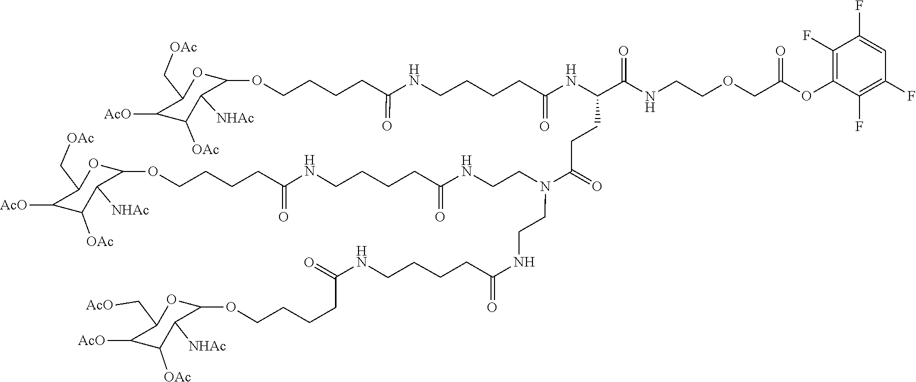 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCOCC(=O)Oc2c(F)c(F)cc(F)c2F)OC(COC(C)=O)C(OC(C)=O)C1OC(C)=O]

To 2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]acetic acid (0.096 mmol, 184 mg) and DIEA (0.765 mmol, 140 μL) in DCM (3.0 ml) is added (2,3,5,6-tetrafluorophenyl) 2,2,2-trifluoroacetate (0.383 mmol, 100 mg) to the mixture dropwise. The mixture is stirred at ambient temperature for 16 hours. The reaction mixture is purified directly by silica gel flash chromatography eluting with 0% to 50% MeOH/DCM to give the title compound as a tan solid (197 mg, 99%). ES/MS m/z 1034.0 (M+2H/2).

### Preparation 40

### Benzyl 2-[2-[2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]ethoxy]ethoxy]acetate

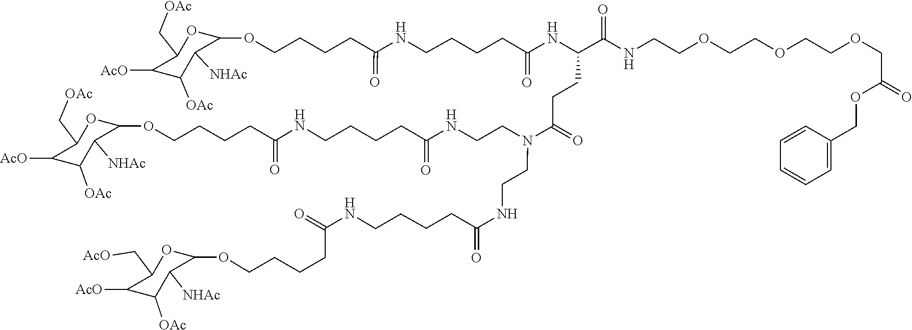 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCOCCOCCOCC(=O)OCc2ccccc2)OC(COC(C)=O)C(OC(C)=O)C1OC(C)=O]

The title compound is prepared from (2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoic acid and benzyl 2-[2-[2-(2-aminoethoxy) ethoxy]ethoxy]acetate hydrochloride in a manner essentially analogous to the method of preparation 10. ES/MS m/z 1049.0 (M+2H/2).

### Preparation 41

### 2-[2-[2-[2-[[(2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]ethoxy]ethoxy]acetic Acid

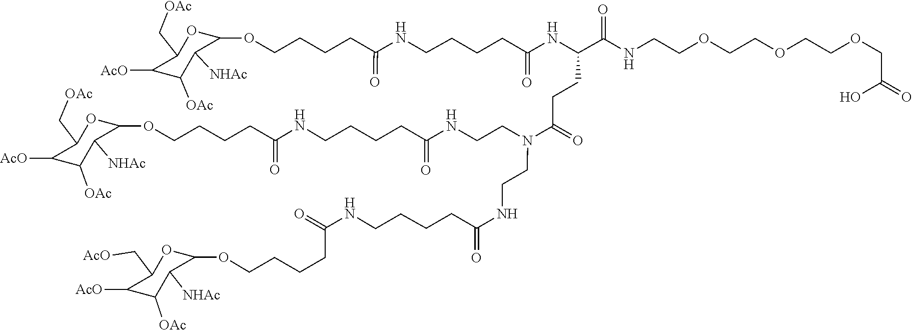 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCOCCOCCOCC(=O)O)OC(COC(C)=O)C(OC(C)=O)C1OC(C)=O]

Benzyl 2-[2-[2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]ethoxy]ethoxy]acetate (0.118 mmol, 247 mg) is combined with 5% Pd/C (1.17 mmol, 124 mg) in MeOH (12.0 mL). The mixture is hydrogenated on a Parr shaker (ambient temperature, 10 psi) for 1 hour, filtered through diatomaceous earth, and concentrated in vacuo to give the title compound as a gray solid (227 mg, 96%). ES/MS m/z 1004.0 (M+2H/2).

### Preparation 42

### (2,3,5,6-Tetrafluorophenyl) 2-[2-[2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]ethoxy]ethoxy]acetate

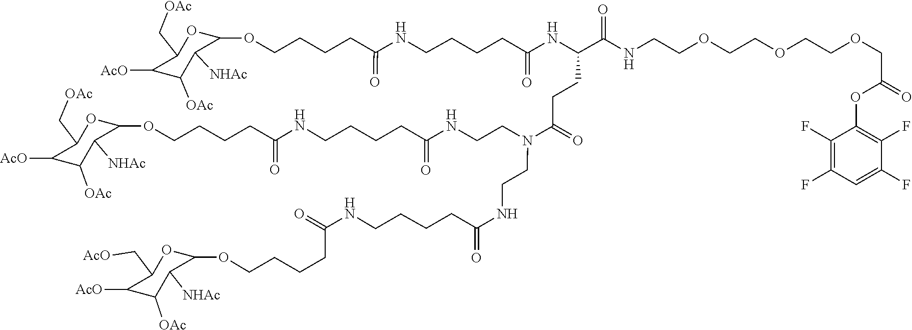 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCOCCOCCOCC(=O)Oc2c(F)c(F)cc(F)c2F)OC(COC(C)=O)C(OC(C)=O)C1OC(C)=O]

To 2-[2-[2-[2-[[(2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]ethoxy]ethoxy]acetic acid (0.111 mmol, 222 mg) and DIEA (0.883 mmol, 154 μL) in DCM (3.0 ml) is added (2,3,5,6-tetrafluorophenyl) 2,2,2-trifluoroacetate (0.443 mmol, 116 mg) to the mixture dropwise. The mixture is stirred at ambient temperature for 16 hours. The reaction mixture is purified directly by silica gel flash chromatography eluting with 0% to 50% MeOH/DCM to give the title compound as a tan solid (174 mg, 73%). ES/MS m/z 1078.2 (M+2H/2).

### Example 1: Conjugation Protocol

For the synthesis of GalNAc-conjugated sense strands, a sense strand with a 3′ C6-NH₂ functional group was first synthesized using standard phosphoramidite chemistry. A stock solution of GalNAc ligand-NHS ester (10 mmol/L in acetonitrile; 1 eq) was prepared. Borate buffer (10% v/v; 20×) was added to oligonucleotide C6-NH₂ sense strand in an Eppendorf tube, then GalNAc ligand (5 eq) was added. The mixture was shaken at ambient temperature for 16 hours. After this time, the mixture was transferred to a 15 mL falcon tube, ammonium hydroxide (28 mass %) was added, and the mixture was shaken at ambient temperature for 2 hours. The ammonia was then removed in vacuo. The residue was purified by ion-exchange chromatography. Conditions: Solvent A: 15% MeCN/20 mM NaH₂PO₄, Solvent B: 15% MeCN/20 mM NaH₂PO₄, 1M NaBr; 35-55% B over 5 CV at 8 mL/min, column temperature 60° C. The desired fractions were pooled and desalted by spin-filtration using an Eppendorf centrifuge or desalting column. After desalting, the material was recovered and OD and volume were measured to obtain concentration.

Alternatively, conjugation to the 5′ position of the sense strand through immobilizing the GalNAc ligand on microporous polystyrene resin or controlled pore glass and synthesized using established solid phase oligonucleotide synthesis methods with 5′-CE ß-cyanoethyl) phosphoramidites.

Alternatively, the GalNAc ligand was converted to a suitable phosphoramidite and delivered to the 5′ position of the sense strand using standard phosphoramidite chemistry.

### Example 2: Annealing

To generate the siRNA duplexes of a sense and antisense strand, the following procedures were performed. To a falcon tube containing oligonucleotide sense strand-GalNAc conjugate, the corresponding antisense oligonucleotide (1 eq) was added and vortexed for 10 seconds before spin-filtering through 100K MWCO Amicon filter unit to remove particulates. The filtrate was recovered and concentrated in vacuo on a Genevac evaporator. The residue was reconstituted in 1×PBS, filtered through 0.2μ filter, and OD and volume were measured to obtain concentration.

An endotoxin test was performed using a Limulus amebocyte lysate on an Endosafe®-nexgen PTS instrument.

TABLE 1
Exemplary molecules synthesized utilizing the aforementioned conjugation and annealing protocols.

| Molecule | |
|---|---|
| Identifier | Ligand attached to 3′ of sense strand |
| 1 | 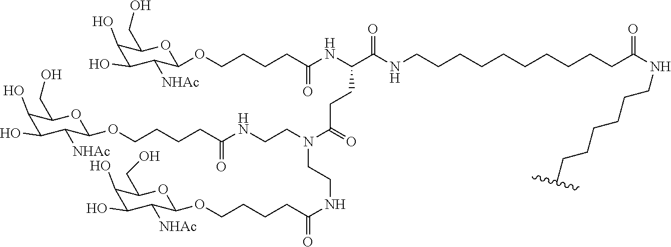 [SMILES: *CCCCCCNC(=O)CCCCCCCCCCNC(=O)[C@H](CCC(=O)N(CCNC(=O)CCCCOC1OC(CO)C(O)C(O)C1NC(C)=O)CCNC(=O)CCCCOC1OC(CO)C(O)C(O)C1NC(C)=O)NC(=O)CCCCOC1OC(CO)C(O)C(O)C1NC(C)=O] |
| 2 | 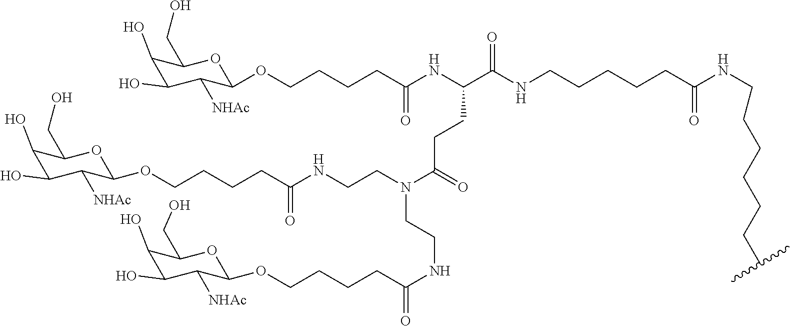 [SMILES: *CCCCCCNC(=O)CCCCCNC(=O)[C@H](CCC(=O)N(CCNC(=O)CCCCOC1OC(CO)C(O)C(O)C1NC(C)=O)CCNC(=O)CCCCOC1OC(CO)C(O)C(O)C1NC(C)=O)NC(=O)CCCCOC1OC(CO)C(O)C(O)C1NC(C)=O] |
| 3 | 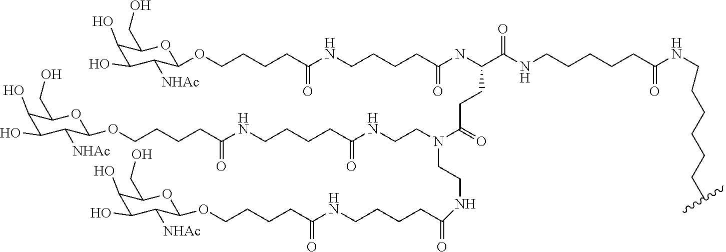 [SMILES: *CCCCCCNC(=O)CCCCCNC(=O)[C@H](CCC(=O)N(CCNC(=O)CCCCNC(=O)CCCCOC1OC(CO)C(O)C(O)C1NC(C)=O)CCNC(=O)CCCCNC(=O)CCCCOC1OC(CO)C(O)C(O)C1NC(C)=O)NC(=O)CCCCNC(=O)CCCCOC1OC(CO)C(O)C(O)C1NC(C)=O] |
| 4 | 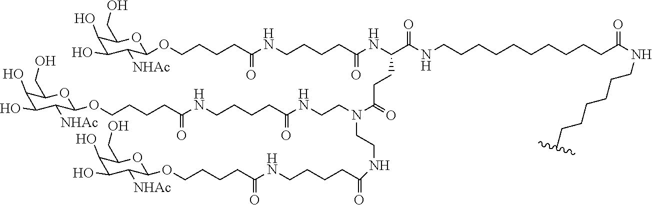 [SMILES: *CCCCCCNC(=O)CCCCCCCCCCNC(=O)[C@H](CCC(=O)N(CCNC(=O)CCCCNC(=O)CCCCOC1OC(CO)C(O)C(O)C1NC(C)=O)CCNC(=O)CCCCNC(=O)CCCCOC1OC(CO)C(O)C(O)C1NC(C)=O)NC(=O)CCCCNC(=O)CCCCOC1OC(CO)C(O)C(O)C1NC(C)=O] |
| 5 | 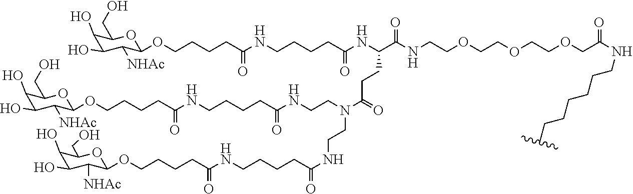 [SMILES: *CCCCCCNC(=O)COCCOCCOCCNC(=O)[C@H](CCC(=O)N(CCNC(=O)CCCCNC(=O)CCCCOC1OC(CO)C(O)C(O)C1NC(C)=O)CCNC(=O)CCCCNC(=O)CCCCOC1OC(CO)C(O)C(O)C1NC(C)=O)NC(=O)CCCCNC(=O)CCCCOC1OC(CO)C(O)C(O)C1NC(C)=O] |
| 6 | 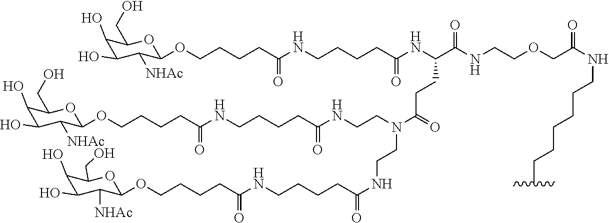 [SMILES: *CCCCCCNC(=O)COCCNC(=O)[C@H](CCC(=O)N(CCNC(=O)CCCCNC(=O)CCCCOC1OC(CO)C(O)C(O)C1NC(C)=O)CCNC(=O)CCCCNC(=O)CCCCOC1OC(CO)C(O)C(O)C1NC(C)=O)NC(=O)CCCCNC(=O)CCCCOC1OC(CO)C(O)C(O)C1NC(C)=O] |

### Example 3

### General Procedure for Oligo Synthesis Using GalNAc-Functionalized CPG

Oligo synthesis was conducted on a MerMade™ 12 instrument using phosphoramidite chemistry. Sense strands were synthesized from the prefunctionalized GalNAc solid support and antisense strands were synthesized using standard support preloaded with the first nucleotide of the oligo sequence. Oligos were cleaved and deprotected using concentrated ammonium hydroxide solution (28% by mass) and purified by ion exchange chromatography using conditions described above. Desalting, annealing, and endotoxin testing were conducted.

The sequence of antisense oligonucleotides were designed using 15 to 50 nucleotides of the antisense strands described herein, including those in Table 2A, 2B and Table 3A, 3B.

Exemplary antisense strand sequences of 23 nucleotides in length are shown in Table 2A and 2B below, which may be optionally further modified and synthesized and incorporated into the RNAi agents, as described herein.

TABLE 2A
Modified ANGPTL8 Sequences for GalNAc-RNAi Agents

| Duplex | | | Sequence (5′ to 3′ for each sense strand |
|---|---|---|---|
| NO: | SEQ ID NO: | | (SS) and antisense strand (AS)) |
| D:1 | SEQ ID NO: 1 | SS: | mA*mG*mCmAmGmCmAmUfCfGfGmCmUmGmCmGmAmCmA*mG*mA |
| | SEQ ID NO: 6 | AS: | [Phos]mU*fC*mUmGfUmCfGmCmAmGmCmCmGfAmUfGmCmUmGmCmU*mG*mU |
| D:2 | SEQ ID NO: 2 | SS: | mG*mG*mAmGmCmUmGmAfCfCfCmUmGmCmUmCmUmUmC*mC*mA |
| | SEQ ID NO: 7 | AS: | [Phos]mU*fG*mGmAfAmGfAmGmCmAmGmGmGfUmCfAmGmCmUmCmC*mU*mC |
| D:3 | SEQ ID NO: 3 | SS: | mG*mA*mCmUmCmAmGmAfUfGfGmAmGmGmAmGmGmAmU*mA*mU |
| | SEQ ID NO: 8 | AS: | [Phos]mA*fU*mAmUfCmCfUmCmCmUmCmCmAfUmCfUmGmAmGmUmC*mU*mC |
| D:4 | SEQ ID NO: 4 | SS: | mC*mU*mUmAmAmAmGmGfCfUfCmAmCmGmCmUmGmAmC*mA*mA |
| | SEQ ID NO: 9 | AS: | [Phos]mU*fU*mGmUfCmAfGmCmGmUmGmAmGfCmCfUmUmUmAmAmG*mA*mC |
| D:5 | SEQ ID NO: 5 | SS: | mA*mA*mGmCmCmUmGmUfUfGfGmAmGmAmCmUmCmAmG*mA*mU |
| | SEQ ID NO: 10 | AS: | [Phos]mA*fU*mCmUfGmAfGmUmCmUmCmCmAfAmCfAmGmGmCmUmU*mG*mC |

TABLE 2B
Modified ANGPTL8 Sequences for GalNAc-RNAi Agents

| Duplex | | Sequence (5′ to 3′ for each sense strand |
|---|---|---|
| NO: | SEQ ID NO: | (SS) and antisense strand (AS)) |
| D:104 | SEQ ID NO: 1 | mA*mG*mCmAmGmCmAmUfCfGfGmCmUmGmCmGmAmCmA*mG*mA |
| | SEQ ID NO: 11 | mU*fC*mUmGfUmCfGmCmAmGmCmCmGfAmUfGmCmUmGmCmU*mG*mU |
| D:105 | SEQ ID NO: 2 | mG*mG*mAmGmCmUmGmAfCfCfCmUmGmCmUmCmUmUmC*mC*mA |
| | SEQ ID NO: 12 | mU*fG*mGmAfAmGfAmGmCmAmGmGmGfUmCfAmGmCmUmCmC*mU*mC |
| D:106 | SEQ ID NO: 3 | mG*mA*mCmUmCmAmGmAfUfGfGmAmGmGmAmGmGmAmU*mA*mU |
| | SEQ ID NO: 13 | mA*fU*mAmUfCmCfUmCmCmUmCmCmAfUmCfUmGmAmGmUmC*mU*mC |
| D:107 | SEQ ID NO: 4 | mC*mU*mUmAmAmAmGmGfCfUfCmAmCmGmCmUmGmAmC*mA*mA |
| | SEQ ID NO: 14 | mU*fU*mGmUfCmAfGmCmGmUmGmAmGfCmCfUmUmUmAmAmG*mA*mC |
| D:108 | SEQ ID NO: 5 | mA*mA*mGmCmCmUmGmUfUfGfGmAmGmAmCmUmCmAmG*mA*mU |
| | SEQ ID NO: 15 | mA*fU*mCmUfGmAfGmUmCmUmCmCmAfAmCfAmGmGmCmUmU*mG*mC |

TABLE 3A
Modified sense and antisense strands for the GalNAc-ANGPTL8

| RNAi | | |
|---|---|---|
| Agents | | |
| herein | | Sequence (5′ to 3′ for each sense strand (SS) |
| Duplex NO: | SEQ ID NO: | and antisense strand (AS)) |
| D:6 | SEQ ID NO: 16 | mU*mC*mGmGmGmCmAmAfGfCfCmUmGmUmUmGmGmAmG*mA*mA |
| | SEQ ID NO: 113 | Phos mU*fU*mCmUfCmCfAmAmCmAmGmGmCfUmUfGmCmCmCmGmA*mA*mG |
| D:7 | SEQ ID NO: 17 | mA*mA*mUmCmUmGmCmCfUfGfGmAmUmGmGmAmAmCmU*mG*mA |
| | SEQ ID NO: 114 | Phos mU*fC*mAmGfUmUfCmCmAmUmCmCmAfGmGfCmAmGmAmUmU*mC*mA |
| D:8 | SEQ ID NO: 18 | mC*mC*mUmGmGmAmUmGfGfAfAmCmUmGmAmGmGmAmC*mC*mA |
| | SEQ ID NO: 115 | Phos mU*fG*mGmUfCmCfUmCmAmGmUmUmCfCmAfUmCmCmAmGmG*mC*mA |
| D:9 | SEQ ID NO: 19 | mU*mU*mAmAmAmGmCmAfGfAfGmUmCmGmUmGmGmCmA*mU*mA |
| | SEQ ID NO: 116 | Phos mU*fA*mUmGfCmCfAmCmGmAmCmUmCfUmGfCmUmUmUmAmA*mU*mG |
| D:10 | SEQ ID NO: 20 | mU*mG*mCmGmAmCmAmGfAfUfCmCmAmGmGmAmGmAmG*mA*mA |
| | SEQ ID NO: 117 | Phos mU*fU*mCmUfCmUfCmCmUmGmGmAmUfCmUfGmUmCmGmCmA*mG*mC |
| D:11 | SEQ ID NO: 21 | mU*mG*mAmAmUmCmUmGfCfCfUmGmGmAmUmGmGmAmA*mC*mA |
| | SEQ ID NO: 118 | Phos mU*fG*mUmUfCmCfAmUmCmCmAmGmGfCmAfGmAmUmUmCmA*mG*mG |
| D:12 | SEQ ID NO: 22 | mG*mA*mGmCmUmGmCmCfUfGfUmUmCmAmCmUmGmGmG*mA*mA |
| | SEQ ID NO: 119 | Phos mU*fU*mCmCfCmAfGmUmGmAmAmCmAfGmGfCmAmGmCmUmC*mC*mU |
| D:13 | SEQ ID NO: 23 | mA*mG*mGmUmCmUmUmAfAfAfGmGmCmUmCmAmCmGmC*mU*mA |
| | SEQ ID NO: 120 | Phos mU*fAmGmCfGmUfGmAmGmCmCmUmUfUmAfAmGmAmCmCmU*mC*mA |
| D:14 | SEQ ID NO: 24 | mU*mC*mAmCmUmGmGmGfAfUfCmAmGmCmCmAmGmGmG*mC*mA |
| | SEQ ID NO: 121 | Phos mU*fG*mCmCfCmUfGmGmCmUmGmAmUfCmCfCmAmGmUmGmA*mA*mC |
| D:15 | SEQ ID NO: 25 | mU*mC*mAmUmUmAmAmAfGfCfAmGmAmGmUmCmGmUmG*mG*mA |
| | SEQ ID NO: 122 | Phos mU*fC*mCmAfCmGfAmCmUmCmUmGmCfUmUfUmAmAmUmGmA*mG*mG |
| D:16 | SEQ ID NO: 26 | mG*mA*mAmCmAmGmCmCfUfGfGmGmUmCmUmCmUmAmU*mG*mA |
| | SEQ ID NO: 123 | Phos mU*fC*mAmUfAmGfAmGmAmCmCmCmAfGmGfCmUmGmUmUmC*mC*mU |
| D:17 | SEQ ID NO: 27 | mG*mG*mGmGmCmAmGmGfAfGfGmUmCmAmGmCmCmGmG*mG*mA |
| | SEQ ID NO: 124 | Phos mU*fC*mCmCfGmGfCmUmGmAmCmCmUfCmCfUmGmCmCmCmC*mA*mG |
| D:18 | SEQ ID NO: 28 | mA*mG*mCmGmGmCmUmAfGfAfAmGmUmCmCmAmGmCmU*mG*mA |
| | SEQ ID NO: 125 | Phos mU*fC*mAmGfCmUfGmGmAmCmUmUmCfUmAfGmCmCmGmCmU*mG*mC |
| D:19 | SEQ ID NO: 29 | mG*mA*mGmAmUmGmGmUfGfGfCmAmCmAmGmCmAmGmC*mA*mA |
| | SEQ ID NO: 126 | Phos mU*fU*mGmCfUmGfCmUmGmUmGmCmCfAmCfCmAmUmCmUmC*mC*mC |
| D:20 | SEQ ID NO: 30 | mG*mG*mCmUmGmAmCmAfAfAfGmGmCmCmAmGmGmAmA*mC*mA |
| | SEQ ID NO: 127 | Phos mU*fG*mUmUfCmCfUmGmGmCmCmUmUfUmGfUmCmAmGmCmC*mG*mU |
| D:21 | SEQ ID NO: 31 | mG*mU*mCmCmAmGmCmUfGfAfGmGmAmGmCmGmCmCmU*mG*mA |
| | SEQ ID NO: 128 | Phos mU*fC*mAmGfGmCfGmCmUmCmCmUmCfAmGfCmUmGmGmAmC*mU*mU |
| D:22 | SEQ ID NO: 32 | mC*mA*mUmUmAmAmAmGfCfAfGmAmGmUmCmGmUmGmG*mC*mA |
| | SEQ ID NO: 129 | Phos mU*fG*mCmCfAmCfGmAmCmUmCmUmGfCmUfUmUmAmAmUmG*mA*mG |
| D:23 | SEQ ID NO: 33 | mU*mC*mCmCmAmGmCmCfUfGfAmAmUmCmUmGmCmCmU*mG*mA |
| | SEQ ID NO: 130 | Phos mU*fC*mAmGfGmCfAmGmAmUmUmCmAfGmGfCmUmGmGmGmA*mG*mC |
| D:24 | SEQ ID NO: 34 | mG*mG*mAmUmGmCmAmGfCfCfCmAmGmGmAmAmCmUmU*mC*mA |
| | SEQ ID NO: 131 | Phos mU*fG*mAmAfGmUfUmCmCmUmGmGmGfCmUfGmCmAmUmCmC*mC*mG |
| D:25 | SEQ ID NO: 35 | mG*mC*mUmAmGmAmAmGfUfCfCmAmGmCmUmGmAmGmG*mA*mA |
| | SEQ ID NO: 132 | Phos mU*fU*mCmCfUmCfAmGmCmUmGmGmAfCmUfUmCmUmAmGmC*mC*mG |
| D:26 | SEQ ID NO: 36 | mC*mA*mCmAmGmCmAmGfCfAfUmCmGmGmCmUmGmCmG*mA*mA |
| | SEQ ID NO: 133 | Phos mU*fU*mCmGfCmAfGmCmCmGmAmUmGfCmUfGmCmUmGmUmG*mC*mC |
| D:27 | SEQ ID NO: 37 | mG*mG*mAmAmCmUmUmCfGfGfGmCmAmAmGmCmCmUmG*mU*mA |
| | SEQ ID NO: 134 | Phos mU*fA*mCmAfGmGfCmUmUmGmCmCmCfGmAfAmGmUmUmCmC*mU*mG |
| D:28 | SEQ ID NO: 38 | mA*mC*mUmGmGmGmAmUfCfAfGmCmCmAmGmGmGmCmG*mC*mA |
| | SEQ ID NO: 135 | Phos mU*fG*mCmGfCmCfCmUmGmGmCmUmGfAmUfCmCmCmAmGmU*mG*mA |
| D:29 | SEQ ID NO: 39 | mA*mG*mAmAmGmUmCmCfAfGfCmUmGmAmGmGmAmGmC*mG*mA |
| | SEQ ID NO: 136 | Phos mU*fC*mGmCfUmCfCmUmCmAmGmCmUfGmGfAmCmUmUmCmU*mA*mG |
| D:30 | SEQ ID NO: 40 | mG*mC*mAmUmCmGmGmCfUffCmGmAmCmAmGmAmUmC*mC*mA |
| | SEQ ID NO: 137 | Phos mU*fG*mGmAfUmCfUmGmUmCmGmCmAfGmCfCmGmAmUmGmC*mU*mG |
| D:31 | SEQ ID NO: 41 | mA*mG*mAmUmGmGmUmGfGfCfAmCmAmGmCmAmGmCmA*mU*mA |
| | SEQ ID NO: 138 | Phos mU*fA*mUmGfCmUfGmCmUmGmUmGmCfCmAfCmCmAmUmCmU*mC*mC |
| D:32 | SEQ ID NO: 42 | mG*mG*mUmGmGmCmAmCfAfGfCmAmGmCmAmUmCmGmG*mC*mA |
| | SEQ ID NO: 139 | Phos mU*fG*mCmCfGmAfUmGmCmUmGmCmUfGmUfGmCmCmAmCmC*mA*mU |
| D:33 | SEQ ID NO: 43 | mA*mC*mAmGmCmAmGmCfAfUfCmGmGmCmUmGmCmGmA*mC*mA |
| | SEQ ID NO: 140 | Phos mU*fG*mUmCfGmCfAmGmCmCmGmAmUfGmCfUmGmCmUmGmU*mG*mC |
| D:34 | SEQ ID NO: 44 | mA*mA*mGmGmCmUmCmAfCfGfCmUmGmAmCmAmAmGmC*mA*mA |
| | SEQ ID NO: 141 | Phos mU*fU*mGmCfUmUfGmUmCmAmGmCmGfUmGfAmGmCmCmUmU*mU*mA |
| D:35 | SEQ ID NO: 45 | mA*mG*mCmCmUmGmAmAfUfCfUmGmCmCmUmGmGmAmU*mG*mA |
| | SEQ ID NO: 142 | Phos mU*fC*mAmUfCmCfAmGmGmCmAmGmAfUmUfCmAmGmGmCmU*mG*mG |
| D:36 | SEQ ID NO: 46 | mA*mU*mCmGmGmCmUmGfCfGfAmCmAmGmAmUmCmCmA*mG*mA |
| | SEQ ID NO: 143 | Phos mU*fC*mUmGfGmAfUmCmUmGmUmCmGfCmAfGmCmCmGmAmU*mG*mC |
| D:37 | SEQ ID NO: 47 | mA*mG*mUmCmCmAmGmCfUfGfAmGmGmAmGmCmGmCmC*mU*mA |
| | SEQ ID NO: 144 | Phos mU*fA*mGmGfCmGfCmUmCmCmUmCmAfGmCfUmGmGmAmCmU*mU*mC |
| D:38 | SEQ ID NO: 48 | mC*mG*mGmCmAmGmAmGfGfCfGmGmGmAmGmAmUmGmG*mU*mA |
| | SEQ ID NO: 145 | Phos mU*fA*mCmCfAmUfCmUmCmCmCmGmCfCmUfCmUmGmCmCmG*mC*mU |
| D:39 | SEQ ID NO: 49 | mA*mU*mGmCmUmGmCmAfAfGfGmAmAmCmAmCmUmUmC*mC*mA |
| | SEQ ID NO: 146 | Phos mU*fG*mGmAfAmGfUmGmUmUmCmCmUfUmGfCmAmGmCmAmU*mG*mA |
| D:40 | SEQ ID NO: 50 | mC*mU*mUmCmGmGmGmCfAfAfGmCmCmUmGmUmUmGmG*mA*mA |
| | SEQ ID NO: 147 | Phos mU*fU*mCmCfAmAfCmAmGmGmCmUmUfGmCfCmCmGmAmAmG*mU*mU |
| D:41 | SEQ ID NO: 51 | mG*mG*mAmGmCmUmGmCfCfUfGmUmUmCmAmCmUmGmG*mG*mA |
| | SEQ ID NO: 148 | Phos mU*fC*mCmCfAmGfUmGmAmAmCmAmGfGmCfAmGmCmUmCmC*mU*mC |
| D:42 | SEQ ID NO: 52 | mG*mC*mCmUmGmGmAmUfGfGfAmAmCmUmGmAmGmGmA*mC*mA |
| | SEQ ID NO: 149 | Phos mU*fG*mUmCfCmUfCmAmGmUmUmCmCfAmUfCmCmAmGmGmC*mA*mG |
| D:43 | SEQ ID NO: 53 | mC*mA*mAmGmGmAmAmCfAfCfUmUmCmCmAmCmGmCmC*mC*mA |
| | SEQ ID NO: 150 | Phos mU*fG*mGmGfCmGfUmGmGmAmAmGmUfGmUfUmCmCmUmUmG*mC*mA |
| D:45 | SEQ ID NO: 54 | mG*mG*mCmUmAmGmAmAfGfUfCmCmAmGmCmUmGmAmG*mG*mA |
| | SEQ ID NO: 151 | Phos mU*fC*mCmUfCmAfGmCmUmGmGmAmCfUmUfCmUmAmGmCmC*mG*mC |
| D:46 | SEQ ID NO: 55 | mU*mG*mAmGmGmUmCmUfUfAfAmAmGmGmCmUmCmAmC*mG*mA |
| | SEQ ID NO: 152 | Phos mU*fC*mGmUfGmAfGmCmCmUmUmUmAfAmGfAmCmCmUmCmA*mA*mA |
| D:47 | SEQ ID NO: 56 | mG*mG*mGmCmAmAmGmCfCfUfGmUmUmGmGmAmGmAmC*mU*mA |
| | SEQ ID NO: 153 | Phos mU*fA*mGmUfCmUfCmCmAmAmCmAmGfGmCfUmUmGmCmCmC*mG*mA |
| D:48 | SEQ ID NO: 57 | mG*mC*mGmGmCmUmAmGfAfAfGmUmCmCmAmGmCmUmG*mA*mA |
| | SEQ ID NO: 154 | Phos mU*fU*mCmAfGmCfUmGmGmAmCmUmUfCmUfAmGmCmCmGmC*mU*mG |
| D:49 | SEQ ID NO: 58 | mU*mG*mGmCmAmCmAmGfCfAfGmCmAmUmCmGmGmCmU*mG*mA |
| | SEQ ID NO: 155 | Phos mU*fC*mAmGfCmCfGmAmUmGmCmUmGfCmUfGmUmGmCmCmA*mC*mC |
| D:50 | SEQ ID NO: 59 | mA*mU*mGmGmUmGmGmCfAfCfAmGmCmAmGmCmAmUmC*mG*mA |
| | SEQ ID NO: 156 | Phos mU*fC*mGmAfUmGfCmUmGmCmUmGmUfGmCfCmAmCmCmAmU*m*mU |
| D:51 | SEQ ID NO: 60 | mA*mG*mAmAmUmUmUmGfAfGfGmUmCmUmUmAmAmAmG*mG*mA |
| | SEQ ID NO: 157 | Phos mU*fC*mCmUfUmUfAmAmGmAmCmCmUfCmAfAmAmUmUmCmU*mC*mG |
| D:52 | SEQ ID NO: 61 | mG*mG*mAmAmCmAmGmCfCfUfGmGmGmUmCmUmCmUmA*mU*mA |
| | SEQ ID NO: 158 | Phos mU*fA*mUmAfGmAfGmAmCmCmCmAmGfGmCfUmGmUmUmCmC*mU*mG |
| D:53 | SEQ ID NO: 62 | mA+mG*mGmCmUmCmAmCfGfCfUmGmAmCmAmAmGmCmA*mG*mA |
| | SEQ ID NO: 159 | Phos mU*fC*mUmGfCmUfUmGmUmCmAmGmCfGmUfGmAmGmCmCmU*mU*mU |
| D:54 | SEQ ID NO: 63 | mG*mG*mCmAmCmAmGmCfAfGfCmAmUmCmGmGmCmUmG*mC*mA |
| | SEQ ID NO: 160 | Phos mU*fG*mCmAfGmCfCmGmAmUmGmCmUfGmCfUmGmUmGmCmC*mA*mC |
| D:55 | SEQ ID NO: 64 | mC*mG*mGmGmCmAmAmGfCfCfUmGmUmUmGmGmAmGmA*mC*mA |
| | SEQ ID NO: 161 | Phos mU*fG*mUmCfUmCfCmAmAmCmAmGmGfCmUfUmGmCmCmCmG*mA*mA |
| D:56 | SEQ ID NO: 65 | mA*mA*mAmGmGmCmUmCfAfCfGmCmUmGmAmCmAmAmG*mC*mA |
| | SEQ ID NO: 162 | Phos mU*fG*mCmUfUmGfUmCmAmGmCmGmUfGmAfGmCmCmUmUmU*mA*mA |
| D:57 | SEQ ID NO: 66 | mG*mA*mAmUmCmUmGmCfCfUfGmGmAmUmGmGmAmAmC*mU*mA |
| | SEQ ID NO: 163 | Phos mU*fA*mGmUfUmCfCmAmUmCmCmAmGfGmCfAmGmAmUmUmC*mA*mG |
| D:58 | SEQ ID NO: 67 | mC*mC*mUmGmUmUmCmAfCfUfGmGmGmAmUmCmAmGmC*mC*mA |
| | SEQ ID NO: 164 | Phos mU*fG*mGmCfUmGfAmUmCmCmCmAmGfUmGfAmAmCmAmGmG*mC*mA |
| D:59 | SEQ ID NO: 68 | mA*mU*mUmAmAmAmGmCfAfGfAmGmUmCmGmUmGmGmC*mA*mA |
| | SEQ ID NO: 165 | Phos mU*fU*mGmCfCmAfCmGmAmCmUmCmUfGmCfUmUmUmAmAmU*mG*mA |
| D:60 | SEQ ID NO: 69 | mU*mG*mGmUmGmGmCmAfCfAfGmCmAmGmCmAmUmCmG*mG*mA |
| | SEQ ID NO: 166 | Phos mU*fC*mCmGfAmUfGmCmUmGmCmUmGfUmGfCmCmAmCmCmA*mU*mC |
| D:61 | SEQ ID NO: 70 | mA*mG*mGmAmGmGmAmUfAfUfUmCmUmGmCmAmGmCmU*mG*mA |
| | SEQ ID NO: 167 | Phos mU*fC*mAmGfCmUfGmCmAmGmAmAmUfAmUfCmCmUmCmCmU*mC*mC |
| D:62 | SEQ ID NO: 71 | mU*mU*mGmAmGmGmUmCfUfUfAmAmAmGmGmCmUmCmA*mC*mA |
| | SEQ ID NO: 168 | Phos mU*fG*mUmGfAmGfCmCmUmUmUmAmAfGmAfCmCmUmCmAmA*mA*mU |
| D:63 | SEQ ID NO: 72 | mG*mC*mCmCmAmGmGmAfAfCfUmUmCmGmGmGmCmAmA*mG*mA |
| | SEQ ID NO: 169 | Phos mU*fC*mUmUfGmCfCmCmGmAmAmGmUfUmCfCmUmGmGmGmC*mU*mG |
| D:64 | SEQ ID NO: 73 | mU*mA*mGmAmAmGmUmCfCfAfGmCmUmGmAmGmGmAmG*mC*mA |
| | SEQ ID NO: 170 | Phos mU*fG*mCmUfCmCfUmCmAmGmCmUmGfGmAfCmUmUmCmUmA*mG*mC |
| D:65 | SEQ ID NO: 74 | mC*mU*mGmCmCmUmGmUfUfCfAmCmUmGmGmGmAmUmC*mA*mA |
| | SEQ ID NO: 171 | Phos mU*fU*mGmAfUmCfCmCmAmGmUmGmAfAmCfAmGmGmCmAmG*mC*mU |
| D:66 | SEQ ID NO: 75 | mU*mC*mUmGmCmCmUmGfGfAfUmGmGmAmAmCmUmGmA*mG*mA |
| | SEQ ID NO: 172 | Phos mU*fC*mUmCfAmGfUmUmCmCmAmUmCfCmAfGmGmCmAmGmA*mU*mU |
| D:67 | SEQ ID NO: 76 | mC*mA*mCmGmCmUmGmAfCfAfAmGmCmAmGmAmGmCmC*mA*mA |
| | SEQ ID NO: 173 | Phos mU*fU*mGmGfCmUfCmUmGmCmUmUmGfUmCfAmGmCmGmUmG*mA*mG |
| D:68 | SEQ ID NO: 77 | mU*mG*mCmCmUmGmGmAfUfGfGmAmAmCmUmGmAmGmG*mA*mA |
| | SEQ ID NO: 174 | Phos mU*fU*mCmCfUmCfAmGmUmUmCmCmAfUmCfCmAmGmGmCmA*mG*mA |
| D:69 | SEQ ID NO: 78 | mG*mA*mUmGmGmUmGmGfCfAfCmAmGmCmAmGmCmAmU*mC*mA |
| | SEQ ID NO: 175 | Phos mU*fG*mAmUfGmCfUmGmCmUmGmUmGfCmCfAmCmCmAmUmC*mU*mC |
| D:70 | SEQ ID NO: 79 | mG*mU*mCmUmUmAmAmAfGfGfCmUmCmAmCmGmCmUmG*mA*mA |
| | SEQ ID NO: 176 | Phos mU*fU*mCmAfGmCfGmUmGmAmGmCmCfUmUfUmAmAmGmAmC*mC*mU |
| D:71 | SEQ ID NO: 80 | mG*mA*mUmGmCmAmGmCfCfCfAmGmGmAmAmCmUmUmC*mG*mA |
| | SEQ ID NO: 177 | Phos mU*fC*mGmAfAmGfUmUmCmCmUmGmGfGmCfUmGmCmAmUmC*mC*mC |
| D:72 | SEQ ID NO: 81 | mG*mA*mGmAmAmUmUmUfGfAfGmGmUmCmUmUmAmAmA*mG*mA |
| | SEQ ID NO: 178 | Phos mU*fC*mUmUfUmAfAmGmAmCmCmUmCfAmAfAmUmUmCmUmC*mG*mG |
| D:73 | SEQ ID NO: 82 | mG*mA*mUmGmGmAmGmGfAfGfGmAmUmAmUmUmCmUmG*mC*mA |
| | SEQ ID NO: 179 | Phos mU*fG*mCmAfGmAfAmUmAmUmCmCmUfCmCfUmCmCmAmUmC*mU*mG |
| D:74 | SEQ ID NO: 83 | mU*mG*mAmCmAmAmAmGfGfCfCmAmGmGmAmAmCmAmG*mC*mA |
| | SEQ ID NO: 180 | Phos mU*fG*mCmUfGmUfUmCmCmUmGmGmCfCmUfUmUmGmUmCmA*mG*mC |
| D:75 | SEQ ID NO: 84 | mC*mU*mCmAmCmGmCmUfGfAfCmAmAmGmCmAmGmAmG*mC*mA |
| | SEQ ID NO: 181 | Phos mU*fG*mCmUfCmUfGmCmUmUmGmUmCfAmGfCmGmUmGmAmG*mC*mC |
| D:76 | SEQ ID NO: 85 | mG*mC*mCmUmGmAmAmUfCfUfGmCmCmUmGmGmAmUmG*mG*mA |
| | SEQ ID NO: 182 | Phos mU*fC*mCmAfUmCfCmAmGmGmCmAmGfAmUfUmCmAmGmGmC*mU*mG |
| D:77 | SEQ ID NO: 86 | mA*mC*mAmGmCmCmUmGfGfGfUmCmUmCmUmAmUmGmG*mC*mA |
| | SEQ ID NO: 183 | Phos mU*fG*mCmCfAmUfAmGmAmGmAmCmCfCmAfGmGmCmUmGmU*mU*mC |
| D:78 | SEQ ID NO: 87 | mC*mA*mGmGmAmAmCmUfUfCfGmGmGmCmAmAmGmCmC*mU*mA |
| | SEQ ID NO: 184 | Phos mU*fA*mGmGfCmUfUmGmCmCmCmGmAfAmGfUmUmCmCmUmG*mG*mG |
| D:79 | SEQ ID NO: 88 | mG*mC*mAmAmGmGmAmAfCfAfCmUmUmCmCmAmCmGmC*mC*mA |
| | SEQ ID NO: 185 | Phos mU*fG*mGmCfGmUfGmGmAmAmGmUmGfUmUfCmCmUmUmGmC*mA*mG |
| D:80 | SEQ ID NO: 89 | mU*mU*mUmGmAmGmGmUfCfUfUmAmAmAmGmGmCmUmC*mA*mA |
| | SEQ ID NO: 186 | Phos mU*fU*mGmAfGmCfCmUmUmUmAmAmGfAmCfCmUmCmAmAmA*mU*mU |
| D:81 | SEQ ID NO: 90 | mG*mG*mCmCmCmAmGmGfCfAfCmAmGmAmAmGmGmUmG*mC*mA |
| | SEQ ID NO: 187 | Phos mU*fG*mCmAfCmCfUmUmCmUmGmUmGfCmCfUmGmGmGmCmC*mA*mC |
| D:82 | SEQ ID NO: 91 | mU*mC*mAmGmAmUmGmGfAfGfGmAmGmGmAmUmAmUmU*mC*mA |
| | SEQ ID NO: 188 | Phos mU*fG*mAmAfUmAfUmCmCmUmCmCmUfCmCfAmUmCmUmGmA*mG*mU |
| D:83 | SEQ ID NO: 92 | mC*mC*mAmGmGmAmAmCfUfUfCmGmGmGmCmAmAmGmC*mC*mA |
| | SEQ ID NO: 189 | Phos mU*fG*mGmCfUmUfGmCmCmCmGmAmAfGmUfUmCmCmUmGmG*mG*mC |
| D:84 | SEQ ID NO: 93 | mC*mA*mGmCmCmCmAmGfGfAfAmCmUmUmCmGmGmGmC*mA*mA |
| | SEQ ID NO: 190 | Phos mU*fU*mGmCfCmCfGmAmAmGmUmUmCfCmUfGmGmGmCmUmG*mC*mA |
| D:85 | SEQ ID NO: 94 | mC*mC*mCmAmGmCmCmUfGfAfAmUmCmUmGmCmCmUmG*mG*mA |
| | SEQ ID NO: 191 | Phos mU*fC*mCmAfGmGfCmAmGmAmUmUmCfAmGfGmCmUmGmGmG*mA*mG |
| D:86 | SEQ ID NO: 95 | mC*mC*mCmAmGmGmAmAfCfUfUmCmGmGmGmCmAmAmG*mC*mA |
| | SEQ ID NO: 192 | Phos mU*fG*mCmUfUmGfCmCmCmGmAmAmGfUmUfCmCmUmGmGmG*mC*mU |
| D:87 | SEQ ID NO: 96 | mC*mA*mUmCmGmGmCmUfGfCfGmAmCmAmGmAmUmCmC*mA*mA |
| | SEQ ID NO: 193 | Phos mU*fU*mGmGfAmUfCmUmGmUmCmGmCfAmGfCmCmGmAmUmG*mC*mU |
| D:88 | SEQ ID NO: 97 | mG*mU*mGmGmCmAmCmAfGfCfAmGmCmAmUmCmGmGmC*mU*mA |
| | SEQ ID NO: 194 | Phos mU*fA*mGmCfCmGfAmUmGmCmUmGmCfUmGfUmGmCmCmAmC*mC*mA |
| D:89 | SEQ ID NO: 98 | mG*mG*mCmCmCmUmCmAfCfAfGmGmCmCmAmCmGmUmG*mC*mA |
| | SEQ ID NO: 195 | Phos mU*fG*mCmAfCmGfUmGmGmCmCmUmGfUmGfAmGmGmGmCmC*mC*mA |
| D:90 | SEQ ID NO: 99 | mA*mA*mCmUmUmCmGmGfGfCfAmAmGmCmCmUmGmUmU*mG*mA |
| | SEQ ID NO: 196 | Phos mU*fC*mAmAfCmAfGmGmCmUmUmGmCfCmCfGmAmAmGmUmU*mC*mC |
| D:91 | SEQ ID NO: 100 | mU*mA*mAmAmGmGmCmUfCfAfCmGmCmUmGmAmCmAmA*mG*mA |
| | SEQ ID NO: 197 | Phos mU*fC*mUmUfGmUfCmAmGmCmGmUmGfAmGfCmCmUmUmUmA*mA*mG |
| D:92 | SEQ ID NO: 101 | mU*mU*mCmGmGmGmCmAfAfGfCmCmUmGmUmUmGmGmA*mG*mA |
| | SEQ ID NO: 198 | Phos mU*fC*mUmCfCmAfAmCmAmGmGmCmUfUmGfCmCmCmGmAmA*mG*mU |
| D:93 | SEQ ID NO: 102 | mA*mG*mCmAmUmCmGmGfCfUfGmCmGmAmCmAmGmAmU*mC*mA |
| | SEQ ID NO: 199 | Phos mU*fG*mAmUfCmUfGmUmCmGmCmAmGfCmCfGmAmUmGmCmU*mG*mC |
| D:94 | SEQ ID NO: 103 | mA*mU*mCmUmGmCmCmUfGfGfAmUmGmGmAmAmCmUmG*mA*mA |
| | SEQ ID NO: 200 | Phos mU*fU*mCmAfGmUfUmCmCmAmUmCmCfAmGfGmCmAmGmAmU*mU*mC |
| D:95 | SEQ ID NO: 104 | mG*mC*mUmCmAmCmGmCfUfGfAmCmAmAmGmCmAmGmA*mG*mA |
| | SEQ ID NO: 201 | Phos mU*fC*mUmCfUmGfCmUmUmGmUmCmAfGmCfGmUmGmAmGmC*mC*mU |
| D:96 | SEQ ID NO: 105 | mA*mG*mAmUmGmGmAmGfGfAfGmGmAmUmAmUmUmCmU*mG*mA |
| | SEQ ID NO: 202 | Phos mU*fC*mAmGfAmAfUmAmUmCmCmUmCfCmUfCmCmAmUmCmU*mG*mA |
| D:97 | SEQ ID NO: 106 | mG*mC*mAmCmAmGmCmAfGfCfAmUmCmGmGmCmUmGmC*mG*mA |
| | SEQ ID NO: 203 | Phos mU*fC*mGmCfAmGfCmCmGmAmUmGmCfUmGfCmUmGmUmGmC*mC*mA |
| D:98 | SEQ ID NO: 107 | mA*mG*mGmAmAmCmAmGfCfCfUmGmGmGmUmCmUmCmU*mA*mA |
| | SEQ ID NO: 204 | Phos mU*fU*mAmGfAmGfAmCmCmCmAmGmGfCmUfGmUmUmCmCmU*mG*mG |
| D:99 | SEQ ID NO: 108 | mG*mG*mAmGmAmUmGmGfUf?fGmCmAmCmAmGmCmAmG*mC*mA |
| | SEQ ID NO: 205 | Phos mU*fG*mCmUfGmCfUmGmUmGmCmCmAfCmCfAmUmCmUmCmC*mC*mG |
| D:100 | SEQ ID NO: 109 | mG*mC*mAmGmCmAmUmCfGfGfCmUmGmCmGmAmCmAmG*mA*mA |
| | SEQ ID NO: 206 | Phos mU*fU*mCmUfGmUfCmGmCmAmGmCmCfGmAfUmGmCmUmGmC*mU*mG |
| D:101 | SEQ ID NO: 110 | mA*mA*mUmUmUmGmAmGfGfUfCmUmUmAmAmAmGmGmC*mU*mA |
| | SEQ ID NO: 207 | Phos mU*fA*mGmCfCmUfUmUmAmAmGmAmCfCmUfCmAmAmAmUmU*mC*mU |
| D:102 | SEQ ID NO: 111 | mA*mA*mGmUmCmCmAmGfCfUfGmAmGmGmAmGmCmGmC*mC*mA |
| | SEQ ID NO: 208 | Phos mU*fG*mGmCfGmCfUmCmCmUmCmAmGfCmUfGmGmAmCmUmU*mC*mU |
| D:103 | SEQ ID NO: 112 | mG*mA*mGmGmUmCmUmUfAfAfAmGmGmCmUmCmAmCmG*mC*mA |
| | SEQ ID NO: 209 | Phos mU*fG*mCmGfUmGfAmGmCmCmUmUmUfAmAfGmAmCmCmUmC*mA*mA |

P or [Phos] indicates a 5′ phosphate
m indicates 2′O-methyl modified ribose on the listed nucleotide
f indicates 2′F modified ribose on the listed nucleotide
* indicates a phosphorothioate bond (in place of a phosphodiester bond)

TABLE 3B
Modified sense and antisense strands for the GalNAc-ANGPTL8
RNAi Agents herein

| | | Sequence (5′ to 3′ for each sense strand |
|---|---|---|
| Duplex | SEQ ID NO: | (SS) and antisense strand (AS)) |
| D:109 | SEQ ID NO: 16 | mU*mC*mGmGmGmCmAmAfGfCfCmUmGmUmUmGmGmAmG*mA*mA |
| | SEQ ID NO: 210 | mU*fU*mCmUfCmCfAmAmCmAmGmGmCfUmUfGmCmCmCmGmA*mA*mG |
| D:110 | SEQ ID NO: 17 | mA*mA*mUmCmUmGmCmCfUfGfGmAmUmGmGmAmAmCmU*mG*mA |
| | SEQ ID NO: 211 | mU*fC*mAmGfUmUfCmCmAmUmCmCmAfGmGfCmAmGmAmUmU*mC*mA |
| D:111 | SEQ ID NO: 18 | mC*mC*mUmGmGmAmUmGfGfAfAmCmUmGmAmGmGmAmC*mC*mA |
| | SEQ ID NO: 212 | mU*fG*mGmUfCmCfUmCmAmGmUmUmCfCmAfUmCmCmAmGmG*mC*mA |
| D:112 | SEQ ID NO: 19 | mU*mU*mAmAmAmGmCmAfGfAfGmUmCmGmUmGmGmCmA*mU*mA |
| | SEQ ID NO: 213 | mU*fA*mUmGfCmCfAmCmGmAmCmUmCfUmGfCmUmUmUmAmA*mU*mG |
| D:113 | SEQ ID NO: 20 | mUmG*mCmGmAmCmAmGfAfUfCmCmAmGmGmAmGmAmG*mA*mA |
| | SEQ ID NO: 214 | mU*fU*mCmUfCmUfCmCmUmGmGmAmUfCmUfGmUmCmGmCmA*mG*mC |
| D:114 | SEQ ID NO: 21 | mU*mG*mAmAmUmCmUmGfCfCfUmGmGmAmUmGmGmAmA*mC*mA |
| | SEQ ID NO: 215 | mU*fG*mUmUfCmCfAmUmCmCmAmGmGfCmAfGmAmUmUmCmA*mG*mG |
| D:115 | SEQ ID NO: 22 | mG*mA*mGmCmUmGmCmCfUfGfUmUmCmAmCmUmGmGmG*mA*mA |
| | SEQ ID NO: 216 | mU*fU*mCmCfCmAfGmUmGmAmAmCmAfGmGfCmAmGmCmUmC*mC*mU |
| D:116 | SEQ ID NO: 23 | mA*mG*mGmUmCmUmUmAfAfAfGmGmCmUmCmAmCmGmC*mU*mA |
| | SEQ ID NO: 217 | mU*fA*mGmCfGmUfGmAmGmCmCmUmUfUmAfAmGmAmCmCmU*mC*mA |
| D:117 | SEQ ID NO: 24 | mU*mC*mAmCmUmGmGmGfAfUfCmAmGmCmCmAmGmGmG*mC*mA |
| | SEQ ID NO: 218 | mU*fG*mCmCfCmUfGmGmCmUmGmAmUfCmCfCmAmGmUmGmA*mA*mC |
| D:118 | SEQ ID NO: 25 | mU*mC*mAmUmUmAmAmAfGfCfAmGmAmGmUmCmGmUmG*mG*mA |
| | SEQ ID NO: 219 | mU*fC*mCmAfCmGfAmCmUmCmUmGmCfUmUfUmAmAmUmGmA*mG*mG |
| D:119 | SEQ ID NO: 26 | mG*mA*mAmCmAmGmCmCfUfGfGmGmUmCmUmCmUmAmU*mG*mA |
| | SEQ ID NO: 220 | mU*fC*mAmUfAmGfAmGmAmCmCmCmAfGmGfCmUmGmUmUmC*mC*mU |
| D:120 | SEQ ID NO: 27 | mG*mG*mGmGmCmAmGmGfAfGfGmUmCmAmGmCmCmGmG*mG*mA |
| | SEQ ID NO: 221 | mU*fC*mCmCfGmGfCmUmGmAmCmCmUfCmCfUmGmCmCmCmC*mA*mG |
| D:121 | SEQ ID NO: 28 | mA*mG*mCmGmGmCmUmAfGfAfAmGmUmCmCmAmGmCmU*mG*mA |
| | SEQ ID NO: 222 | mU*fC*mAmGfCmUfGmGmAmCmUmUmCfUmAfGmCmCmGmCmU*mG*mC |
| D:122 | SEQ ID NO: 29 | mG*mA*mGmAmUmGmGmUfGfGfCmAmCmAmGmCmAmGmC*mA*mA |
| | SEQ ID NO: 223 | mU*fU*mGmCfUmGfCmUmGmUmGmCmCfAmCfCmAmUmCmUmC*mC*mC |
| D:123 | SEQ ID NO: 30 | mG*mG*mCmUmGmAmCmAfAfAfGmGmCmCmAmGmGmAmA*mC*mA |
| | SEQ ID NO: 224 | mU*fG*mUmUfCmCfUmGmGmCmCmUmUfUmGfUmCmAmGmCmC*mG*mU |
| D:124 | SEQ ID NO: 31 | mG*mU*mCmCmAmGmCmUfGfAfGmGmAmGmCmGmCmCmU*mG*mA |
| | SEQ ID NO: 225 | mU*fC*mAmGfGmCfGmCmUmCmCmUmCfAmGfCmUmGmGmAmC*mU*mU |
| D:125 | SEQ ID NO: 32 | mC*mA*mUmUmAmAmAmGfCfAfGmAmGmUmCmGmUmGmG*mC*mA |
| | SEQ ID NO: 226 | mU*fG*mCmCfAmCfGmAmCmUmCmUmGfCmUfUmUmAmAmUmG*mA*mG |
| D:126 | SEQ ID NO: 33 | mU*mC*mCmCmAmGmCmCfUfGfAmAmUmCmUmGmCmCmU*mG*mA |
| | SEQ ID NO: 227 | mU*fC*mAmGfGmCfAmGmAmUmUmCmAfGmGfCmUmGmGmGmA*mG*mC |
| D:127 | SEQ ID NO: 34 | mG*mG*mAmUmGmCmAmGfCfCfCmAmGmGmAmAmCmUmU*mC*mA |
| | SEQ ID NO: 228 | mU*fG*mAmAfGmUfUmCmCmUmGmGmGfCmUfGmCmAmUmCmC*mC*mG |
| D:128 | SEQ ID NO: 35 | mG*mC*mUmAmGmAmAmGfUfCfCmAmGmCmUmGmAmGmG*mA*mA |
| | SEQ ID NO: 229 | mU*fU*mCmCfUmCfAmGmCmUmGmGmAfCmUfUmCmUmAmGmC*mC*mG |
| D:129 | SEQ ID NO: 36 | mC*mA*mCmAmGmCmAmGfCfAfUmCmGmGmCmUmGmCmG*mA*mA |
| | SEQ ID NO: 230 | mU*fU*mCmGfCmAfGmCmCmGmAmUmGfCmUfGmCmUmGmUmG*mC*mC |
| D:130 | SEQ ID NO: 37 | mG*mG*mAmAmCmUmUmCfGfGfGmCmAmAmGmCmCmUmG*mU*mA |
| | SEQ ID NO: 231 | mU*fA*mCmAfGmGfCmUmUmGmCmCmCfGmAfAmGmUmUmCmC*mU*mG |
| D:131 | SEQ ID NO: 38 | mA*mC*mUmGmGmGmAmUfCfAfGmCmCmAmGmGmGmCmG*mC*mA |
| | SEQ ID NO: 232 | mU*fG*mCmGfCmCfCmUmGmGmCmUmGfAmUfCmCmCmAmGmU*mG*mA |
| D:132 | SEQ ID NO: 39 | mA*mG*mAmAmGmUmCmCfAfGfCmUmGmAmGmGmAmGmC*mG*mA |
| | SEQ ID NO: 233 | mU*fC*mGmCfUmCfCmUmCmAmGmCmUfGmGfAmCmUmUmCmU*mA*mG |
| D:133 | SEQ ID NO: 40 | mG*mC*mAmUmCmGmGmCfUfGfCmGmAmCmAmGmAmUmC*mC*mA |
| | SEQ ID NO: 234 | mU*fG*mGmAfUmCfUmGmUmCmGmCmAfGmCfCmGmAmUmGmC*mU*mG |
| D:134 | SEQ ID NO: 41 | mA*mG*mAmUmGmGmUmGfGfCfAmCmAmGmCmAmGmCmA*mU*mA |
| | SEQ ID NO: 235 | mU*fA*mUmGfCmUfGmCmUmGmUmGmCfCmAfCmCmAmUmCmU*mC*mC |
| D:135 | SEQ ID NO: 42 | mG*mG*mUmGmGmCmAmCfAfGfCmAmGmCmAmUmCmGmG*mC*mA |
| | SEQ ID NO: 236 | mU*fG*mCmCfGmAfUmGmCmUmGmCmUfGmUfGmCmCmAmCmC*mA*mU |
| D:136 | SEQ ID NO: 43 | mA*mC*mAmGmCmAmGmCfAfUfCmGmGmCmUmGmCmGmA*mC*mA |
| | SEQ ID NO: 237 | mU*fG*mUmCfGmCfAmGmCmCmGmAmUfGmCfUmGmCmUmGmU*mG*mC |
| D:137 | SEQ ID NO: 44 | mA*mA*mGmGmCmUmCmAfCfGfCmUmGmAmCmAmAmGmC*mA*mA |
| | SEQ ID NO: 238 | mU*fU*mGmCfUmUfGmUmCmAmGmCmGfUmGfAmGmCmCmUmU*mU*mA |
| D:138 | SEQ ID NO: 45 | mA*mG*mCmCmUmGmAmAfUfCfUmGmCmCmUmGmGmAmU*mG*mA |
| | SEQ ID NO: 239 | mU*fC*mAmUfCmCfAmGmGmCmAmGmAfUmUfCmAmGmGmCmU*mG*mG |
| D:139 | SEQ ID NO: 46 | mA*mU*mCmGmGmCmUmGfCfGfAmCmAmGmAmUmCmCmA*mG*mA |
| | SEQ ID NO: 240 | mU*fC*mUmGfGmAfUmCmUmGmUmCmGfCmAfGmCmCmGmAmU*mG*mC |
| D:140 | SEQ ID NO: 47 | mA*mG*mUmCmCmAmGmCfUfGfAmGmGmAmGmCmGmCmC*mU*mA |
| | SEQ ID NO: 241 | mU*fA*mGmGfCmGfCmUmCmCmUmCmAfGmCfUmGmGmAmCmU*mU*mC |
| D:141 | SEQ ID NO: 48 | mC*mG*mGmCmAmGmAmGfGfCfGmGmGmAmGmAmUmGmG*mU*mA |
| | SEQ ID NO: 241 | mU*fA*mGmGfCmGfCmUmCmCmUmCmAfGmCfUmGmGmAmCmU*mU*mC |
| D:142 | SEQ ID NO: 49 | mA*mUmGmCmUmGmCmAfAfGfGmAmAmCmAmCmUmUmC*mC*mA |
| | SEQ ID NO: 243 | mU*fG*mGmAfAmGfUmGmUmUmCmCmUfUmGfCmAmGmCmAmU*mG*mA |
| D:143 | SEQ ID NO: 50 | mC*mU*mUmCmGmGmGmCfAfAfGmCmCmUmGmUmUmGmG*mA*mA |
| | SEQ ID NO: 244 | mU*fU*mCmCfAmAfCmAmGmGmCmUmUfGmCfCmCmGmAmAmG*mU*mU |
| D:144 | SEQ ID NO: 51 | mG*mG*mAmGmCmUmGmCfCfUfGmUmUmCmAmCmUmGmG*mG*mA |
| | SEQ ID NO: 245 | mU*fC*mCmCfAmGfUmGmAmAmCmAmGfGmCfAmGmCmUmCmC*mU*mC |
| D:145 | SEQ ID NO: 52 | mG*mC*mCmUmGmGmAmUfGfGfAmAmCmUmGmAmGmGmA*mC*mA |
| | SEQ ID NO: 246 | mU*fG*mUmCfCmUfCmAmGmUmUmCmCfAmUfCmCmAmGmGmC*mA*mG |
| D:146 | SEQ ID NO: 53 | mC*mA*mAmGmGmAmAmCfAfCfUmUmCmCmAmCmGmCmC*mC*mA |
| | SEQ ID NO: 247 | mU*fG*mGmGfCmGfUmGmGmAmAmGmUfGmUfUmCmCmUmUmG*mC*mA |
| D:147 | SEQ ID NO: 54 | mG*mG*mCmUmAmGmAmAfGfUfCmCmAmGmCmUmGmAmG*mG*mA |
| | SEQ ID NO: 248 | mU*fC*mCmUfCmAfGmCmUmGmGmAmCfUmUfCmUmAmGmCmC*mG*mC |
| D:148 | SEQ ID NO: 55 | mU*mG*mAmGmGmUmCmUfUfAfAmAmGmGmCmUmCmAmC*mG*mA |
| | SEQ ID NO: 249 | mU*fC*mGmUfGmAfGmCmCmUmUmUmAfAmGfAmCmCmUmCmA*mA*mA |
| D:149 | SEQ ID NO: 56 | mG*mG*mGmCmAmAmGmCfCfUfGmUmUmGmGmAmGmAmC*mU*mA |
| | SEQ ID NO: 250 | mU*fA*mGmUfCmUfCmCmAmAmCmAmGfGmCfUmUmGmCmCmC*mG*mA |
| D:150 | SEQ ID NO: 57 | mG*mC*mGmGmCmUmAmGfAfAfGmUmCmCmAmGmCmUmG*mA*mA |
| | SEQ ID NO: 251 | mU*fU*mCmAfGmCfUmGmGmAmCmUmUfCmUfAmGmCmCmGmC*mU*mG |
| D:151 | SEQ ID NO: 58 | mU*mG*mGmCmAmCmAmGfCfAfGmCmAmUmCmGmGmCmU*mG*mA |
| | SEQ ID NO: 252 | mU*fC*mAmGfCmCfGmAmUmGmCmUmGfCmUfGmUmGmCmCmA*mC*mC |
| D:152 | SEQ ID NO: 59 | mA*mU*mGmGmUmGmGmCfAfCfAmGmCmAmGmCmAmUmC*mG*mA |
| | SEQ ID NO: 253 | mU*fC*mGmAfUmGfCmUmGmCmUmGmUfGmCfCmAmCmCmAmU*mC*mU |
| D:153 | SEQ ID NO: 60 | mA*mG*mAmAmUmUmUmGfAfGfGmUmCmUmUmAmAmAmG*mG*mA |
| | SEQ ID NO: 254 | mU*fC*mCmUfUmUfAmAmGmAmCmCmUfCmAfAmAmUmUmCmU*mC*mG |
| D:154 | SEQ ID NO: 61 | mG*mG*mAmAmCmAmGmCfCfUfGmGmGmUmCmUmCmUmA*mU*mA |
| | SEQ ID NO: 255 | mU*fA*mUmAfGmAfGmAmCmCmCmAmGfGmCfUmGmUmUmCmC*mU*mG |
| D:155 | SEQ ID NO: 62 | mA*mG*mGmCmUmCmAmCfGfCfUmGmAmCmAmAmGmCmA*mG*mA |
| | SEQ ID NO: 256 | mU*fC*mUmGfCmUfUmGmUmCmAmGmCfGmUfGmAmGmCmCmU*mU*mU |
| D:156 | SEQ ID NO: 63 | mG*mG*mCmAmCmAmGmCfAfGfCmAmUmCmGmGmCmUmG*mC*mA |
| | SEQ ID NO: 257 | mU*fG*mCmAfGmCfCmGmAmUmGmCmUfGmCfUmGmUmGmCmC*mA*mC |
| D:157 | SEQ ID NO: 64 | mC*mG*mGmGmCmAmAmGfCfCfUmGmUmUmGmGmAmGmA*mC*mA |
| | SEQ ID NO: 258 | mU*fG*mUmCfUmCfCmAmAmCmAmGmGfCmUfUmGmCmCmCmG*mA*mA |
| D:158 | SEQ ID NO: 65 | mA*mA*mAmGmGmCmUmCfAfCfGmCmUmGmAmCmAmAmG*mC*mA |
| | SEQ ID NO: 259 | mU*fG*mCmUfUmGfUmCmAmGmCmGmUfGmAfGmCmCmUmUmU*mA*mA |
| D:159 | SEQ ID NO: 66 | mG*mA*mAmUmCmUmGmCfCfUfGmGmAmUmGmGmAmAmC*mU*mA |
| | SEQ ID NO: 260 | mU*fA*mGmUfUmCfCmAmUmCmCmAmGfGmCfAmGmAmUmUmC*mA*mG |
| D:160 | SEQ ID NO: 67 | mC*mC*mUmGmUmUmCmAfCfUfGmGmGmAmUmCmAmGmC*mC*mA |
| | SEQ ID NO: 261 | mU*fG*mGmCfUmGfAmUmCmCmCmAmGfUmGfAmAmCmAmGmG*mC*mA |
| D:161 | SEQ ID NO: 68 | mA*mU*mUmAmAmAmGmCfAfGfAmGmUmCmGmUmGmGmC*mA*mA |
| | SEQ ID NO: 262 | mU*fU*mGmCfCmAfCmGmAmCmUmCmUfGmCfUmUmUmAmAmU*mG*mA |
| D:162 | SEQ ID NO: 69 | mU*mG*mGmUmGmGmCmAfCfAfGmCmAmGmCmAmUmCmG*mG*mA |
| | SEQ ID NO: 263 | mU*fC*mCmGfAmUfGmCmUmGmCmUmGfUmGfCmCmAmCmCmA*mU*mC |
| D:163 | SEQ ID NO: 70 | mA*mG*mGmAmGmGmAmUfAfUfUmCmUmGmCmAmGmCmU*mG*mA |
| | SEQ ID NO: 264 | mU*fC*mAmGfCmUfGmCmAmGmAmAmUfAmUfCmCmUmCmCmU*mC*mC |
| D:164 | SEQ ID NO: 71 | mU*mU*mGmAmGmGmUmCfUfUfAmAmAmGmGmCmUmCmA*mC*mA |
| | SEQ ID NO: 265 | mU*fG*mUmGfAmGfCmCmUmUmUmAmAfGmAfCmCmUmCmAmA*mA*mU |
| D:165 | SEQ ID NO: 72 | mG*mC*mCmCmAmGmGmAfAfCfUmUmCmGmGmGmCmAmA*mG*mA |
| | SEQ ID NO: 266 | mU*fC*mUmUfGmCfCmCmGmAmAmGmUfUmCfCmUmGmGmGmC*mU*mG |
| D:166 | SEQ ID NO: 73 | mU*mA*mGmAmAmGmUmCfCfAfGmCmUmGmAmGmGmAmG*mC*mA |
| | SEQ ID NO: 267 | mU*fG*mCmUfCmCfUmCmAmGmCmUmGfGmAfCmUmUmCmUmA*mG*mC |
| D:167 | SEQ ID NO: 74 | mC*mU*mGmCmCmUmGmUfUfCfAmCmUmGmGmGmAmUmC*mA*mA |
| | SEQ ID NO: 268 | mU*fU*mGmAfUmCfCmCmAmGmUmGmAfAmCfAmGmGmCmAmG*mC*mU |
| D:168 | SEQ ID NO: 75 | mU*mC*mUmGmCmCmUmGfGfAfUmGmGmAmAmCmUmGmA*mG*mA |
| | SEQ ID NO: 269 | mU*fC*mUmCfAmGfUmUmCmCmAmUmCfCmAfGmGmCmAmGmA*mU*mU |
| D:169 | SEQ ID NO: 76 | mC*mA*mCmGmCmUmGmAfCfAfAmGmCmAmGmAmGmCmC*mA*mA |
| | SEQ ID NO: 270 | mU*fU*mGmGfCmUfCmUmGmCmUmUmGfUmCfAmGmCmGmUmG*mA*mG |
| D:170 | SEQ ID NO: 77 | mU*mG*mCmCmUmGmGmAfUfGfGmAmAmCmUmGmAmGmG*mA*mA |
| | SEQ ID NO: 271 | mU*fU*mCmCfUmCfAmGmUmUmCmCmAfUmCfCmAmGmGmCmA*mG*mA |
| D:171 | SEQ ID NO: 78 | mG*mA*mUmGmGmUmGmGfCfAfCmAmGmCmAmGmCmAmU*mC*mA |
| | SEQ ID NO: 272 | mU*fG*mAmUfGmCfUmGmCmUmGmUmGfCmCfAmCmCmAmUmC*mU*mC |
| D:172 | SEQ ID NO: 79 | mG*mU*mCmUmUmAmAmAfGfGfCmUmCmAmCmGmCmUmG*mA*mA |
| | SEQ ID NO: 273 | mU*fU*mCmAfGmCfGmUmGmAmGmCmCfUmUfUmAmAmGmAmC*mC*mU |
| D:173 | SEQ ID NO: 80 | mG*mA*mUmGmCmAmGmCfCfCfAmGmGmAmAmCmUmUmC*mG*mA |
| | SEQ ID NO: 274 | mU*fC*mGmAfAmGfUmUmCmCmUmGmGfGmCfUmGmCmAmUmC*mC*mC |
| D:174 | SEQ ID NO: 81 | mG*mA*mGmAmAmUmUmUfGfAfGmGmUmCmUmUmAmAmA*mG*mA |
| | SEQ ID NO: 275 | mU*fC*mUmUfUmAfAmGmAmCmCmUmCfAmAfAmUmUmCmUmC*mG*mG |
| D:175 | SEQ ID NO: 82 | mG*mA*mUmGmGmAmGmGfAfGfGmAmUmAmUmUmCmUmG*mC*mA |
| | SEQ ID NO: 276 | mU*fG*mCmAfGmAfAmUmAmUmCmCmUfCmCfUmCmCmAmUmC*mU*mG |
| D:176 | SEQ ID NO: 83 | mU*mG*mAmCmAmAmAmGfGfCfCmAmGmGmAmAmCmAmG*mC*mA |
| | SEQ ID NO: 277 | mU*fG*mCmUfGmUfUmCmCmUmGmGmCfCmUfUmUmGmUmCmA*mG*mC |
| D:177 | SEQ ID NO: 84 | mC*mU*mCmAmCmGmCmUfGfAfCmAmAmGmCmAmGmAmG*mC*mA |
| | SEQ ID NO: 278 | mU*fG*mCmUfCmUfGmCmUmUmGmUmCfAmGfCmGmUmGmAmG*mC*mC |
| D:178 | SEQ ID NO: 85 | mG*mC*mCmUmGmAmAmUfCfUfGmCmCmUmGmGmAmUmG*mG*mA |
| | SEQ ID NO: 279 | mU*fC*mCmAfUmCfCmAmGmGmCmAmGfAmUfUmCmAmGmGmC*mU*mG |
| D:179 | SEQ ID NO: 86 | mA*mC*mAmGmCmCmUmGfGfGfUmCmUmCmUmAmUmGmG*mC*mA |
| | SEQ ID NO: 280 | mU*fG*mCmCfAmUfAmGmAmGmAmCmCfCmAfGmGmCmUmGmU*mU*mC |
| D:180 | SEQ ID NO: 87 | mC*mA*mGmGmAmAmCmUfUfCfGmGmGmCmAmAmGmCmC*mU*mA |
| | SEQ ID NO: 281 | mU*fA*mGmGfCmUfUmGmCmCmCmGmAfAmGfUmUmCmCmUmG*mG*mG |
| D:181 | SEQ ID NO: 88 | mG*mC*mAmAmGmGmAmAfCfAfCmUmUmCmCmAmCmGmC*mC*mA |
| | SEQ ID NO: 282 | mU*fG*mGmCfGmUfGmGmAmAmGmUmGfUmUfCmCmUmUmGmC*mA*mG |
| D:182 | SEQ ID NO: 89 | mU*mUmUmGmAmGmGmUfCfUfUmAmAmAmGmGmCmUmC*mA*mA |
| | SEQ ID NO: 283 | mU*fU*mGmAfGmCfCmUmUmUmAmAmGfAmCfCmUmCmAmAmA*mU*mU |
| D:183 | SEQ ID NO: 90 | mG*mG*mCmCmCmAmGmGfCfAfCmAmGmAmAmGmGmUmG*mC*mA |
| | SEQ ID NO: 284 | mU*fG*mCmAfCmCfUmUmCmUmGmUmGfCmCfUmGmGmGmCmC*mA*mC |
| D:184 | SEQ ID NO: 91 | mU*mC*mAmGmAmUmGmGfAfGfGmAmGmGmAmUmAmUmU*mC*mA |
| | SEQ ID NO: 285 | mU*fG*mAmAfUmAfUmCmCmUmCmCmUfCmCfAmUmCmUmGmA*mG*mU |
| D:185 | SEQ ID NO: 92 | mC*mC*mAmGmGmAmAmCfUfUfCmGmGmGmCmAmAmGmC*mC*mA |
| | SEQ ID NO: 286 | mU*fG*mGmCfUmUfGmCmCmCmGmAmAfGmUfUmCmCmUmGmG*mG*mC |
| D:186 | SEQ ID NO: 93 | mC*mA*mGmCmCmCmAmGfGfAfAmCmUmUmCmGmGmGmC*mA*mA |
| | SEQ ID NO: 287 | mU*fU*mGmCfCmCfGmAmAmGmUmUmCfCmUfGmGmGmCmUmG*mC*mA |
| D:187 | SEQ ID NO: 94 | mC*mC*mCmAmGmCmCmUfGfAfAmUmCmUmGmCmCmUmG*mG*mA |
| | SEQ ID NO: 288 | mU*fC*mCmAfGmGfCmAmGmAmUmUmCfAmGfGmCmUmGmGmG*mA*mG |
| D:188 | SEQ ID NO: 95 | mC*mC*mCmAmGmGmAmAfCfUfUmCmGmGmGmCmAmAmG*mC*mA |
| | SEQ ID NO: 289 | mU*fG*mCmUfUmGfCmCmCmGmAmAmGfUmUfCmCmUmGmGmG*mC*mU |
| D:189 | SEQ ID NO: 96 | mC*mA*mUmCmGmGmCmUfGfCfGmAmCmAmGmAmUmCmC*mA*mA |
| | SEQ ID NO: 290 | mU*fU*mGmGfAmUfCmUmGmUmCmGmCfAmGfCmCmGmAmUmG*mC*mU |
| D:190 | SEQ ID NO: 97 | mG*mU*mGmGmCmAmCmAfGfCfAmGmCmAmUmCmGmGmC*mU*mA |
| | SEQ ID NO: 291 | mU*fA*mGmCfCmGfAmUmGmCmUmGmCfUmGfUmGmCmCmAmC*mC*mA |
| D:191 | SEQ ID NO: 98 | mG*mG*mCmCmCmUmCmAfCfAfGmGmCmCmAmCmGmUmG*mC*mA |
| | SEQ ID NO: 292 | mU*fG*mCmAfCmGfUmGmGmCmCmUmGfUmGfAmGmGmGmCmC*mC*mA |
| D:192 | SEQ ID NO: 99 | mA*mA*mCmUmUmCmGmGfGfCfAmAmGmCmCmUmGmUmU*mG*mA |
| | SEQ ID NO: 293 | mU*fC*mAmAfCmAfGmGmCmUmUmGmCfCmCfGmAmAmGmUmU*mC*mC |
| D:193 | SEQ ID NO: 100 | mU*mA*mAmAmGmGmCmUfCfAfCmGmCmUmGmAmCmAmA*mG*mA |
| | SEQ ID NO: 294 | mU*fC*mUmUfGmUfCmAmGmCmGmUmGfAmGfCmCmUmUmUmA*mA*mG |
| D:194 | SEQ ID NO: 101 | mU*mU*mCmGmGmGmCmAfAfGfCmCmUmGmUmUmGmGmA*mG*mA |
| | SEQ ID NO: 295 | mU*fC*mUmCfCmAfAmCmAmGmGmCmUfUmGfCmCmCmGmAmA*mG*mU |
| D:195 | SEQ ID NO: 102 | mA*mG*mCmAmUmCmGmGfCfUfGmCmGmAmCmAmGmAmU*mC*mA |
| | SEQ ID NO: 296 | mU*fG*mAmUfCmUfGmUmCmGmCmAmGfCmCfGmAmUmGmCmU*mG*mC |
| D:196 | SEQ ID NO: 103 | mA*mU*mCmUmGmCmCmUfGfGfAmUmGmGmAmAmCmUmG*mA*mA |
| | SEQ ID NO: 297 | mU*fU*mCmAfGmUfUmCmCmAmUmCmCfAmGfGmCmAmGmAmU*mU*mC |
| D:197 | SEQ ID NO: 104 | mG*mC*mUmCmAmCmGmCfUfGfAmCmAmAmGmCmAmGmA*mG*mA |
| | SEQ ID NO: 298 | mU*fC*mUmCfUmGfCmUmUmGmUmCmAfGmCfGmUmGmAmGmC*mC*mU |
| D:198 | SEQ ID NO: 105 | mA*mG*mAmUmGmGmAmGfGfAfGmGmAmUmAmUmUmCmU*mG*mA |
| | SEQ ID NO: 299 | mU*fC*mAmGfAmAfUmAmUmCmCmUmCfCmUfCmCmAmUmCmU*mG*mA |
| D:199 | SEQ ID NO: 106 | mG*mC*mAmCmAmGmCmAfGfCfAmUmCmGmGmCmUmGmC*mG*mA |
| | SEQ ID NO: 300 | mU*fC*mGmCfAmGfCmCmGmAmUmGmCfUmGfCmUmGmUmGmC*mC*mA |
| D:200 | SEQ ID NO: 107 | mA*mG*mGmAmAmCmAmGfCfCfUmGmGmGmUmCmUmCmU*mA*mA |
| | SEQ ID NO: 301 | mU*fU*mAmGfAmGfAmCmCmCmAmGmGfCmUfGmUmUmCmCmU*mG*mG |
| D:201 | SEQ ID NO: 108 | mG*mG*mAmGmAmUmGmGfUfGfGmCmAmCmAmGmCmAmG*mC*mA |
| | SEQ ID NO: 302 | mU*fG*mCmUfGmCfUmGmUmGmCmCmAfCmCfAmUmCmUmCmC*mC*mG |
| D:202 | SEQ ID NO: 109 | mG*mC*mAmGmCmAmUmCfGfGfCmUmGmCmGmAmCmAmG*mA*mA |
| | SEQ ID NO: 303 | mU*fU*mCmUfGmUfCmGmCmAmGmCmCfGmAfUmGmCmUmGmC*mU*mG |
| D:203 | SEQ ID NO: 110 | mA*mA*mUmUmUmGmAmGfGfUfCmUmUmAmAmAmGmGmC*mU*mA |
| | SEQ ID NO: 304 | mU*fA*mGmCfCmUfUmUmAmAmGmAmCfCmUfCmAmAmAmUmU*mC*mU |
| D:204 | SEQ ID NO: 111 | mA*mA*mGmUmCmCmAmGfCfUfGmAmGmGmAmGmCmGmC*mC*mA |
| | SEQ ID NO: 305 | mU*fG*mGmCfGmCfUmCmCmUmCmAmGfCmUfGmGmAmCmUmU*mC*mU |
| D:205 | SEQ ID NO: 112 | mG*mA*mGmGmUmCmUmUfAfAfAmGmGmCmUmCmAmCmG*mC*mA |
| | SEQ ID NO: 306 | mU*fG*mCmGfUmGfAmGmCmCmUmUmUfAmAfGmAmCmCmUmC*mA*mA |

m indicates 2′O-methyl modified ribose on the listed nucleotide
f indicates 2′F modified ribose on the listed nucleotide
* indicates a phosphorothioate bond (in place of a phosphodiester bond)

TABLE 4
Sense and antisense strands for the GalNAc-ANGPTL8

| | | | | | Start Position |
|---|---|---|---|---|---|
| | | | | | on human Ang8 |
| Duplex | | | | | transcript |
| NO: | Seq ID NO: | Sense21Mer | SEQ ID NO: | AntiSense23Mer | NM_018687.7 |
| D:206 | SEQ ID NO: 307 | UCGGGCAAGCCUGUUGGAGAA | SEQ ID NO: 409 | UUCUCCAACAGGCUUGCCCGAAG | 294 |
| D:207 | SEQ ID NO: 308 | AAUCUGCCUGGAUGGAACUGA | SEQ ID NO: 410 | UCAGUUCCAUCCAGGCAGAUUCA | 618 |
| D:208 | SEQ ID NO: 309 | CCUGGAUGGAACUGAGGACCA | SEQ ID NO: 411 | UGGUCCUCAGUUCCAUCCAGGCA | 624 |
| D:209 | SEQ ID NO: 310 | UUAAAGCAGAGUCGUGGCAUA | SEQ ID NO: 412 | UAUGCCACGACUCUGCUUUAAUG | 851 |
| D:210 | SEQ ID NO: 311 | UGCGACAGAUCCAGGAGAGAA | SEQ ID NO: 413 | UUCUCUCCUGGAUCUGUCGCAGC | 572 |
| D:211 | SEQ ID NO: 312 | UGAAUCUGCCUGGAUGGAACA | SEQ ID NO: 414 | UGUUCCAUCCAGGCAGAUUCAGG | 616 |
| D:212 | SEQ ID NO: 313 | GAGCUGCCUGUUCACUGGGAA | SEQ ID NO: 415 | UUCCCAGUGAACAGGCAGCUCCU | 696 |
| D:213 | SEQ ID NO: 314 | AGGUCUUAAAGGCUCACGCUA | SEQ ID NO: 416 | UAGCGUGAGCCUUUAAGACCUCA | 470 |
| D:214 | SEQ ID NO: 315 | UCACUGGGAUCAGCCAGGGCA | SEQ ID NO: 417 | UGCCCUGGCUGAUCCCAGUGAAC | 707 |
| D:215 | SEQ ID NO: 316 | UCAUUAAAGCAGAGUCGUGGA | SEQ ID NO: 418 | UCCACGACUCUGCUUUAAUGAGG | 848 |
| D:216 | SEQ ID NO: 317 | GAACAGCCUGGGUCUCUAUGA | SEQ ID NO: 419 | UCAUAGAGACCCAGGCUGUUCCU | 213 |
| D:217 | SEQ ID NO: 318 | GGGGCAGGAGGUCAGCCGGGA | SEQ ID NO: 420 | UCCCGGCUGACCUCCUGCCCCAG | 252 |
| D:218 | SEQ ID NO: 319 | AGCGGCUAGAAGUCCAGCUGA | SEQ ID NO: 421 | UCAGCUGGACUUCUAGCCGCUGC | 413 |
| D:219 | SEQ ID NO: 320 | GAGAUGGUGGCACAGCAGCAA | SEQ ID NO: 422 | UUGCUGCUGUGCCACCAUCUCCC | 547 |
| D:220 | SEQ ID NO: 321 | GGCUGACAAAGGCCAGGAACA | SEQ ID NO: 423 | UGUUCCUGGCCUUUGUCAGCCGU | 197 |
| D:221 | SEQ ID NO: 322 | GUCCAGCUGAGGAGCGCCUGA | SEQ ID NO: 424 | UCAGGCGCUCCUCAGCUGGACUU | 424 |
| D:222 | SEQ ID NO: 323 | CAUUAAAGCAGAGUCGUGGCA | SEQ ID NO: 425 | UGCCACGACUCUGCUUUAAUGAG | 849 |
| D:223 | SEQ ID NO: 324 | UCCCAGCCUGAAUCUGCCUGA | SEQ ID NO: 426 | UCAGGCAGAUUCAGGCUGGGAGC | 608 |
| D:224 | SEQ ID NO: 325 | GGAUGCAGCCCAGGAACUUCA | SEQ ID NO: 427 | UGAAGUUCCUGGGCUGCAUCCCG | 276 |
| D:225 | SEQ ID NO: 326 | GCUAGAAGUCCAGCUGAGGAA | SEQ ID NO: 428 | UUCCUCAGCUGGACUUCUAGCCG | 417 |
| D:226 | SEQ ID NO: 327 | CACAGCAGCAUCGGCUGCGAA | SEQ ID NO: 429 | UUCGCAGCCGAUGCUGCUGUGCC | 557 |
| D:227 | SEQ ID NO: 328 | GGAACUUCGGGCAAGCCUGUA | SEQ ID NO: 430 | UACAGGCUUGCCCGAAGUUCCUG | 288 |
| D:228 | SEQ ID NO: 329 | ACUGGGAUCAGCCAGGGCGCA | SEQ ID NO: 431 | UGCGCCCUGGCUGAUCCCAGUGA | 709 |
| D:229 | SEQ ID NO: 330 | AGAAGUCCAGCUGAGGAGCGA | SEQ ID NO: 432 | UCGCUCCUCAGCUGGACUUCUAG | 420 |
| D:230 | SEQ ID NO: 331 | GCAUCGGCUGCGACAGAUCCA | SEQ ID NO: 433 | UGGAUCUGUCGCAGCCGAUGCUG | 564 |
| D:231 | SEQ ID NO: 332 | AGAUGGUGGCACAGCAGCAUA | SEQ ID NO: 434 | UAUGCUGCUGUGCCACCAUCUCC | 548 |
| D:232 | SEQ ID NO: 333 | GGUGGCACAGCAGCAUCGGCA | SEQ ID NO: 435 | UGCCGAUGCUGCUGUGCCACCAU | 552 |
| D:233 | SEQ ID NO: 334 | ACAGCAGCAUCGGCUGCGACA | SEQ ID NO: 436 | UGUCGCAGCCGAUGCUGCUGUGC | 558 |
| D:234 | SEQ ID NO: 335 | AAGGCUCACGCUGACAAGCAA | SEQ ID NO: 437 | UUGCUUGUCAGCGUGAGCCUUUA | 478 |
| D:235 | SEQ ID NO: 336 | AGCCUGAAUCUGCCUGGAUGA | SEQ ID NO: 438 | UCAUCCAGGCAGAUUCAGGCUGG | 612 |
| D:236 | SEQ ID NO: 337 | AUCGGCUGCGACAGAUCCAGA | SEQ ID NO: 439 | UCUGGAUCUGUCGCAGCCGAUGC | 566 |
| D:237 | SEQ ID NO: 338 | AGUCCAGCUGAGGAGCGCCUA | SEQ ID NO: 440 | UAGGCGCUCCUCAGCUGGACUUC | 423 |
| D:238 | SEQ ID NO: 339 | CGGCAGAGGCGGGAGAUGGUA | SEQ ID NO: 441 | UACCAUCUCCCGCCUCUGCCGCU | 535 |
| D:239 | SEQ ID NO: 340 | AUGCUGCAAGGAACACUUCCA | SEQ ID NO: 442 | UGGAAGUGUUCCUUGCAGCAUGA | 648 |
| D:240 | SEQ ID NO: 341 | CUUCGGGCAAGCCUGUUGGAA | SEQ ID NO: 443 | UUCCAACAGGCUUGCCCGAAGUU | 292 |
| D:241 | SEQ ID NO: 342 | GGAGCUGCCUGUUCACUGGGA | SEQ ID NO: 444 | UCCCAGUGAACAGGCAGCUCCUC | 695 |
| D:242 | SEQ ID NO: 343 | GCCUGGAUGGAACUGAGGACA | SEQ ID NO: 445 | UGUCCUCAGUUCCAUCCAGGCAG | 623 |
| D:243 | SEQ ID NO: 344 | CAAGGAACACUUCCACGCCCA | SEQ ID NO: 446 | UGGGCGUGGAAGUGUUCCUUGCA | 654 |
| D:244 | SEQ ID NO: 345 | GGCUAGAAGUCCAGCUGAGGA | SEQ ID NO: 447 | UCCUCAGCUGGACUUCUAGCCGC | 416 |
| D:245 | SEQ ID NO: 346 | UGAGGUCUUAAAGGCUCACGA | SEQ ID NO: 448 | UCGUGAGCCUUUAAGACCUCAAA | 468 |
| D:246 | SEQ ID NO: 347 | GGGCAAGCCUGUUGGAGACUA | SEQ ID NO: 449 | UAGUCUCCAACAGGCUUGCCCGA | 296 |
| D:247 | SEQ ID NO: 348 | GCGGCUAGAAGUCCAGCUGAA | SEQ ID NO: 450 | UUCAGCUGGACUUCUAGCCGCUG | 414 |
| D:248 | SEQ ID NO: 349 | UGGCACAGCAGCAUCGGCUGA | SEQ ID NO: 451 | UCAGCCGAUGCUGCUGUGCCACC | 554 |
| D:249 | SEQ ID NO: 350 | AUGGUGGCACAGCAGCAUCGA | SEQ ID NO: 452 | UCGAUGCUGCUGUGCCACCAUCU | 550 |
| D:250 | SEQ ID NO: 351 | AGAAUUUGAGGUCUUAAAGGA | SEQ ID NO: 453 | UCCUUUAAGACCUCAAAUUCUCG | 462 |
| D:251 | SEQ ID NO: 352 | GGAACAGCCUGGGUCUCUAUA | SEQ ID NO: 454 | UAUAGAGACCCAGGCUGUUCCUG | 212 |
| D:252 | SEQ ID NO: 353 | AGGCUCACGCUGACAAGCAGA | SEQ ID NO: 455 | UCUGCUUGUCAGCGUGAGCCUUU | 479 |
| D:253 | SEQ ID NO: 354 | GGCACAGCAGCAUCGGCUGCA | SEQ ID NO: 456 | UGCAGCCGAUGCUGCUGUGCCAC | 555 |
| D:254 | SEQ ID NO: 355 | CGGGCAAGCCUGUUGGAGACA | SEQ ID NO: 457 | UGUCUCCAACAGGCUUGCCCGAA | 295 |
| D:255 | SEQ ID NO: 356 | AAAGGCUCACGCUGACAAGCA | SEQ ID NO: 458 | UGCUUGUCAGCGUGAGCCUUUAA | 477 |
| D:256 | SEQ ID NO: 357 | GAAUCUGCCUGGAUGGAACUA | SEQ ID NO: 459 | UAGUUCCAUCCAGGCAGAUUCAG | 617 |
| D:257 | SEQ ID NO: 358 | CCUGUUCACUGGGAUCAGCCA | SEQ ID NO: 460 | UGGCUGAUCCCAGUGAACAGGCA | 702 |
| D:258 | SEQ ID NO: 359 | AUUAAAGCAGAGUCGUGGCAA | SEQ ID NO: 461 | UUGCCACGACUCUGCUUUAAUGA | 850 |
| D:259 | SEQ ID NO: 360 | UGGUGGCACAGCAGCAUCGGA | SEQ ID NO: 462 | UCCGAUGCUGCUGUGCCACCAUC | 551 |
| D:260 | SEQ ID NO: 361 | AGGAGGAUAUUCUGCAGCUGA | SEQ ID NO: 463 | UCAGCUGCAGAAUAUCCUCCUCC | 323 |
| D:261 | SEQ ID NO: 362 | UUGAGGUCUUAAAGGCUCACA | SEQ ID NO: 464 | UGUGAGCCUUUAAGACCUCAAAU | 467 |
| D:262 | SEQ ID NO: 363 | GCCCAGGAACUUCGGGCAAGA | SEQ ID NO: 465 | UCUUGCCCGAAGUUCCUGGGCUG | 283 |
| D:263 | SEQ ID NO: 364 | UAGAAGUCCAGCUGAGGAGCA | SEQ ID NO: 466 | UGCUCCUCAGCUGGACUUCUAGC | 419 |
| D:264 | SEQ ID NO: 365 | CUGCCUGUUCACUGGGAUCAA | SEQ ID NO: 467 | UUGAUCCCAGUGAACAGGCAGCU | 699 |
| D:265 | SEQ ID NO: 366 | UCUGCCUGGAUGGAACUGAGA | SEQ ID NO: 468 | UCUCAGUUCCAUCCAGGCAGAUU | 620 |
| D:266 | SEQ ID NO: 367 | CACGCUGACAAGCAGAGCCAA | SEQ ID NO: 469 | UUGGCUCUGCUUGUCAGCGUGAG | 484 |
| D:267 | SEQ ID NO: 368 | UGCCUGGAUGGAACUGAGGAA | SEQ ID NO: 470 | UUCCUCAGUUCCAUCCAGGCAGA | 622 |
| D:268 | SEQ ID NO: 369 | GAUGGUGGCACAGCAGCAUCA | SEQ ID NO: 471 | UGAUGCUGCUGUGCCACCAUCUC | 549 |
| D:269 | SEQ ID NO: 370 | GUCUUAAAGGCUCACGCUGAA | SEQ ID NO: 472 | UUCAGCGUGAGCCUUUAAGACCU | 472 |
| D:270 | SEQ ID NO: 371 | GAUGCAGCCCAGGAACUUCGA | SEQ ID NO: 473 | UCGAAGUUCCUGGGCUGCAUCCC | 277 |
| D:271 | SEQ ID NO: 372 | GAGAAUUUGAGGUCUUAAAGA | SEQ ID NO: 474 | UCUUUAAGACCUCAAAUUCUCGG | 461 |
| D:272 | SEQ ID NO: 373 | GAUGGAGGAGGAUAUUCUGCA | SEQ ID NO: 475 | UGCAGAAUAUCCUCCUCCAUCUG | 318 |
| D:273 | SEQ ID NO: 374 | UGACAAAGGCCAGGAACAGCA | SEQ ID NO: 476 | UGCUGUUCCUGGCCUUUGUCAGC | 200 |
| D:274 | SEQ ID NO: 375 | CUCACGCUGACAAGCAGAGCA | SEQ ID NO: 477 | UGCUCUGCUUGUCAGCGUGAGCC | 482 |
| D:275 | SEQ ID NO: 376 | GCCUGAAUCUGCCUGGAUGGA | SEQ ID NO: 478 | UCCAUCCAGGCAGAUUCAGGCUG | 613 |
| D:276 | SEQ ID NO: 377 | ACAGCCUGGGUCUCUAUGGCA | SEQ ID NO: 479 | UGCCAUAGAGACCCAGGCUGUUC | 215 |
| D:277 | SEQ ID NO: 378 | CAGGAACUUCGGGCAAGCCUA | SEQ ID NO: 480 | UAGGCUUGCCCGAAGUUCCUGGG | 286 |
| D:278 | SEQ ID NO: 379 | GCAAGGAACACUUCCACGCCA | SEQ ID NO: 481 | UGGCGUGGAAGUGUUCCUUGCAG | 653 |
| D:279 | SEQ ID NO: 380 | UUUGAGGUCUUAAAGGCUCAA | SEQ ID NO: 482 | UUGAGCCUUUAAGACCUCAAAUU | 466 |
| D:280 | SEQ ID NO: 381 | GGCCCAGGCACAGAAGGUGCA | SEQ ID NO: 483 | UGCACCUUCUGUGCCUGGGCCAC | 378 |
| D:281 | SEQ ID NO: 382 | UCAGAUGGAGGAGGAUAUUCA | SEQ ID NO: 484 | UGAAUAUCCUCCUCCAUCUGAGU | 315 |
| D:282 | SEQ ID NO: 383 | CCAGGAACUUCGGGCAAGCCA | SEQ ID NO: 485 | UGGCUUGCCCGAAGUUCCUGGGC | 285 |
| D:283 | SEQ ID NO: 384 | CAGCCCAGGAACUUCGGGCAA | SEQ ID NO: 486 | UUGCCCGAAGUUCCUGGGCUGCA | 281 |
| D:284 | SEQ ID NO: 385 | CCCAGCCUGAAUCUGCCUGGA | SEQ ID NO: 487 | UCCAGGCAGAUUCAGGCUGGGAG | 609 |
| D:285 | SEQ ID NO: 386 | CCCAGGAACUUCGGGCAAGCA | SEQ ID NO: 488 | UGCUUGCCCGAAGUUCCUGGGCU | 284 |
| D:286 | SEQ ID NO: 387 | CAUCGGCUGCGACAGAUCCAA | SEQ ID NO: 489 | UUGGAUCUGUCGCAGCCGAUGCU | 565 |
| D:287 | SEQ ID NO: 388 | GUGGCACAGCAGCAUCGGCUA | SEQ ID NO: 490 | UAGCCGAUGCUGCUGUGCCACCA | 553 |
| D:288 | SEQ ID NO: 389 | GGCCCUCACAGGCCACGUGCA | SEQ ID NO: 491 | UGCACGUGGCCUGUGAGGGCCCA | 513 |
| D:289 | SEQ ID NO: 390 | AACUUCGGGCAAGCCUGUUGA | SEQ ID NO: 492 | UCAACAGGCUUGCCCGAAGUUCC | 290 |
| D:290 | SEQ ID NO: 391 | UAAAGGCUCACGCUGACAAGA | SEQ ID NO: 493 | UCUUGUCAGCGUGAGCCUUUAAG | 476 |
| D:291 | SEQ ID NO: 392 | UUCGGGCAAGCCUGUUGGAGA | SEQ ID NO: 494 | UCUCCAACAGGCUUGCCCGAAGU | 293 |
| D:292 | SEQ ID NO: 393 | AGCAUCGGCUGCGACAGAUCA | SEQ ID NO: 495 | UGAUCUGUCGCAGCCGAUGCUGC | 563 |
| D:293 | SEQ ID NO: 394 | AUCUGCCUGGAUGGAACUGAA | SEQ ID NO: 496 | UUCAGUUCCAUCCAGGCAGAUUC | 619 |
| D:294 | SEQ ID NO: 395 | GCUCACGCUGACAAGCAGAGA | SEQ ID NO: 497 | UCUCUGCUUGUCAGCGUGAGCCU | 481 |
| D:295 | SEQ ID NO: 396 | AGAUGGAGGAGGAUAUUCUGA | SEQ ID NO: 498 | UCAGAAUAUCCUCCUCCAUCUGA | 317 |
| D:296 | SEQ ID NO: 397 | GCACAGCAGCAUCGGCUGCGA | SEQ ID NO: 499 | UCGCAGCCGAUGCUGCUGUGCCA | 556 |
| D:297 | SEQ ID NO: 398 | AGGAACAGCCUGGGUCUCUAA | SEQ ID NO: 500 | UUAGAGACCCAGGCUGUUCCUGG | 211 |
| D:298 | SEQ ID NO: 399 | GGAGAUGGUGGCACAGCAGCA | SEQ ID NO: 501 | UGCUGCUGUGCCACCAUCUCCCG | 546 |
| D:299 | SEQ ID NO: 400 | GCAGCAUCGGCUGCGACAGAA | SEQ ID NO: 502 | UUCUGUCGCAGCCGAUGCUGCUG | 561 |
| D:300 | SEQ ID NO: 401 | AAUUUGAGGUCUUAAAGGCUA | SEQ ID NO: 503 | UAGCCUUUAAGACCUCAAAUUCU | 464 |
| D:301 | SEQ ID NO: 402 | AAGUCCAGCUGAGGAGCGCCA | SEQ ID NO: 504 | UGGCGCUCCUCAGCUGGACUUCU | 422 |
| D:302 | SEQ ID NO: 403 | GAGGUCUUAAAGGCUCACGCA | SEQ ID NO: 505 | UGCGUGAGCCUUUAAGACCUCAA | 469 |
| D:303 | SEQ ID NO: 404 | AGCAGCAUCGGCUGCGACAGA | SEQ ID NO: 506 | UCUGUCGCAGCCGAUGCUGCUGU | 560 |
| D:304 | SEQ ID NO: 405 | GGAGCUGACCCUGCUCUUCCA | SEQ ID NO: 507 | UGGAAGAGCAGGGUCAGCUCCUC | 120 |
| D:305 | SEQ ID NO: 406 | GACUCAGAUGGAGGAGGAUAU | SEQ ID NO: 508 | AUAUCCUCCUCCAUCUGAGUCUC | 312 |
| D:306 | SEQ ID NO: 407 | CUUAAAGGCUCACGCUGACAA | SEQ ID NO: 509 | UUGUCAGCGUGAGCCUUUAAGAC | 474 |
| D:307 | SEQ ID NO: 408 | AAGCCUGUUGGAGACUCAGAU | SEQ ID NO: 510 | AUCUGAGUCUCCAACAGGCUUGC | 300 |

### Human ANGPTL8 Transcript NM 018687.7 SEQ ID 511:

ataccttaga ccctcagtca tgccagtgcc tgctctgtgc ctgctctggg ccctggcaat ggtgacccgg cctgcctcag
cggcccccat gggcggccca gaactggcac agcatgagga gctgaccctg ctcttccatg ggaccctgca gctgggccag
gccctcaacg gtgtgtacag gaccacggag ggacggctga caaaggccag gaacagectg ggtctctatg gccgcacaat
agaactcctg gggcaggagg tcagccgggg ccgggatgca gcccaggaac ttcgggcaag cctgttggag actcagatgg
aggaggatat tctgcagctg caggcagagg ccacagctga ggtgctgggg gaggtggccc aggcacagaa ggtgctacgg
gacagcgtgc agcggctaga agtccagctg aggagcgcct ggctgggccc tgcctaccga gaatttgagg tcttaaaggc
tcacgctgac aagcagagcc acatcctatg ggccctcaca ggccacgtgc agcggcagag gcgggagatg gtggcacagc
agcatcggct gcgacagatc caggagagac tccacacagc ggcgctocca gcctgaatct gcctggatgg aactgaggac
caatcatgct gcaaggaaca cttccacgcc ccgtgaggcc cctgtgcagg gaggagctgc ctgttcactg ggatcagcca
gggcgccggg ccccacttct gagcacagag cagagacaga cgcaggcggg gacaaaggca gaggatgtag ccccattggg
gaggggtgga ggaaggacat gtaccctttc atgcctacac acccctcatt aaagcagagt cgtggcatct ca

### Example 4

### In Vitro Knockdown of Human ANGPTL8 in AAV-ANGPTL8 Humanized Mouse Primary Hepatocytes (MPH) with GalNAc-Conjugated ANGPTL8 siRNA

Knockdown of ANGPTL8 expression by the LYGal1-conjugated ANGPTL8 siRNA was assayed using the following procedure: mouse primary hepatocytes (MPH) were freshly isolated from an AAV-ANGPTL8 humanized mouse, added to Corning 96-well plates at 15,000 cells per well, and siRNA were added directly to the well. For single point (SP) screening, 1 uM (1,000 nM) of GalNAc-conjugated siRNA was used. To generate concentration/dose response curves final concentrations of 1000, 333, 111, 37, 12, 4, 1.37, 0.46, 0.15, 0.05, and 0.017 nM of GalNAc-conjugated siRNA concentration were used. Treated cells were lysed and RNA was isolated using the Quick-RNA 96 Kit (Zymo Research) directly into the 96 well plate. The eluted RNA was used immediately or stored frozen. cDNA was synthesized using Fast Advanced RT Master Mix (Invitrogen) and using the following steps in a thermocycler: 37° C. for 30 minutes, 95 C for 5 minutes, and 4 C hold. Polymerase Chain Reaction (PCR) was performed via TaqMan RT PCR (Life Technologies) using the following cycles temperatures and times: 50° C. for 2 minutes, 95° C. for 10 minutes, 40 cycles of 95° C. for 15 seconds and 60° C. for 1 minute.

The human ANGPTL8 levels were normalized to mouse Rp1p0 (Life Technologies) and represent the relative knockdown of human ANGPTL8 mRNA expression as compared to vehicle-treated control cells. IC₅₀ values were calculated using a 4-parameter fit model using XLFit.

Table 5 shows the result of a single dose screen in AAV-ANGPTL8 humanized mouse primary hepatocytes by free uptake with the indicated GalNAc-conjugated ANGPTL8 siRNA. Data were expressed as percent of message knockdown relative to untreated cells. The IC₅₀ and percent maximum knockdown of top hits from single point screening followed by concentration/dose response curves are included as well.

TABLE 5
Percent knockdown of single dose screen and IC₅₀ with percent
maximum knockdown of top hits from single point screen
in AAV-ANGPTL8 humanized mouse primary hepatocytes

| Duplex No: | % KD @ 1 uM | % KD @ 1 uM | IC50 (nM) | Max % KD |
|---|---|---|---|---|
| D: 70 | 94.95 | | 13.04 | 90.99 |
| D: 72 | 94.67 | | 9.27 | 88.82 |
| D: 82 | 94.20 | | 2.01 | 95.81 |
| D: 6 | 93.31 | 91.33 | 4.69 | 93.41 |
| D: 87 | 93.04 | | 28.29 | 71.41 |
| D: 93 | 92.40 | | 10.64 | 85.63 |
| D: 34 | 89.56 | 88.55 | 13.83 | 75.12 |
| D: 92 | 89.39 | | 19.9 | 82.05 |
| D: 51 | 89.13 | 88.34 | 16.1 | 83.12 |
| D: 61 | 87.06 | 82.43 | | |
| D: 73 | 86.51 | | 11.09 | 77.68 |
| D: 19 | 85.54 | 78.46 | | |
| D: 80 | 84.53 | | | |
| D: 36 | 82.20 | 76.47 | 8.51 | 72.37 |
| D: 46 | 81.11 | 77.01 | 24.79 | 79.52 |
| D: 63 | 78.01 | | | |
| D: 86 | 77.65 | | | |
| D: 10 | 77.36 | 69.09 | | |
| D: 47 | 76.94 | 78.71 | | |
| D: 101 | 76.48 | | | |
| D: 98 | 76.16 | | | |
| D: 48 | 74.82 | 72.20 | | |
| D: 4 | 73.16 | 67.97 | | |
| D: 103 | 70.74 | | | |
| D: 90 | 70.67 | | | |
| D: 78 | 70.61 | | | |
| D: 62 | 67.97 | | | |
| D: 64 | 67.48 | | | |
| D: 31 | 67.29 | 68.33 | | |
| D: 67 | 65.86 | | | |
| D: 13 | 64.91 | 50.26 | | |
| D: 27 | 62.73 | 42.21 | | |
| D: 18 | 61.81 | 46.20 | | |
| D: 56 | 59.45 | 63.92 | | |
| D: 26 | 56.02 | 45.27 | | |
| D: 45 | 53.90 | 33.84 | | |
| D: 55 | 52.79 | 47.87 | | |
| D: 24 | 52.33 | 55.12 | | |
| D: 99 | 52.07 | | | |
| D: 30 | 48.47 | 40.63 | | |
| D: 17 | 48.10 | 35.59 | | |
| D: 96 | 47.90 | | | |
| D: 60 | 46.75 | 39.77 | | |
| D: 88 | 46.59 | | | |
| D: 75 | 45.70 | | | |
| D: 20 | 45.58 | 12.43 | | |
| D: 91 | 42.67 | | | |
| D: 52 | 39.83 | 17.25 | | |
| D: 25 | 39.40 | 28.67 | | |
| D: 77 | 39.06 | | | |
| D: 74 | 38.73 | | | |
| D: 89 | 38.09 | | | |
| D: 38 | 36.07 | 11.81 | | |
| D: 68 | 35.74 | | | |
| D: 84 | 35.73 | | | |
| D: 97 | 32.66 | | | |
| D: 29 | 32.63 | 6.98 | | |
| D: 49 | 32.35 | 29.83 | | |
| D: 59 | 30.09 | −3.84 | | |
| D: 50 | 29.67 | 15.48 | | |
| D: 69 | 29.26 | | | |
| D: 53 | 28.66 | 22.18 | | |
| D: 33 | 28.15 | 20.07 | | |
| D: 81 | 26.88 | | | |
| D: 95 | 25.86 | | | |
| D: 65 | 24.04 | | | |
| D: 28 | 23.21 | −4.56 | | |
| D: 71 | 23.01 | | | |
| D: 37 | 22.98 | −4.90 | | |
| D: 41 | 22.70 | −11.81 | | |
| D: 32 | 21.77 | 2.55 | | |
| D: 16 | 20.17 | 18.15 | | |
| D: 100 | 19.82 | | | |
| D: 57 | 19.49 | 4.87 | | |
| D: 21 | 19.30 | −25.71 | | |
| D: 66 | 16.48 | | | |
| D: 102 | 16.31 | | | |
| D: 43 | 16.21 | −12.66 | | |
| D: 40 | 15.69 | 8.32 | | |
| D: 14 | 14.28 | −15.81 | | |
| D: 42 | 14.13 | −3.35 | | |
| D: 83 | 13.98 | | | |
| D: 35 | 13.92 | −6.21 | | |
| D: 54 | 13.10 | −4.76 | | |
| D: 15 | 11.10 | −0.72 | | |
| D: 22 | 10.22 | −17.00 | | |
| D: 76 | 10.21 | | | |
| D: 11 | 6.67 | −8.58 | | |
| D: 12 | 4.20 | −28.78 | | |
| D: 58 | 4.00 | −1.18 | | |
| D: 8 | 3.56 | −14.25 | | |
| D: 23 | 0.43 | −32.92 | | |
| D: 85 | −1.01 | | | |
| D: 94 | −3.35 | | | |
| D: 79 | −6.23 | | | |
| D: 9 | −9.07 | −34.19 | | |
| D: 7 | −16.36 | −17.82 | | |
| D: 39 | −37.59 | −44.25 | | |

### Example 5

### In Vitro Knockdown of Human ANGPTL8 in Hep3B Cells with GalNAc-Conjugated ANGPTL8 siRNA

Knockdown of ANGPTL8 expression by the LYGal1-conjugated ANGPTL8 siRNA was assayed using the following procedure: transfection reagent RNAiMAX (Life Technologies) at 0.3 μl/well was mixed with siRNA in Corning 96-well plates before adding Hep3B cells (ATCC) at 8,000 cells per well. To generate concentration/dose response curves final concentrations of 100, 33.3, 11.1, 3.7, 1.2, 0.4, 0.137, 0.046, 0.015, 0.005, and 0.0017 nM of GalNAc-conjugated siRNA concentration was used.

Treated cells are lysed and RNA was isolated using the Quick-RNA 96 Kit (Zymo Research) directly into the 96 well plate. The eluted RNA was used immediately or stored frozen. cDNA was synthesized using Fast Advanced RT Master Mix (Invitrogen) and using the following steps in a thermocycler: 37° C. for 30 minutes, 95° C. for 5 minutes, and 4° C. hold. Polymerase Chain Reaction (PCR) was performed via TaqMan RT PCR (Life Technologies) using the following cycles temperatures and times: 50° C. for 2 minutes, 95° C. for 10 minutes, 40 cycles of 95° C. for 15 seconds and 60° C. for 1 minute.

The human ANGPTL8 levels were normalized to human RPLP0 (Life Technologies) and represent the relative knockdown of human ANGPTL8 mRNA expression as compared to vehicle-treated control cells. IC₅₀ values were calculated using a 4-parameter fit model using XLFit.

Table 6 shows the result of IC₅₀ and percent maximum knockdown calculated from concentration/dose response curves in Hep3B cells by transfection with the indicated ANGPTL8 siRNA. In most instances, duplexes resulted in over 90% knockdown, with nanomolar or lower IC₅₀.

TABLE 6
IC₅₀ and percent maximum knockdown of ANGPTL8
message in Hep3B cells by transfection reagent,
RNAiMAX, with the indicated ANGPTL8 siRNA

| Duplex No: | IC50 (nM) | % KD (10 nM) |
|---|---|---|
| D: 172 | 1.140 | 98.73 |
| D: 174 | 0.900 | 90.10 |
| D: 184 | 0.680 | 89.97 |
| D: 109 | 2.070 | 79.44 |
| D: 87 | 0.125 | 98.08 |
| D: 93 | 0.049 | 98.86 |
| D: 195 | 1.400 | 91.33 |
| D: 137 | 2.090 | 100.00 |
| D: 194 | 3.220 | 85.71 |
| D: 153 | 0.950 | 92.82 |
| D: 175 | 3.950 | 66.22 |
| D: 36 | 0.079 | 77.97 |
| D: 148 | 3.110 | 83.93 |
| D: 10 | 0.049 | 94.40 |
| D: 30 | 0.166 | 82.67 |
| D: 68 | 0.034 | 100.00 |
| D: 59 | 0.019 | 98.50 |
| D: 33 | 0.050 | 25.59 |
| D: 65 | 0.101 | 96.12 |
| D: 28 | 0.351 | 29.82 |
| D: 41 | 0.047 | 79.42 |
| D: 100 | 0.033 | 97.47 |
| D: 57 | 0.294 | 94.35 |
| D: 66 | 0.033 | 98.11 |
| D: 43 | 0.144 | 99.37 |
| D: 14 | 0.127 | 79.61 |
| D: 42 | 0.079 | 100.00 |
| D: 35 | 0.113 | 94.88 |
| D: 15 | 0.010 | 94.33 |
| D: 22 | 0.005 | 96.46 |
| D: 76 | 0.328 | 95.66 |
| D: 11 | 0.215 | 98.70 |
| D: 12 | 0.157 | 96.37 |
| D: 58 | 0.081 | 99.78 |
| D: 8 | 0.053 | 89.40 |
| D: 23 | 0.185 | 97.81 |
| D: 85 | 0.180 | 99.86 |
| D: 94 | 0.020 | 99.46 |
| D: 79 | 0.058 | 100.00 |
| D: 9 | 0.026 | 94.52 |
| D: 7 | 0.104 | 100.00 |
| D: 39 | 0.042 | 98.02 |
| D: 105 | 0.285/2.47 | 77.61/53.92 |

### Example 6: In Vivo Single Dose Screen

GalNAc-siRNA (n=16) were tested in male C57bl/6 mice (Taconic farms). The siRNAs were tested in a single study. Mice were dosed by retro-orbital injection with an adeno-associated virus (AAV) vector containing a plasmid with an albumin promoter and the coding sequence for human ANGPTL8 (NM_018687.7) (Vector BioLabs). Blood was collected from mice via retro-orbital sinus 14 days post AAV administration. Serum was prepared from blood and triglycerides were measured utilizing a COBAS clinical chemistry analyzer (Roche) and ANGPTL4/8 was measured by ELISA (Meso Scale Diagnostics). Body weight of mice were measured 22 days after AAV administration. Mice were assigned to groups with similar body weight, serum triglyceride levels, and serum ANGPTL4/8 (n=6/group). Either PBS or GalNac-siRNA test article, at a dose of 5 mg/kg, was administered subcutaneously to mice. Seven days post subcutaneous injection, blood was collected from all mice and serum is analyzed for triglycerides. Fourteen days post subcutaneous injection mice were euthanized under isoflurane anesthesia, blood was collected, and serum was analyzed for triglycerides. Liver was collected from the mice and frozen in liquid nitrogen. Triglyceride as a percent change from time-matched PBS was calculated as ((triglyceride minus triglyceride of PBS group)/(triglyceride of PBS group))*100. Livers were homogenized in TriZol (Invitrogen) using Lysing Matrix D bead tubes on a FastPrep-24 (MP Bio). Chloroform was added and the aqueous phase is mixed with ethanol to precipitate the RNA. RNA was isolated on columns using PureLink Pro96 Total RNA purification kit (Invitrogen) according to manufacturer's protocol and quantified on a NanoDrop (ThermoFisher). Equal amounts (1 ug) of RNA were reverse transcribed to cDNA using High-Capacity cDNA Reverse Transcription kit (Life Technologies) on Mastercycler Nexus (Eppendorf). Thermocycler settings were 25° C. for 10 min, 37° C. for 2 hrs, then 85° C. for 5 min. Template cDNA was combined with Taqman Universal Master Mix and Assays on Demand primer/probesets and RT-PCR was performed on the QuantStudio Pro7 (ThermoFisher) with the following parameters: 50° C. for 2 min, 95° C. for 10 minutes, then 40 cycles of 95° C. for 15 sec and 60° C. for 1 min. Fold changes (FC) were calculated as follows: the CT value of mouse Rp1p0 was subtracted from CT value of human ANGPTL8 to obtain the delta CT value. Then the delta delta CT value was calculated by subtracting the delta CT value of the untreated sample (average of PBS control) from the delta CT value of each test sample. Fold change was calculated by taking the log base 2 of the negative delta delta CT value. Percent mRNA remaining was calculated multiplying the fold change by 100. Data is shown in Table 7.

TABLE 7
C57 mice hANGPTL8 AAV in vivo single dose screen

| | 7 day, 5 mpk, | 2 wk, 5 mpk, | |
|---|---|---|---|
| | Trigs % Change | Trigs % Change | 2 wk, 5 mpk, |
| Duplex No: | from PBS | from PBS | % mRNA KD |
| D: 172 | −26 | −25 | 90.6 |
| D: 174 | −30 | −36 | 86.7 |
| D: 82 | −43 | −48 | 88.4 |
| D: 184 | −42 | −48 | 90.1 |
| D: 6 | −18 | −34 | 87 |
| D: 109 | −21 | −41 | 83.3 |
| D: 189 | −21 | −16 | 49.8 |
| D: 195 | −46 | −50 | 80.9 |
| D: 137 | −24 | −31 | 82.1 |
| D: 194 | −14 | −22 | 84.2 |
| D: 153 | −23 | −19 | 81.7 |
| D: 175 | −29 | −34 | 82 |
| D: 139 | −35 | −34 | 64.8 |
| D: 148 | −5 | −14 | 82.9 |
| D: 2 | −70 | −85 | 79 |
| D: 105 | −70 | −88 | 81 |

### Example 7: In Vivo Durability 8 Week

GalNAc-siRNA (n=11) were tested in male mice transgenic for human cholesterol ester transfer protein (CETP) and human apolipoprotein A1 (Taconic farms). The siRNAs were divided and tested in 2 studies (n=7 and n=4). Mice were dosed by retro-orbital injection with two adeno-associated virus (AAV) vectors. One vector contained a plasmid with an albumin promoter and the coding sequence for human ANGPTL8 (NM_018687.7) (Vector BioLabs). The second vector contained a mouse codon optimized sequence of human ANGPTL3 (NP_055310.1) (Vector BioLabs). Blood was collected from mice 3-4 weeks post AAV administration. This was considered the baseline blood collection. Serum was prepared from blood and ANGPTL3/8 was measured by an ELISA (Meso Scale Diagnostics) and triglycerides were measured as described. Body weight of mice was measured and mice were assigned to groups with similar body weight, serum triglyceride, and ANGPTL3/8 levels (n=9/group). 4-5 weeks post AAV administration, either PBS or test article GalNac-siRNA, at doses 1.75 and 10 mg/kg were administered subcutaneously to mice. At 2 weeks post siRNA administration, 3 mice from each group were euthanized under isoflurane anesthesia, blood was collected, and serum was analyzed for triglycerides. Liver was collected from the mice and frozen in liquid nitrogen. Blood was collected from the remaining mice (n=6/group) at 2, 4, and 6 weeks post siRNA administration under isoflurane anesthesia. Serum was prepared from blood and triglycerides are measured. At 8 weeks post siRNA administration, the remaining mice were euthanized under isoflurane anesthesia. Blood and liver were collected from mice. Livers were processed and mRNA remaining was calculated as described in the in vivo single dose screen. Triglyceride as a percent change from PBS is calculated as described in the in vivo single dose screen.

TABLE 8
In vivo Durability 8 week Dose Response (C57 mice hANGPTL8 AAV)

| | 2 Weeks | | | | 3 Weeks | | | 4 Weeks | |
|---|---|---|---|---|---|---|---|---|---|
| | 1.75 | 10 | | | 0.3 | 1.75 | 10 | 1.75 | 10 |
| | mpk, | mpk, | | | mpk, | mpk, | mpk, | mpk, | mpk, |
| | Trigs % | Trigs % | 1.75 | 10 | Trigs % | Trigs % | Trigs % | Trigs % | Trigs % |
| | Change | Change | mpk, | mpk, | Change | Change | Change | Change | Change |
| Duplex | from | from | % mRNA | % mRNA | from | from | from | from | from |
| No: | PBS | PBS | remaining | remaining | PBS | PBS | PBS | PBS | PBS |
| D: 172 | −55 | −79 | 26 | 9 | | | | −54 | −70 |
| D: 174 | −71 | −82 | 20 | 9 | | | | −65 | −80 |
| D: 184 | −74 | −85 | 19 | 7 | | | | −65 | −78 |
| D: 109 | −46 | −71 | 24 | 9 | | | | −47 | −76 |
| D: 195 | −67 | −77 | 16 | 5 | | | | −69 | −83 |
| D: 137 | −69 | −89 | 25 | 7 | | | | −54 | −79 |
| D: 194 | −57 | −71 | 27 | 10 | | | | −45 | −69 |
| D: 153 | −51 | −71 | 38 | 12 | | | | −53 | −62 |
| D: 175 | −59 | −76 | 34 | 13 | | | | −52 | −67 |
| D: 148 | −56 | −75 | 19 | 7 | | | | −39 | −65 |
| D: 4 | | | | | −9 | −27 | −47 | | |
| D: 1 | | | | | −15 | −38 | −73 | | |
| D: 2 | | | | | −28 | −78 | −94 | | |
| D: 105 | −84 | −97 | 25 | 5 | | | | −72 | −94 |
| D: 3 | | | | | −20 | −53 | −80 | | |
| D: 5 | | | | | −20 | −41 | −65 | | |

| | 6 Weeks | | | 8 Weeks | | | |
|---|---|---|---|---|---|---|---|
| | 0.3 | 1.75 | 10 | 1.75 | 10 | | |
| | mpk, | mpk, | mpk, | mpk, | mpk, | | |
| | Trigs % | Trigs % | Trigs % | Trigs % | Trigs % | 1.75 | 10 |
| | Change | Change | Change | Change | Change | mpk, | mpk, |
| Duplex | from | from | from | from | from | % mRNA | % mRNA |
| No: | PBS | PBS | PBS | PBS | PBS | remaining | remaining |
| D: 172 | | −50 | −72 | −40 | −73 | 42 | 14 |
| D: 174 | | −54 | −72 | −54 | −66 | 29 | 23 |
| D: 184 | | −64 | −80 | −51 | −76 | 43 | 18 |
| D: 109 | | −43 | −63 | −46 | −64 | 47 | 16 |
| D: 195 | | −60 | −83 | −54 | −77 | 33 | 7 |
| D: 137 | | −46 | −73 | −35 | −69 | 64 | 23 |
| D: 194 | | −48 | −66 | −33 | −68 | 47 | 18 |
| D: 153 | | −44 | −62 | −50 | −68 | 42 | 22 |
| D: 175 | | −47 | −67 | −37 | −67 | 47 | 22 |
| D: 148 | | −44 | −67 | −33 | −65 | 43 | 18 |
| D: 4 | −12 | −22 | −39 | | | | |
| D: 1 | −12 | −28 | −53 | | | | |
| D: 2 | −21 | −60 | −90 | | | | |
| D: 105 | | −60 | −92 | −55 | −87 | 40 | 6 |
| D: 3 | −5 | −43 | −71 | | | | |
| D: 5 | −6 | −33 | −63 | | | | |

### Example 8: In Vivo Durability 15 Week (Dose Response (C57 Mice hANGPTL8 AAV))

GalNAc-siRNA (n=5) were tested in male mice transgenic for human cholesterol ester transfer protein (CETP) and human apolipoprotein A1 (Taconic farms). The siRNAs were tested in a single study. Mice were dosed by retro-orbital injection with two adeno-associated virus (AAV) vectors. One vector contained a plasmid with an albumin promoter and the coding sequence for human ANGPTL8(NM_018687.7) (Vector BioLabs). The second vector contained a mouse codon optimized sequence of human ANGPTL3(NP_055310.1) (Vector BioLabs). Blood was collected from mice 3 weeks post AAV administration. This was considered the baseline blood collection. Serum was prepared from blood and triglycerides and ANGPTL3/8 was measured. Body weight of mice was measured and mice were assigned to groups with similar body weight, serum triglyceride and ANGPTL3/8 levels (n=10/group). 4 weeks post AAV administration, either PBS or test article GalNac-siRNA, at doses 0.3, 1.75 and 10 mg/kg were administered subcutaneously to mice. At 2 weeks post siRNA administration, 3 mice from each group were euthanized under isoflurane anesthesia, blood was collected, and serum was analyzed for triglycerides. Liver was collected from the mice and frozen in liquid nitrogen. Blood was collected from the remaining mice (n=7/group) at 3, 6, 9, and 12 weeks post siRNA administration under isoflurane anesthesia. Serum was prepared from blood and triglycerides are measured. At 15 weeks post siRNA administration, the remaining mice were euthanized under isoflurane anesthesia. Blood and liver were collected from mice. Serum was prepared from blood and triglycerides were measured. Livers were processed and mRNA remaining was calculated as described in the in vivo single dose screen. Triglyceride as a percent change from PBS was calculated as described in the in vivo single dose screen.

TABLE 9
Dose Response (C57 mice hANGPTL8 AAV)

| | 3 Weeks | | | 6 Weeks | | | 8 Weeks | | | 9 Weeks | | | 12 Weeks | | |
|---|---|---|---|---|---|---|---|---|---|---|---|---|---|---|---|
| | 0.3 | 1.75 | | 0.3 | 1.75 | 10 | 0.3 | 1.75 | 10 | 0.3 | 1.75 | 10 | 0.3 | 1.75 | 10 |
| | mpk, | mpk, | 10 | mpk, | mpk, | mpk, | mpk, | mpk, | mpk, | mpk, | mpk, | mpk, | mpk, | mpk, | mpk, |
| | Trigs | Trigs | mpk, | Trigs | Trigs | Trigs | Trigs | Trigs | Trigs | Trigs | Trigs | Trigs | Trigs | Trigs | Trigs |
| | % | % | % | % | % | % | % | % | % | % | % | % | % | % | % |
| Du- | Change | Change | mRNA | Change | Change | Change | Change | Change | Change | Change | Change | Change | Change | Change | Change |
| plex | from | from | remain- | from | from | from | from | from | from | from | from | from | from | from | from |
| NO: | PBS | PBS | ing | PBS | PBS | PBS | PBS | PBS | PBS | PBS | PBS | PBS | PBS | PBS | PBS |
| D: 1 | −15 | −38 | −73 | −12 | −28 | −53 | 8 | −19 | −51 | −20 | −23 | −42 | −11 | −9 | −22 |
| D: 2 | −28 | −78 | −94 | −21 | −60 | −90 | 9 | −54 | −82 | −18 | −49 | −79 | 6 | −25 | −68 |
| D: 3 | −20 | −53 | −80 | −5 | −43 | −71 | 4 | −12 | −61 | −18 | −31 | −59 | −8 | 3 | −35 |
| D: 4 | −20 | −41 | −65 | −6 | −33 | −63 | 20 | 1 | −36 | −13 | −24 | −59 | 0 | 9 | −24 |
| D: 5 | −9 | −27 | −47 | −12 | −22 | −39 | 12 | 12 | −42 | −13 | −24 | −31 | 32 | −16 | −12 |

1. An RNAi agent for reducing ANGPTL8 gene expression, wherein the RNAi agent comprises a delivery moiety of Formula I conjugated to R, wherein R is a double stranded RNA (dsRNA) comprising an antisense strand and a sense strand: 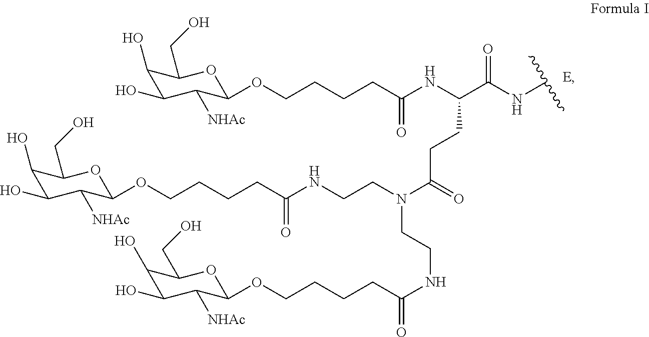
wherein R is conjugated to connection point E of Formula I, optionally via a linker, wherein the sense strand and the antisense strand form a duplex region, and wherein the antisense strand comprises any one of SEQ ID NOs: 6, 7, 8, 9, 10, 11, 12, 13, 14, or 15, or a sequence having 90% sequence identity thereto, or an antisense strand sequence as set forth in Tables 3A, 3B, and 4 or a sequence having 90% sequence identity thereto, and wherein the sense and antisense strand each optionally comprise one or more modified nucleotides and optionally one or more modified internucleotide linkages.

2. The RNAi agent of claim 1, wherein Formula I is conjugated to the sense strand, optionally via a linker.

3. The RNAi agent of claim 2, wherein Formula I is conjugated to the 3′ terminal nucleotide of the sense strand, optionally via a linker.

4. The RNAi agent of claim 1, wherein the sense strand is 15 to 25 nucleotides in length.

5. The RNAi agent of claim 1, wherein the antisense strand is between 18 and 23 nucleotides in length.

6. The RNAi agent of claim 1, wherein the sense strand is between 18 and 21 nucleotides in length.

7. The RNAi agent of claim 1, wherein the antisense strand is 23 nucleotides in length and the sense strand is 21 nucleotides in length.

8. The RNAi agent of claim 1, wherein the sense strand and the antisense strand form a region of complementarity of at least 18 nucleotides in length.

9. The RNAi agent of claim 1, wherein the duplex region between the sense strand and the antisense strand comprises 0, 1, or 2 mismatches between the sense strand and the antisense strand.

10. The RNAi agent of claim 1, wherein the duplex region between the sense strand and the antisense strand comprises 0 mismatches between the sense strand and the antisense strand.

11. The RNAi agent of claim 1, wherein the antisense strand comprises 15 contiguous nucleotides of any one of SEQ ID NOs: 6, 7, 8, 9, 10, 11, 12, 13, 14, or 15, or a sequence of Tables 3A, 3B, and 4.

12. The RNAi agent of claim 1, wherein the antisense strand comprises 18 contiguous nucleotides of any one of SEQ ID NOs: 6, 7, 8, 9, 10, 11, 12, 13, 14, or 15, or a sequence of Tables 3A, 3B, and 4.

13. The RNAi agent of claim 1, wherein the sense strand is selected from the group consisting of SEQ ID NOs: 1 to 5, or a sequence having at least 90% sequence identity thereto, or a sense strand sequence set forth in Table 3, or a sequence having 90% sequence identity thereto.

14. The RNAi agent of claim 1, wherein the sense strand or the antisense strand each independently comprise one or more modified nucleotides.

15. The RNAi agent of claim 1, wherein each nucleotide of the sense strand and each nucleotide of the antisense strand is a modified nucleotide, and the modified nucleotides are independently 2′ fluoro modified nucleotide residues or 2′-O-methyl modified nucleotides.

16. The RNAi agent of claim 1, wherein the antisense strand is 23 nucleotides in length and wherein each nucleotide of the antisense strand is a modified nucleotide, and wherein the position of the 2′ fluoro modified nucleotides is modified to be present at a group of positions selected from the following:
a. Positions 2, 3, 7, 14, and 16 from the 5′ end of the antisense strand; or
b. Positions 2, 5, 7, 14, and 16 from the 5′ end of the antisense strand; or
c. Positions 2, 3, 8, 14, and 16 from the 5′ end of the antisense strand; or
d. Positions 2, 5, 8, 14, and 16 from the 5′ end of the antisense strand; or
e. Positions 2, 6, 14, and 16 from the 5′ end of the antisense strand.

17. The RNAi agent of claim 1, wherein the sense strand and antisense strand each independently comprise one or more modified internucleotide linkages, and wherein each modified internucleotide linkage is a phosphorothioate linkage.

18. The RNAi agent of claim 1, wherein the sense strand and antisense strand each independently comprise four phosphorothioate linkages.

19. The RNAi agent of claim 1, wherein the 5′ terminal nucleotide of the antisense strand comprises an OH group, a phosphate group, a vinyl phosphonate, or a phosphate analog.

20. The RNAi agent of claim 19, wherein the 5′ terminal nucleotide of the antisense strand is further modified to replace the 5′ phosphate group with an OH group.

21. The RNAi agent of claim 1, wherein R is conjugated to Formula I via a linker.

22. The RNAi agent of claim 1, wherein R is conjugated to Formula I via a linker, and wherein linker comprises a linker of Formula II having connection points A and B or the linker comprises Formula III having connection points C and D, and wherein: 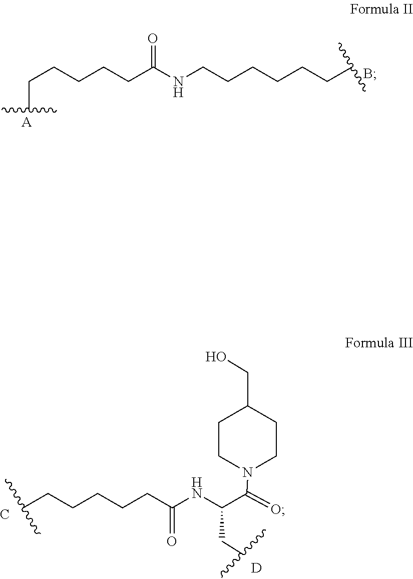
a. Formula I, at connection point E, is conjugated to Formula II at connection point A and Formula II is conjugated to a phosphate group or a phosphorothioate group at connection point B, and the phosphate group or phosphorothioate group is further conjugated to R; or
b. Formula I, at connection point E, is conjugated to Formula III at connection point C and Formula III is conjugated to a phosphate group or phosphorothioate group at connection point D, and the phosphate group or phosphorothioate group is further conjugated to R.

23. The RNAi agent of claim 1, wherein R is conjugated to Formula I via a linker, and wherein the linker is a linker comprising Formula III having connection points C and D: 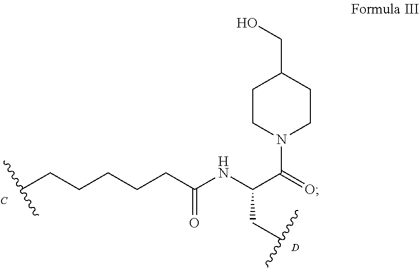
and wherein Formula I, at connection point E, is conjugated to Formula III at connection point C and Formula III is conjugated to a phosphate group or a phosphorothioate group at connection point D, and the phosphate group or the phosphorothioate group is further conjugated to R.

24. (canceled)

25. (canceled)

26. (canceled)

27. (canceled)

28. A pharmaceutical composition comprising the RNAi agent of claim 1, and one or more pharmaceutically acceptable excipients.

29. (canceled)

30. (canceled)

31. A method of treating dyslipidemia in a patient in need thereof, comprising administering the RNAi agent of claim 1, or a pharmaceutical composition thereof, to the patient.

32. (canceled)

33. (canceled)
